(19) United States
(12) Patent Application Publication
(10) Pub. No.: US 2020/0308171 A1
(43) Pub. Date: Oct. 1, 2020

(54) BIFUNCTIONAL INHIBITORS WITH EGFR HAVING A E3 UBIQUITIN LIGASE MOIETY

(71) Applicants: Hoffmann-La Roche Inc., Little Falls, NJ (US); C4 Therapeutics, Inc., Watertown, MA (US)

(72) Inventors: Georg Jaeschke, Basel (CH); Bernd Kuhn, Reinach BL (CH); Antonio Ricci, Biel-Benken (CH); Daniel Rueher, Raedersdorf (FR); Sandra Steiner, Sursee (CH); Yvonne Alice Nagel, Basel (CH); Martin Duplessis, Somerville, MA (US); Kiel Lazarski, Boston, MA (US); Yanke Liang, Belmont, MA (US)

(73) Assignees: Hoffmann-La Roche Inc., Little Falls, NJ (US); C4 Therapeutics, Inc., Watertown, MA (US)

(21) Appl. No.: 16/903,237

(22) Filed: Jun. 16, 2020

Related U.S. Application Data
(63) Continuation of application No. PCT/EP2018/085303, filed on Dec. 17, 2018.

(30) Foreign Application Priority Data
Dec. 18, 2017 (EP) 17208182.0

(51) Int. Cl. C07D 471/04

(52) U.S. Cl. CPC C07D 471/04

(57) ABSTRACT

Present invention provides bifunctional compounds that comprise an E3 Ubiquitin Ligase moiety that is linked to a moiety that inhibit EGFR, where the target protein can be proximate to the ubiquitin ligase to effect degradation of said protein. Present compounds are useful for the treatment of various cancers.

### CROSS-REFERENCE TO RELATED APPLICATIONS

This application is a continuation of International Application No. PCT/EP2018/085303, filed in the International Patent Cooperation Treaty, European Receiving Office on Dec. 17, 2018, which claims the benefit of European Patent Application No. 17208182.0, filed Dec. 18, 2017. The entirety of these applications are hereby incorporated by reference herein for all purposes.

### FIELD OF THE INVENTION

Present invention provides bifunctional compounds that comprise an E3 Ubiquitin Ligase moiety that is linked to a moiety that inhibit EGFR, where the target protein can be proximate to the ubiquitin ligase to effect degradation of said protein. Present compounds are useful for the treatment of various cancers.

### BACKGROUND OF THE INVENTION

The field of targeted protein degradation promoted by small molecules has been intensively studied over the last years (see for example, Collins et al., Biochem J, 2017, 474(7), 1127-47).

Protein degradation plays a role in various cellular functions, i.e. the concentrations of regulatory proteins are adjusted through degradation into small peptides to maintain health and productivity of the cells.

Cereblon is a protein that forms an E3 ubiquitin ligase complex, which ubiquinates various other proteins. Cereblon is known as primary target for anticancer thalidomide analogs. A higher expression of cereblon has been linked to the efficiency of thalidomide analogs in cancer therapy.

In the recent years, a few bifunctional compounds have been described as useful modulators of targeted ubiquitination, e.g. WO2013020557, WO2013063560, WO 2013106643, WO2015160845, WO2016011906, WO2016105518, WO2017007612, WO2017024318 and WO2017117473.

EGFR inhibitors, in particular selective inhibitors of T790M containing EGFR mutants have been described for instance in WO2014081718, WO2014210354 and Zhou et al. “Novel mutant-selective EGFR kinase inhibitors against EGFR T790M”, NATURE, (20091224), vol. 462, no. 7276, doi:10.1038/nature08622, ISSN 0028-0836, pages 1070-1074.

Bifunctional molecules for degradation of EGFR are described for instance in WO2017185036.

However, there is still an ongoing need for effective treatment of cancers.

### SUMMARY OF THE INVENTION

Present invention provides bifunctional compounds that comprise an E3 Ubiquitin Ligase moiety that is linked to a moiety that inhibit EGFR, where the target protein can be proximate to the ubiquitin ligase to effect degradation of said protein. Present compounds are useful for the treatment of various cancers. Present compounds bind to the ubiquitously expressed E3 ligase protein cereblon (CRBN) on one hand and alter the substrate specificity of the CRBN E3 ubiquitin ligase complex, resulting in breakdown of intrinsic downstream proteins. Present compounds are on the other hand selective inhibitors of T790M containing EGFR mutants.

Present invention provides compounds of formula I, or a pharmaceutically acceptable salt thereof,

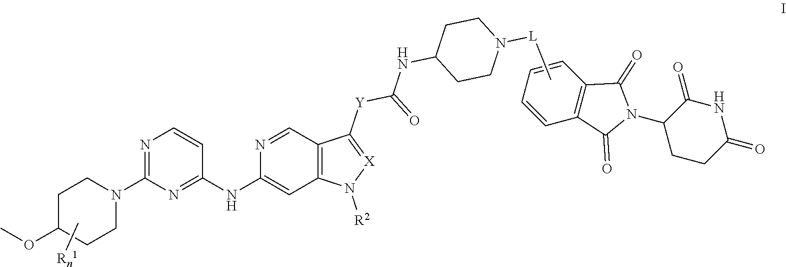

wherein the substituents and variables are as described below and in the claims, or a pharmaceutically acceptable salt thereof.

The present compounds are useful for the therapeutic and/or prophylactic treatment of cancer.

The compounds of present invention can further be used as part of bifunctional compounds that comprise the compounds of present invention as E3 Ubiquitin Ligase moiety that is linked to a moiety that binds to a target protein where the target protein is proximate to the ubiquitin ligase to effect degradation of said protein.

### DETAILED DESCRIPTION OF THE INVENTION

The present invention provides a compound of formula I and their pharmaceutically acceptable salts thereof, the preparation of the above mentioned compounds, medicaments containing them and their manufacture as well as the use of the above mentioned compounds in the therapeutic and/or prophylactic treatment of cancer.

The following definitions of the general terms used in the present description apply irrespectively of whether the terms in question appear alone or in combination with other groups.

Unless otherwise stated, the following terms used in this application, including the specification and claims, have the definitions given below. It must be noted that, as used in the specification and the appended claims, the singular forms “a”, “an,” and “the” include plural referents unless the context clearly dictates otherwise.

The term “C₁₋₆-alkyl”, alone or in combination with other groups, stands for a hydrocarbon radical which may be linear or branched, with single or multiple branching, wherein the alkyl group in general comprises 1 to 6 carbon atoms, for example, methyl (Me), ethyl (Et), propyl, isopropyl (i-propyl), n-butyl, i-butyl (isobutyl), 2-butyl (sec-butyl), t-butyl (tert-butyl), isopentyl, 2-ethyl-propyl (2-methyl-propyl), 1,2-dimethyl-propyl and the like. A specific group is isopropyl.

The term “halogen”, alone or in combination with other groups, denotes chloro (Cl), iodo (I), fluoro (F) and bromo (Br). A specific group is F.

The term “heterocyclyl” denotes a monovalent saturated or partly unsaturated mono- or bicyclic ring system of 4 to 9 ring atoms, comprising 1, 2, or 3 ring heteroatoms selected from N, O and S, the remaining ring atoms being carbon. Specific “heterocyclyl” are saturated monocyclic rings systems of 4-6 ring atoms, comprising 1-2 ring heteroatoms that are N. Examples for monocyclic saturated heterocycloalkyl are azetidinyl, pyrrolidinyl, tetrahydrofuranyl, tetrahydro-thienyl, pyrazolidinyl, imidazolidinyl, oxazolidinyl, isoxazolidinyl, thiazolidinyl, piperidyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl, or dihydropyranyl. Specific groups are azetidinyl, piperazinyl, pyrrolidinyl and piperidyl.

The terminal “C” on the left-hand side of the linker is connected to the “N” of the piperidyl moiety of the compound of formula I. A “piperidyl” or “azetidinyl” being part of a linker is linked via the “N” of the to the iso indolinyl moiety of the compound of formula I.

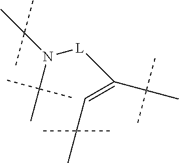

A “piperazinyl” being part of a linker is connected at both ends via the respective “N”.

If Y is pyrrolidinyl, then the “N” of the pyrrolidinyl is linked to the carbon of the bicyclic ring moiety.

The term “cycloalkyl” denotes a monovalent saturated monocyclic or bicyclic hydrocarbon group of 3 to 10 ring carbon atoms, particularly a monovalent saturated monocyclic hydrocarbon group of 3 to 8 ring carbon atoms. Particular cycloalkyl are “C₃₋₆cycloalkyl”. Bicyclic means consisting of two saturated carbocycles having one or more carbon atoms in common. Particular cycloalkyl groups are monocyclic. Examples for monocyclic cycloalkyl are cyclopropyl, cyclobutanyl, cyclopentyl, cyclohexyl or cycloheptyl, a specific example is cyclohexyl. Examples for bicyclic cycloalkyl are bicyclo[2.2.1]heptanyl, or bicyclo[2.2.2]octanyl.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

The term “a pharmaceutically acceptable salt” refers to a salt that is suitable for use in contact with the tissues of humans and animals. Examples of suitable salts with inorganic and organic acids are, but are not limited to acetic acid, citric acid, formic acid, fumaric acid, hydrochloric acid, lactic acid, maleic acid, malic acid, methane-sulfonic acid, nitric acid, phosphoric acid, p-toluenesulphonic acid, succinic acid, sulfuric acid (sulphuric acid), tartaric acid, trifluoroacetic acid and the like. Particular acids are formic acid, trifluoroacetic acid and hydrochloric acid. A specific acid is trifluoroacetic acid.

The terms “pharmaceutically acceptable auxiliary substance” refer to carriers and auxiliary substances such as diluents or excipients that are compatible with the other ingredients of the formulation.

The term “pharmaceutical composition” encompasses a product comprising specified ingredients in pre-determined amounts or proportions, as well as any product that results, directly or indirectly, from combining specified ingredients in specified amounts. Particularly it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The term “as defined herein” and “as described herein” when referring to a variable incorporates by reference the broad definition of the variable as well as particularly, more particularly and most particularly definitions, if any.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “pharmaceutically acceptable excipient” denotes any ingredient having no therapeutic activity and being non-toxic such as disintegrators, binders, fillers, solvents, buffers, tonicity agents, stabilizers, antioxidants, surfactants or lubricants used in formulating pharmaceutical products.

Whenever a chiral carbon is present in a chemical structure, it is intended that all stereoisomers associated with that chiral carbon are encompassed by the structure as pure stereoisomers as well as mixtures thereof.

The invention also provides pharmaceutical compositions, methods of using, and methods of preparing the aforementioned compounds.

All separate embodiments may be combined.
E1: One embodiment of the invention relates to a compound of formula I, or a pharmaceutically acceptable salt thereof,

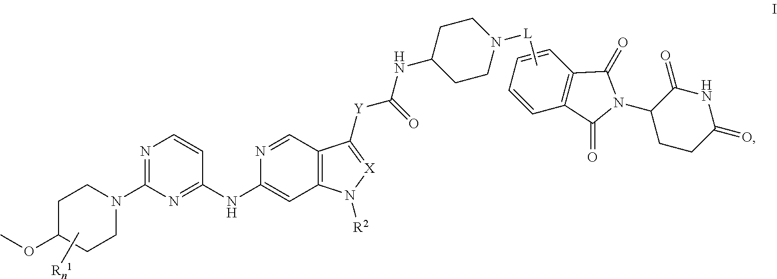

wherein
L is selected from the group consisting of
   i) —C(═O)—(CH₂)₂₋₁₀—NH—, in particular —C(═O)—(CH₂)₅—NH—;
   ii) —(CH₂)₂₋₁₀—NH—, in particular —(CH₂)₆—NH—, —(CH₂)₅—NH— or —(CH₂)₄—NH—;
   iii) —(CH₂)₂₋₁₀-heterocyclyl-, in particular
      a. —(CH₂)₂₋₁₀-piperidyl-, in particular —(CH₂)₄-piperidyl-, —(CH₂)₃-piperidyl- or —(CH₂)₂-piperidyl-; or
      b. —(CH₂)₂₋₁₀-azetidinyl-, in particular —(CH₂)₃-azetidinyl- or —(CH₂)₂-azetidinyl-;
   iv) —C(═O)-heterocyclyl-(CH₂)₂₋₁₀—NH—, in particular —C(═O)-piperazinyl-(CH₂)₂₋₁₀—NH—, more particular —C(═O)-piperazinyl-(CH₂)₄—NH—;
   v) —(CH₂)₂₋₁₀—O-heterocyclyl-, in particular
      a. —(CH₂)₂₋₁₀—O-piperidyl-, in particular —(CH₂)₃—O-piperidyl-; or
      b. —(CH₂)₂₋₁₀—O-azetidinyl-, in particular —(CH₂)₃—O-azetidinyl-; and
   vi) —(CH₂)₂₋₁₀—C₃₋₆cycloalkyl-, in particular —(CH₂)₃-cyclohexyl;
X is N or CH,
Y is absent or heterocyclyl, in particular pyrrolidinyl,
R¹ is each individually halogen, in particular F,
R² is selected from the group consisting of
   i.) C₁₋₆alkyl, in particular isopropyl, and
   ii.) C₃₋₆cycloalkyl, in particular cyclopentyl.
E2: A certain embodiment of the invention refers to the compound of formula I, or pharmaceutically acceptable salts thereof, as described herein, wherein L is selected from the group consisting of
   i) —C(═O)—(CH₂)₂₋₁₀—NH—,
   ii) —(CH₂)₂₋₁₀—NH—,
   iii) —(CH₂)₂₋₁₀-heterocyclyl-,
   iv) —C(═O)-heterocyclyl-(CH₂)₂₋₁₀—NH—,
   v) —(CH₂)₂₋₁₀—O-heterocyclyl-, and
   vi) —(CH₂)₂₋₁₀—C₃₋₆cycloalkyl-,
E3: A certain embodiment of the invention refers to the compound of formula I, or pharmaceutically acceptable salts thereof, as described herein, wherein L is selected from the group consisting of
   i) —C(═O)—(CH₂)₅—NH—,
   ii) —(CH₂)₆—NH—,
   iii) —(CH₂)₅—NH—,
   iv) —(CH₂)₄—NH—,
   v) —(CH₂)₄-piperidyl-,
   vi) —(CH₂)₃-piperidyl-,
   vii) —(CH₂)₂-piperidyl-,
   viii) —(CH₂)₃-azetidinyl-,
   ix) —(CH₂)₂-azetidinyl-,
   x) —C(═O)-piperazinyl-(CH₂)₄—NH—,
   xi) —(CH₂)₃—O-piperidyl-,
   xii) —(CH₂)₃—O-azetidinyl-, and
   xiii) —(CH₂)₃-cyclohexyl.
E4: A certain embodiment of the invention refers to the compound of formula I, or pharmaceutically acceptable salts thereof, as described herein, wherein L is —(CH₂)₂₋₁₀—NH—, in particular —(CH₂)₆—NH—, —(CH₂)₅—NH— or —(CH₂)₄—NH—.
E5: A certain embodiment of the invention refers to the compound of formula I, or pharmaceutically acceptable salts thereof, as described herein, wherein X is CH.
E6: A certain embodiment of the invention refers to the compound of formula I, or pharmaceutically acceptable salts thereof, as described herein, wherein Y is absent.
E7: A certain embodiment of the invention refers to the compound of formula I, or pharmaceutically acceptable salts thereof, as described herein, wherein R² is C₁₋₆alkyl, in particular isopropyl.
E8: A certain embodiment of the invention refers to the compound of formula I, or pharmaceutically acceptable salts thereof, as described herein, wherein R² is C₁₋₆alkyl.
E9: A certain embodiment of the invention refers to the compound of formula I, or pharmaceutically acceptable salts thereof, as described herein, wherein R² is isopropyl.
E10: A certain embodiment of the invention refers to the compound of formula I, or pharmaceutically acceptable salts thereof, as described herein, wherein R² is C₃₋₇cycloalkyl, in particular cyclopentyl.
E11: A certain embodiment of the invention refers to the compound of formula I, or pharmaceutically acceptable salts thereof, as described herein, wherein R² is C₃₋₇cycloalkyl.
E12: A certain embodiment of the invention refers to the compound of formula I, or pharmaceutically acceptable salts thereof, as described herein, wherein R² is cyclopentyl.
E13: A certain embodiment of the invention refers to the compound of formula I, or pharmaceutically acceptable salts thereof, as described herein, selected from the group consisting of
(3RS)—N-[1-[6-[[2-[(3RS)-2,6-Dioxo-3-piperidyl]-1,3-dioxo-isoindolin-4-yl]amino]hexyl]-4-piperidyl]-1-[1-isopropyl-6-[[2-(4-methoxy-1-piperidyl)pyrimidin-4-yl]amino]pyrazolo[4,3-c]pyridin-3-yl]pyrrolidine-3-carboxamide,
1-Cyclopentyl-N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3R,4 S)-3-fluoro-4-methoxy-1-piperidyl]pyrimidin-4-yl]amino]pyrrolo[3,2-c]pyridine-3-carboxamide,
1-cyclopentyl-N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide,
1-Cyclopentyl-N-[1-[3-[[1-[2-[(3 S)-2,6-dioxo-3-piperidyl]-1,3-dioxo-isoindolin-4-yl]-4-piperidyl]oxy]propyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide,
1-cyclopentyl-N-[1-[3-[[1-[2-[(3 S)-2,6-dioxo-3-piperidyl]-1,3-dioxo-isoindolin-4-yl]-4-piperidyl]oxy]propyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide,
1-Cyclopentyl-N-[1-[4-[4-[[2-[(3RS)-2,6-dioxo-3-piperidyl]-1,3-dioxo-isoindolin-4-yl]amino]butyl]piperazine-1-carbonyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide,
1-cyclopentyl-N-[1-[4-[4-[[2-[(3RS)-2,6-dioxo-3-piperidyl]-1,3-dioxo-isoindolin-4-yl]amino]butyl]piperazine-1-carbonyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide,
1-Cyclopentyl-N-[1-[5-[[2-[(3RS)-2,6-dioxo-3-piperidyl]-1,3-dioxo-isoindolin-4-yl]amino]pentyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide,
1-cyclopentyl-N-[1-[5-[[2-[(3RS)-2,6-dioxo-3-piperidyl]-1,3-dioxo-isoindolin-4-yl]amino]pentyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide,
1-Cyclopentyl-N-[1-[6-[[2-[(3RS)-2,6-dioxo-3-piperidyl]-1,3-dioxo-isoindolin-4-yl]amino]hexyl]-4-piperidyl]-6-[[2-(4-methoxy-1-piperidyl)pyrimidin-4-yl]amino]pyrrolo[3,2-c]pyridine-3-carboxamide,
1-Cyclopentyl-N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide,
1-cyclopentyl-N-[1-[6-[[2-[(3RS)-2,6-dioxo-3-piperidyl]-1,3-dioxo-isoindolin-4-yl]amino]hexyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide,
6-[[2-[(4RS)-3,3-Difluoro-4-methoxy-1-piperidyl]pyrimidin-4-yl]amino]-N-[1-[6-[[2-[(3RS)-2,6-dioxo-3-piperidyl]-1,3-dioxo-isoindolin-4-yl]amino]hexyl]-4-piperidyl]-1-isopropyl-pyrrolo[3,2-c]pyridine-3-carboxamide,
N-[1-[2-[1-[2-[(3RS)-2,6-Dioxo-3-piperidyl]-1,3-dioxo-isoindolin-5-yl]-4-piperidyl]ethyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide,
N-[1-[2-[1-[2-[(3RS)-2,6-Dioxo-3-piperidyl]-1,3-dioxo-isoindolin-4-yl]azetidin-3-yl]ethyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[3-[[1-[2-[(3RS)-2,6-Dioxo-3-piperidyl]-1,3-dioxo-isoindolin-4-yl]-4-piperidyl]oxy]propyl]-4-piperidyl]-6-[[2-[(3R,4 S)-3-fluoro-4-methoxy-1-piperidyl]pyrimidin-4-yl]amino]-1-isopropyl-pyrrolo[3,2-c]pyridine-3-carboxamide,
N-[1-[3-[[1-[2-[(3RS)-2,6-dioxo-3-piperidyl]-1,3-dioxo-isoindolin-4-yl]-4-piperidyl]oxy]propyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide,
N-[1-[3-[1-[2-[(3RS)-2,6-Dioxo-3-piperidyl]-1,3-dioxo-isoindolin-4-yl]-4-piperidyl]propyl]-4-piperidyl]-6-[[2-[(3R,4S)(3 S,4R)-3-fluoro-4-methoxy-1-piperidyl]pyrimidin-4-yl]amino]-1-isopropyl-pyrrolo[3,2-c]pyridine-3-carboxamide trifluoroacetate,
N-[1-[3-[1-[2-[(3RS)-2,6-Dioxo-3-piperidyl]-1,3-dioxo-isoindolin-4-yl]azetidin-3-yl]oxypropyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[3-[1-[2-[(3RS)-2,6-dioxo-3-piperidyl]-1,3-dioxo-isoindolin-4-yl]azetidin-3-yl]oxypropyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide,
N-[1-[3-[1-[2-[(3RS)-2,6-Dioxo-3-piperidyl]-1,3-dioxo-isoindolin-4-yl]azetidin-3-yl]propyl]-4-piperidyl]-6-[[2-[(3R,4 S)-3-fluoro-4-methoxy-1-piperidyl]pyrimidin-4-yl]amino]-1-isopropyl-pyrrolo[3,2-c]pyridine-3-carboxamide,
N-[1-[3-[4-[[2-[(3RS)-2,6-Dioxo-3-piperidyl]-1,3-dioxo-isoindolin-4-yl]amino]cyclohexyl]propyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[3-[4-[[2-[(3RS)-2,6-dioxo-3-piperidyl]-1,3-dioxo-isoindolin-4-yl]amino]cyclohexyl]propyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide,
N-[1-[4-[[2-[(3RS)-2,6-Dioxo-3-piperidyl]-1,3-dioxo-isoindolin-4-yl]amino]butyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[4-[[2-[(3RS)-2,6-dioxo-3-piperidyl]-1,3-dioxo-isoindolin-4-yl]amino]butyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide,
N-[1-[4-[1-[2-[(3RS)-2,6-Dioxo-3-piperidyl]-1,3-dioxo-isoindolin-5-yl]-4-piperidyl]butyl]-4-piperidyl]-6-[[2-[(3R,4S)(3 S,4R)-3-fluoro-4-methoxy-1-piperidyl]pyrimidin-4-yl]amino]-1-isopropyl-pyrrolo[3,2-c]pyridine-3-carboxamide,
N-[1-[5-[[2-[(3RS)-2,6-Dioxo-3-piperidyl]-1,3-dioxo-isoindolin-4-yl]amino]pentyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[5-[[2-[(3RS)-2,6-dioxo-3-piperidyl]-1,3-dioxo-isoindolin-4-yl]amino]pentyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexanoyl]-4-piperidyl]-1-isopropyl-6-[[2-(4-methoxy-1-piperidyl)pyrimidin-4-yl]amino]pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexyl]-4-piperidyl]-1-isopropyl-6-[[2-(4-methoxy-1-piperidyl)pyrimidin-4-yl]amino]pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexyl]-4-piperidyl]-6-[[2-[(3R,4S)(3 S,4R)-3-fluoro-4-methoxy-1-piperidyl]pyrimidin-4-yl]amino]-1-isopropyl-pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide,
N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[6-[[2-[(3RS)-2,6-dioxo-3-piperidyl]-1,3-dioxo-isoindolin-4-yl]amino]hexyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexyl]-4-piperidyl]-6-[[2-[(3 S,4R)-3-fluoro-4-methoxy-1-piperidyl]pyrimidin-4-yl]amino]-1-isopropyl-pyrazolo[4,3-c]pyridine-3-carboxamide, and
N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrazolo[4,3-c]pyridine-3-carboxamide.
   E14: A certain embodiment of the invention refers to the compound of formula I, or pharmaceutically acceptable salts thereof, as described herein, for use as medicament.
   E15: A certain embodiment of the invention relates to the compound of formula I as described herein, or a pharmaceutically acceptable salt thereof, for use as therapeutically active substance.
   E16: A certain embodiment of the invention relates to the compound of formula I as described herein, or a pharmaceutically acceptable salt thereof, for the use in the therapeutic and/or prophylactic treatment of cancer.
   E17: A certain embodiment of the invention relates to the compound of formula I as described herein, or a pharmaceutically acceptable salt thereof, for the manufacture of a medicament for the therapeutic and/or prophylactic treatment of cancer.
   E18: A certain embodiment of the invention relates to a pharmaceutical composition comprising the compound of formula I as described herein, or a pharmaceutically acceptable salt thereof, and a pharmaceutically acceptable auxiliary substance.
   E19: A certain embodiment of the invention relates to a method for the therapeutic and/or prophylactic treatment of cancer, by administering the compound of formula I as described herein, or a pharmaceutically acceptable salt thereof, to a patient.

Furthermore, the invention includes all optical isomers, i.e. diastereoisomers, diastereomeric mixtures, racemic mixtures, all their corresponding enantiomers and/or tautomers as well as their solvates of the compounds of formula I.

The compounds of formula I may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where optically pure enantiomers are provided, optically pure enantiomer means that the compound contains >90% of the desired isomer by weight, particularly >95% of the desired isomer by weight, or more particularly >99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

The compounds of formula I may be prepared in accordance with the schemes described in the examples. The starting material is commercially available or may be prepared in accordance with known methods.

The preparation of compounds of formula I is further described in more detail in the scheme below.

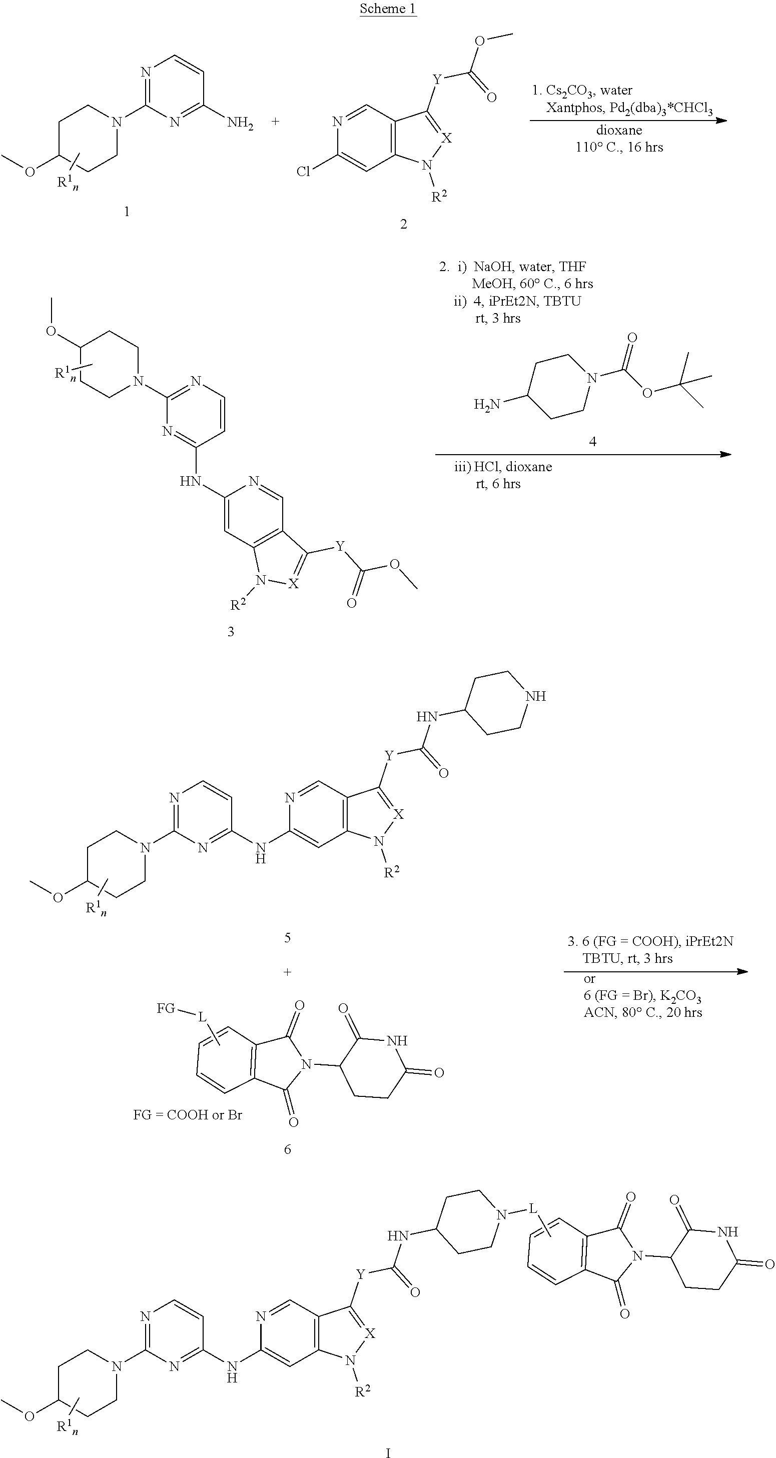

A compound of general formula I can be obtained for example by Buchwald-Hartwig cross coupling of an appropriately substituted amine 1 with a corresponding chloropyridine 2 yielding the ester derivatives of formula 3. Ester hydrolysis followed by amide coupling with a boc-protected amino derivative 4 and subsequent deprotection yields the desired piperidine 5.

Amide coupling or alkylation of 5 with an appropriate pomalidomide substituted derivative of formula 6 forms the desired final compound of general formula I (scheme 1).

Generally speaking, the sequence of steps used to synthesize the compounds of formula I can also be modified in certain cases.

Isolation and Purification of the Compounds

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used. Racemic mixtures of chiral compounds of formula I can be separated using chiral HPLC. Racemic mixtures of chiral synthetic intermediates may also be separated using chiral HPLC.

Salts of Compounds of Formula I

In cases where the compounds of formula I are basic they may be converted to a corresponding acid addition salt. The conversion is accomplished by treatment with at least a stoichiometric amount of an appropriate acid, such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Typically, the free base is dissolved in an inert organic solvent such as diethyl ether, ethyl acetate, chloroform, ethanol or methanol and the like, and the acid added in a similar solvent. The temperature is maintained between 0° C. and 50° C. The resulting salt precipitates spontaneously or may be brought out of solution with a less polar solvent.

Insofar as their preparation is not described in the examples, the compounds of formula I as well as all intermediate products can be prepared according to analogous methods or according to the methods set forth herein. Starting materials are commercially available, known in the art or can be prepared by methods known in the art or in analogy thereto.

It will be appreciated that the compounds of general formula I in this invention may be derivatised at functional groups to provide derivatives which are capable of conversion back to the parent compound in vivo.

### Pharmacological Tests

The compounds of formula I and their pharmaceutically acceptable salts possess valuable pharmacological properties. The compounds were investigated in accordance with the test given hereinafter.

EGFR Degradation Assay (Cellular)

### Generation of BaF3 EGFR Mutant Cell Lines

The BaF3 parental line was purchased from DSMZ and grown in RPMI media supplemented with 10% FBS and 10 ng/mL interleukin 3 (IL-3) (Thermo Fisher Scientific). EGFR mutants (T790M/L853R, T790M/L853R/C797S) were cloned into the pCDH lentiviral vector (SystemBio) under the control of a PGK promoter and confirmed by DNA sequencing. The resulting gene expression vector for each mutant was mixed with packaging vectors and cotransfected into 2×10E6 HEK293T cells (ATCC) in 10 mL of DMEM media to generate lentiviral particles according to the manufacturers protocol (Origene).

Three days post-transfection, the viral supernatant was harvested and filtered. In one well of a 12-well plate, 0.5 mL of viral supernatant was added to 2E6 Ba/F3 cells contained in 1.5 mL of RPMI media including 10% FBS, 10 ng/mL IL-3, and 5 μg/mL polybrene (Invitrogen). The plate was centrifuged at 2,000 rpm for 1 hour at room temperature and infected cells were kept in a tissue culture incubator overnight at 37° C. The cells were washed once in fresh BaF3 media and reseeded at 0.5E6 cells/well of a 12-well plate in media supplemented with 0.5 μg/mL puromycin. The cells were maintained in this media for 3 weeks. IL-3-independent, EGFR mutant transformed cells were routinely maintained in RPMI medium supplemented with 10% FBS.

### Materials

RPMI 1640 no-phenol red medium and fetal bovine serum (FBS) were purchased from Gibco (Grand Island, N.Y., USA). EGFR total kit and EGFR phospho-Y1068 kit were purchased from Cisbio (Bedford, Mass., USA). BaF3 EGFR mutant cell lines (EGFR T790M/L858R/C797S) cell line was generated in house, according to the protocol reported above. Cell culture flasks and 384-well microplates were acquired from VWR (Radnor, Pa., USA).

### EGFR Degradation Analysis

EGFR degradation was determined based on quantification of FRET signal using EGFR total kit. The FRET signal detected correlates with total EGFR protein level in cells. Briefly, test compounds were added to the 384-well plate from a top concentration of 1 μM with 11 points, half log titration in quadruplicates. Then, BaF3 EGFR mutant cell lines (EGFR T790M/L858R/C797S) were added into 384-well plates at a cell density of 10000 cells per well. The plates were kept at 37° C. with 5% CO2 for 4 hours. After 4-hour incubation, 4× lysis buffer was added to the cells, and then microplate was agitated on plate shaker at 500 rpm for 30 minutes at room temperature. Next, total EGFR antibody solution was added to the cells and the cells were incubated for another 4 hours at room temperature. Finally, FRET signal was acquired on EnVision™ Multilabel Reader (PerkinElmer, Santa Clara, Calif., USA). The cells treated in the absence of the test compound were the negative control and lysis buffer with antibody solution only were the positive control.

TABLE 1
IC₅₀ value

| | | IC₅₀ [nM] |
|---|---|---|
| | | mutant EGFR |
| Ex. | Name | degradation |
| 1 | N-[1-[6-[[2-[(3RS)-2,6-Dioxo-3-piperidyl]-1,3-dioxo- | 59 nM |
| | isoindolin-4-yl]amino]hexanoyl]-4-piperidyl]-1- | |
| | isopropyl-6[[2-(4-methoxy-1-piperidyl)pyrimidin-4- | |
| | yl]amino]pyrrolo[3,2-c]pyridine-3-carboxamide | |
| 2 | N-[1-[6-[[2-[(3RS)-2,6-Dioxo-3-piperidyl]-1,3-dioxo- | 132 nM |
| | isoindolin-4-yl]amino]hexyl]-4-piperidyl]-1- | |
| | isopropyl-6[[2-(4-methoxy-1-piperidyl)pyrimidin-4- | |
| | yl]amino]pyrrolo[3,2-c]pyridine-3-carboxamide | |
| 3 | 6[[2-[(4RS)-3,3-Difluoro-4-methoxy-1- | 44 nM |
| | piperidyl]pyrimidin-4-yl]amino]-N-[1[6-[[2-[(3RS)- | |
| | 2,6-dioxo-3-piperidyl]-1,3-dioxo-isoindolin-4- | |
| | yl[amino]hexyl]-4-piperidyl]-1-isopropyl-pyrrolo[3,2- | |
| | c]pyridine-3-carboxamide | |
| 4 | 1-Cyclopentyl-N-[1-[6-[[2-[(3RS)-2,6-dioxo-3- | 38 nM |
| | piperidyl]-1,3-dioxo-isoindolin-4-yl]amino]hexyl]-4- | |
| | piperidyl]-6[[2-(4-methoxy-1-piperidyl)pyrimidin-4- | |
| | yl]amino]pyrrolo[3,2-c]pyridine-3-carboxamide | |
| 5 | N-[1-[6-[[2-[(3RS)-2,6-Dioxo-3-piperidyl]-1,3-dioxo- | 28 nM |
| | isoindolin-4-yl]amino]hexyl]-4-piperidyl]-6[[2- | |
| | [(3R,4S)(3S,4R)-3-fluoro-4-methoxy-1- | |
| | piperidyl]pyrimidin-4-yl]amino]-1-isopropyl- | |
| | pyrrolo[3,2-c]pyridine-3-carboxamide | |
| 6 | (3RS)-N[1-[6-[[2-[(3RS)-2,6-Dioxo-3-piperidyl]-1,3- | 46 nM |
| | dioxo-isoindolin-4-yl]amino]hexyl]-4-piperidyl]-1[1- | |
| | isopropyl-6-[[2-(4-methoxy-1-piperidyl)pyrimidin-4- | |
| | yl]amino]pyrazolo[4,3-c]pyridin-3-yl]pyrrolidine-3- | |
| | carboxamide | |
| 7 | N-[1-[6-[[2-[(3RS)-2,6-Dioxo-3-piperidyl]-1,3-dioxo- | 70 nM |
| | isoindolin-4-yl]amino]hexyl]-4-piperidyl]-6[[2- | |
| | [(3S,4R)-3-fluoro-4-methoxy-1-piperidyl]pyrimidin- | |
| | 4-yl]amino]-1-isopropyl-pyrrolo[3,2-c]pyridine-3- | |
| | carboxamide | |
| | or | |
| | N-[1-[6-[[2-[(3RS)-2,6-dioxo-3-piperidyl]-1,3-dioxo- | |
| | isoindolin-4-yl]amino]hexyl]-4-piperidyl]-6-[2- | |
| | [(3R,4S)-3-fluoro-4-methoxy-1-piperidyl]pyrimidin- | |
| | 4-yl]amino]-1-isopropyl-pyrrolo[3,2-c]pyridine-3- | |
| | carboxamide | |
| 8 | N-[1-[6-[[2-[(3RS)-2,6-Dioxo-3-piperidyl]-1,3-dioxo- | 46 nM |
| | isoindolin-4-yl]amino]hexyl]-4-piperidyl]-6-[[2- | |
| | [(3R,4S)-3-fluoro-4-methoxy-1-piperidyl]pyrimidin- | |
| | 4-yl]amino]-1-isopropyl-pyrrolo[3,2-c]pyridine-3- | |
| | carboxamide | |
| | or | |
| | N-[1-[6-[[2-[(3RS)-2,6-dioxo-3-piperidyl]-1,3-dioxo- | |
| | isoindolin-4-yl]amino]hexyl]-4-piperidyl]-6-[[2- | |
| | [(3S,4R)-3-fluoro-4-methoxy-1-piperidyl]pyrimidin- | |
| | 4-yl]amino]-1-isopropyl-pyrrolo[3,2-c]pyridine-3- | |
| | carboxamide | |
| 9 | N-[1-[5-[[2-[(3RS)-2,6-Dioxo-3-piperidyl]-1,3-dioxo- | 26 nM |
| | isoindolin-4-yl]amino]pentyl]-4-piperidyl]-6-[[2- | |
| | [(3R,4S)-3-fluoro-4-methoxy-l-piperidyl]pyrimidin- | |
| | 4-yl]amino]-1-isopropyl-pyrrolo[3,2-c]pyridine-3- | |
| | carboxamide | |
| | or | |
| | N-[1-[5-[[2-[(3RS)-2,6-dioxo-3-piperidyl]-1,3-dioxo- | |
| | isoindolin-4-yl]amino]pentyl]-4-piperidyl]-6-[[2- | |
| | [(3S,4R)-3-fluoro-4-methoxy-1-piperidyl]pyrimidin- | |
| | 4-yl]amino]-1-isopropyl-pyrrolo[3,2-c]pyridine-3- | |
| | carboxamide | |
| 10 | 1-Cyclopentyl-N-[1-[6-[[2-[(3RS)-2,6-dioxo-3- | 85 nM |
| | piperidyl]-1,3-dioxo-isoindolin-4-yl]amino]hexyl]-4- | |
| | piperidyl]-6-[[2-[(3R,4S)-3-fluoro-4-methoxy-1- | |
| | piperidyl]pyrimidin-4-yl]amino]pyrrolo[3,2- | |
| | c]pyridine-3-carboxamide | |
| | or | |
| | 1-cyclopentyl-N-[1-[6-[[2-[(3RS)-2,6-dioxo-3- | |
| | piperidyl]-1,3-dioxo-isoindolin-4-yl]amino]hexyl]-4- | |
| | piperidyl]-6-[[2-[(3S,4R)-3-fluoro-4-methoxy-1- | |
| | piperidyl]pyrimidin-4-yl]amino]pyrrolo[3,2- | |
| | c]pyridine-3-carboxamide | |
| 11 | 1-Cyclopentyl-N-[1-[5-[[2-[(3RS)-2,6-dioxo-3- | 43 nM |
| | piperidyl]-1,3-dioxo-isoindolin-4-yl]amino]pentyl]-4- | |
| | piperidyl]-6-[[2-[(3R,4S)-3-fluoro-4-methoxy-1- | |
| | piperidyl]pyrimidin-4-yl]amino]pyrrolo[3,2- | |
| | c]pyridine-3-carboxamide | |
| | or | |
| | 1-cyclopentyl-N-[1-[5-[[2-[(3RS)-2,6-dioxo-3- | |
| | piperidyl]-1,3-dioxo-isoindolin-4-yl]amino]pentyl]-4- | |
| | piperidyl]-6-[[2-[(3S,4R)-3-fluoro-4-methoxy-1- | |
| | piperidyl]pyrimidin-4-yl]amino]pyrrolo[3,2- | |
| | c]pyridine-3-carboxamide | |
| 12 | N-[1-[3-[1-[2-[(3RS)-2,6-Dioxo-3-piperidyl]-1,3- | 69 nM |
| | dioxo-isoindolin-4-yl]-4-piperidyl]propyl]-4- | |
| | piperidyl]-6-[[2-[(3R,4S)(3S,4R)-3-fluoro-4-methoxy- | |
| | 1-piperidyl]pyrimidin-4-yl]amino]-1-isopropyl- | |
| | pyrrolo[3,2-c]pyridine-3-carboxamide trifluoroacetate | |
| 13 | 1-Cyclopentyl-N-[1-[4-[4-[[2-[(3RS)-2,6-dioxo-3- | 30 nM |
| | piperidyl]-1,3-dioxo-isoindolin-4- | |
| | yl]amino]butyl]piperazine-1-carbonyl]-4-piperidyl]-6- | |
| | [[2-[(3R,4S)-3-fluoro-4-methoxy-1- | |
| | piperidyl]pyrimidin-4-yl]amino]pyrrolo[3,2- | |
| | c]pyridine-3-carboxamide | |
| | or | |
| | 1-cyclopentyl-N-[1-[4-[4-[[2-[(3RS)-2,6-dioxo-3- | |
| | piperidyl]-1,3-dioxo-isoindolin-4- | |
| | yl]amino]butyl]piperazine-1-carbonyl]-4-piperidyl]-6- | |
| | [[2-[(3S,4R)-3-fluoro-4-methoxy-1- | |
| | piperidyl]pyrimidin-4-yl]amino]pyrrolo[3,2- | |
| | c]pyridine-3-carboxamide | |
| 14 | N-[1-[4-[[2-[(3RS)-2,6-Dioxo-3-piperidyl]-1,3-dioxo- | 41 nM |
| | isoindolin-4-yl]amino]butyl]-4-piperidyl]-6-[2- | |
| | [(3R,4S)-3-fluoro-4-methoxy-l-piperidyl]pyrimidin- | |
| | 4-yl]amino]-1-isopropyl-pyrrolo[3,2-c]pyridine-3- | |
| | carboxamide | |
| | or | |
| | N-[1-[4-[[2-[(3RS)-2,6-dioxo-3-piperidyl]-1,3-dioxo- | |
| | isoindolin-4-yl]amino]butyl]-4-piperidyl]-6-[2- | |
| | [(3S,4R)-3-fluoro-4-methoxy-1-piperidyl]pyrimidin- | |
| | 4-yl]amino]-1-isopropyl-pyrrolo[3,2-c]pyridine-3- | |
| | carboxamide | |
| 15 | 1-Cyclopentyl-N-[1-[2-[1-[2-[(3RS)-2,6-dioxo-3- | 49 nM |
| | piperidyl]-1,3-dioxo-isoindolin-5-yl]-4- | |
| | piperidyl]ethyl]-4-piperidyl]-6-[[2-[(3R,4S)-3-fluoro- | |
| | 4-methoxy-1-piperidyl]pyrimidin-4- | |
| | yl]amino]pyrrolo[3,2-c]pyridine-3-carboxamide | |
| | or | |
| | 1-cyclopentyl-N-[1-[2-[1-[2-[(3RS)-2,6-dioxo-3- | |
| | piperidyl]-1,3-dioxo-isoindolin-5-yl]-4- | |
| | piperidyl]ethyl]-4-piperidyl]-6-[[2-[(3S,4R)-3-fluoro- | |
| | 4-methoxy-1-piperidyl]pyrimidin-4- | |
| | yl]amino]pyrrolo[3,2-c]pyridine-3-carboxamide | |
| 16 | N-[1-[2-[1-[2-[(3RS)-2,6-Dioxo-3-piperidyl]-1,3- | 28 nM |
| | dioxo-isoindolin-5-yl]-4-piperidyl]ethyl]-4-piperidyl]- | |
| | 6-[[2-[(3R,4S)-3-fluoro-4-methoxy-1- | |
| | piperidyl]pyrimidin-4-yl]amino]-1-isopropyl- | |
| | pyrrolo[3,2-c]pyridine-3-carboxamide | |
| | or | |
| | N-[1-[2-[1-[2-[(3RS)-2,6-dioxo-3-piperidyl]-1,3- | |
| | dioxo-isoindolin-5-yl]-4-piperidyl]ethyl]-4-piperidyl]- | |
| | 6-[[2-[(3S,4R)-3-fluoro-4-methoxy-1- | |
| | piperidyl]pyrimidin-4-yl]amino]-1-isopropyl- | |
| | pyrrolo[3,2-c]pyridine-3-carboxamide | |
| 17 | N-[1-[3-[[1-[2-[(3RS)-2,6-Dioxo-3-piperidyl]-1,3- | 74 nM |
| | dioxo-isoindolin-4-yl]-4-piperidyl]oxy]propyl]-4- | |
| | piperidyl]-6-[2-[(3R,4S)-3-fluoro-4-methoxy-1- | |
| | piperidyl]pyrimidin-4-yl]amino]-1-isopropyl- | |
| | pyrrolo[3,2-c]pyridine-3-carboxamide | |
| | or | |
| | N-[1-[3-[[1-[2-[(3RS)-2,6-dioxo-3-piperidyl]-1,3- | |
| | dioxo-isoindolin-4-yl]-4-piperidyl]oxy]propyl]-4- | |
| | piperidyl]-6-[2-[(3S,4R)-3-fluoro-4-methoxy-1- | |
| | piperidyl]pyrimidin-4-yl]amino]-1-isopropyl- | |
| | pyrrolo[3,2-c]pyridine-3-carboxamide | |
| 18 | 1-Cyclopentyl-N-[1-[3-[[1-[2-[(3S)-2,6-dioxo-3- | 45 nM |
| | piperidyl]-1,3-dioxo-isoindolin-4-yl]-4- | |
| | piperidyl]oxy]propyl]-4-piperidyl]-6-[[2-[(3R,4S)-3- | |
| | fluoro-4-methoxy-1-piperidyl]pyrimidin-4- | |
| | yl]amino]pyrrolo[3,2-c]pyridine-3-carboxamide | |
| | or | |
| | 1-cyclopentyl-N-[1-[3-[[1-[2-[(3S)-2,6-dioxo-3- | |
| | piperidyl]-1,3-dioxo-isoindolin-4-yl]-4- | |
| | piperidyl]oxy]propyl]-4-piperidyl]-6-[[2-[(3S,4R)-3- | |
| | fluoro-4-methoxy-1-piperidyl]pyrimidin-4- | |
| | yl]amino]pyrrolo[3,2-c]pyridine-3-carboxamide | |
| 19 | N-[1-[4-[1-[2-[(3RS)-2,6-Dioxo-3-piperidyl]-1,3- | 73 nM |
| | dioxo-isoindolin-5-yl]-4-piperidyl]butyl]-4-piperidyl]- | |
| | 6-[[2-[(3R,4S)(3S,4R)-3-fluoro-4-methoxy-1- | |
| | piperidyl]pyrimidin-4-yl]amino]-1-isopropyl- | |
| | pyrrolo[3,2-c]pyridine-3-carboxamide | |
| 20 | N-(1-(3-(trans-4-((2-((3RS)-2,6-Dioxopiperidin-3-yl)- | 15 nM |
| | 1,3-dioxoisoindolin-4- | |
| | yl)amino)cyclohexyl)propyl)piperidin-4-yl)-6-((2- | |
| | ((3R,4S)-3-fluoro-4-methoxypiperidin-1- | |
| | yl)pyrimidin-4-yl)amino)-1-isopropyl-1H-pyrrolo[3,2- | |
| | c]pyridine-3-carboxamide | |
| | or | |
| | N-(1-(3-(trans-4-((2-((3RS)-2,6-dioxopiperidin-3-yl)- | |
| | 1,3-dioxoisoindolin-4- | |
| | yl)amino)cyclohexyl)propyl)piperidin-4-yl)-6-((2- | |
| | ((3S,4R)-3-fluoro-4-methoxypiperidin-1- | |
| | yl)pyrimidin-4-yl)amino)-1-isopropyl-1H-pyrrolo[3,2- | |
| | c]pyridine-3-carboxamide | |
| 21 | N-[1-[6-[[2-[(3RS)-2,6-Dioxo-3-piperidyl]-1,3-dioxo- | 2 nM |
| | isoindolin-4-yl]amino]hexyl]-4-piperidyl]-6-[2- | |
| | [(3S,4R)-3-fluoro-4-methoxy-1-piperidyl]pyrimidin- | |
| | 4-yl]amino]-1-isopropyl-pyrazolo[4,3-c]pyridine-3- | |
| | carboxamide | |
| | or | |
| | N-[1-[6-[[2-[(3RS)-2,6-dioxo-3-piperidyl]-1,3-dioxo- | |
| | isoindolin-4-yl]amino]hexyl]-4-piperidyl]-6-[2- | |
| | [(3R,4S)-3-fluoro-4-methoxy-1-piperidyl]pyrimidin- | |
| | 4-yl]amino]-1-isopropyl-pyrazolo[4,3-c]pyridine-3- | |
| | carboxamide | |
| 22 | N-[1-[3-[1-[2-[(3RS)-2,6-Dioxo-3-piperidyl]-1,3- | 26 nM |
| | dioxo-isoindolin-4-yl]azetidin-3-yl]oxypropyl]-4- | |
| | piperidyl]-6-[2-[(3R,4S)-3-fluoro-4-methoxy-1- | |
| | piperidyl]pyrimidin-4-yl]amino]-1-isopropyl- | |
| | pyrrolo[3,2-c]pyridine-3-carboxamide | |
| | or | |
| | N-[1-[3-[1-[2-[(3RS)-2,6-dioxo-3-piperidyl]-1,3- | |
| | dioxo-isoindolin-4-yl]azetidin-3-yl]oxypropyl]-4- | |
| | piperidyl]-6-[2-[(3S,4R)-3-fluoro-4-methoxy-1- | |
| | piperidyl]pyrimidin-4-yl]amino]-1-isopropyl- | |
| | pyrrolo[3,2-c]pyridine-3-carboxamide | |
| 23 | N-[1-[2-[1-[2-[(3RS)-2,6-Dioxo-3-piperidyl]-1,3- | 8 nM |
| | dioxo-isoindolin-4-yl]azetidin-3-yl]ethyl]-4- | |
| | piperidyl]-6-[[2-[(3R,4S)(3S,4R)-3-fluoro-4-methoxy- | |
| | 1-piperidyl]pyrimidin-4-yl]amino]-1-isopropyl- | |
| | pyrrolo[3,2-c]pyridine-3-carboxamide | |
| 24 | N-[1-[3-[1-[2-[(3RS)-2,6-Dioxo-3-piperidyl]-1,3- | 5 nM |
| | dioxo-isoindolin-4-yl]azetidin-3-yl]propyl]-4- | |
| | piperidyl]-6-[[2-[(3R,4S)(3S,4R)-3-fluoro-4-methoxy- | |
| | 1-piperidyl]pyrimidin-4-yl]amino]-1-isopropyl- | |
| | pyrrolo[3,2-c]pyridine-3-carboxamide | |

### Pharmaceutical Compositions

The compounds of formula I and the pharmaceutically acceptable salts can be used as therapeutically active substances, e.g. in the form of pharmaceutical preparations. The pharmaceutical preparations can be administered orally, e.g. in the form of tablets, coated tablets, dragées, hard and soft gelatin capsules, solutions, emulsions or suspensions. The administration can, however, also be effected rectally, e.g. in the form of suppositories, or parenterally, e.g. in the form of injection solutions.

The compounds of formula I and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of formula I or a pharmaceutically acceptable salt thereof and a therapeutically inert carrier are also provided by the present invention, as is a process for their production, which comprises bringing one or more compounds of formula I and/or pharmaceutically acceptable salts thereof and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparations conveniently contain about 1-500 mg, particularly 1-100 mg, of a compound of formula I. Examples of compositions according to the invention are:

### Example A

Tablets of the following composition are manufactured in the usual manner:

TABLE 2
possible tablet composition

| | mg/tablet | | | |
|---|---|---|---|---|
| ingredient | 5 | 25 | 100 | 500 |
| Compound of formula I | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### Example B-1

Capsules of the following composition are manufactured:

TABLE 3
possible capsule ingredient composition

| | mg/capsule | | | |
|---|---|---|---|---|
| ingredient | 5 | 25 | 100 | 500 |
| Compound of formula I | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula I, lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

Soft Gelatin Capsules of the following composition are manufactured:

TABLE 4
possible soft gelatin capsule ingredient composition

| ingredient | mg/capsule |
|---|---|
| Compound of formula I | 5 |
| Yellow wax | 8 |
| Hydrogenated Soya bean oil | 8 |
| Partially hydrogenated plant oils | 34 |
| Soya bean oil | 110 |
| Total | 165 |

TABLE 5
possible soft gelatin capsule composition

| ingredient | mg/capsule |
|---|---|
| Gelatin | 75 |
| Glycerol 85% | 32 |
| Karion 83 | 8 |
| | (dry matter) |
| Titan dioxide | 0.4 |
| Iron oxide yellow | 1.1 |
| Total | 116.5 |

### Manufacturing Procedure

The compound of formula I is dissolved in a warm melting of the other ingredients and the mixture is filled into soft gelatin capsules of appropriate size. The filled soft gelatin capsules are treated according to the usual procedures.

### Example C

Suppositories of the following composition are manufactured:

TABLE 6
possible suppository composition

| ingredient | mg/supp. |
|---|---|
| Compound of formula I | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula I is added thereto and stirred until it has dispersed completely. The mixture is poured into suppository moulds of suitable size, left to cool; the suppositories are then removed from the moulds and packed individually in wax paper or metal foil.

### Example D

Injection solutions of the following composition are manufactured:

TABLE 7
possible injection solution composition

| ingredient | mg/injection solution. |
|---|---|
| Compound of formula I | 3 |
| Polyethylene Glycol 400 | 150 |
| acetic acid | q.s. ad pH 5.0 |
| water for injection solutions | ad 1.0 ml |

### Manufacturing Procedure

The compound of formula I is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

Sachets of the following composition are manufactured:

TABLE 8
possible sachet composition

| ingredient | mg/sachet |
|---|---|
| Compound of formula I | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula I is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### Experimental Part

The following examples are provided for illustration of the invention. They should not be considered as limiting the scope of the invention, but merely as being representative thereof.

### Example 1

### N-[1-[6-[[2-[(3RS)-2,6-Dioxo-3-piperidyl]-1,3-dioxo-isoindolin-4-yl]amino]hexanoyl]-4-piperidyl]-1-isopropyl-6-[[2-(4-methoxy-1-piperidyl)pyrimidin-4-yl]amino]pyrrolo[3,2-c]pyridine-3-carboxamide

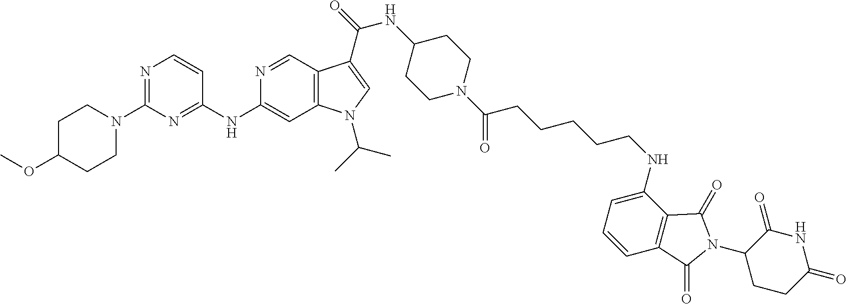 [SMILES: COC1CCN(c2nccc(Nc3cc4c(cn3)c(C(=O)NC3CCN(C(=O)CCCCCNc5cccc6c5C(=O)N(C5CCC(=O)NC5=O)C6=O)CC3)cn4C(C)C)n2)CC1]

### Step 1: 2-(4-Methoxy-1-piperidyl)pyrimidin-4-amine

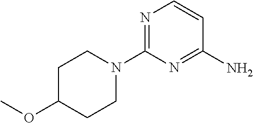

2-Chloropyrimidin-4-amine (5 g, 38.6 mmol) was dissolved in 50 ml of dioxane. 4-Methoxypiperidine (4.9 g, 42.5 mmol, 1.1 equiv.) and Hunig's base (5.5 g, 7.41 ml, 42.5 mmol, 1.1 equiv.) were added at room temperature. The mixture was stirred at 110° C. for 16 hours. The reaction mixture was evaporated to dryness and the residue suspended in diisopropylether. The solid was filtered, washed with diisopropylether and dried for 2 hours at 50° C. and <10 mbar. The desired 2-(4-methoxy-1-piperidyl)pyrimidin-4-amine (6.6 g, 82% yield) was obtained as a light yellow solid, MS: m/e=209.1 (M+H⁺).

### Step 2: Methyl 6-chloro-1-isopropyl-1H-pyrrolo[3,2-c]pyridine-3-carboxylate

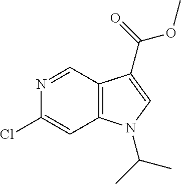 [SMILES: COC(=O)c1cn(C(C)C)c2cc(Cl)ncc12]

Methyl 6-chloro-1H-pyrrolo[3,2-c]pyridine-3-carboxylate (CAS 1784502-69-7) (5 g, 23.7 mmol) was dissolved in 50 ml of DMF and cooled to 0-5° C. Sodium hydride (60% in mineral oil) (1.14 g, 28.5 mmol, 1.2 equiv.) was added carefully in portions at 0-5° C. After 10 minutes 2-iodopropane (5.25 g, 3.1 ml, 30.9 mmol, 1.3 equiv.) was added and the mixture was stirred for 2 hours at room temperature. The reaction mixture was extracted with saturated NaHCO₃-solution and two times with TBME. The organic layers were extracted with water and brine, dried over sodium sulfate and evaporated to dryness. The crude product was purified by flash chromatography on a silica gel column eluting with an ethyl acetate:heptane 0:100 to 70:30 gradient to obtain the desired methyl 6-chloro-1-isopropyl-1H-pyrrolo[3,2-c]pyridine-3-carboxylate (3.1 g, 52% yield) as a light yellow oil, MS: m/e=252.9/254.9 (M+H⁺).

### Step 3: Methyl 1-isopropyl-6-[[2-(4-methoxy-1-piperidyl)pyrimidin-4-yl]amino]pyrrolo[3,2-c]pyridine-3-carboxylate

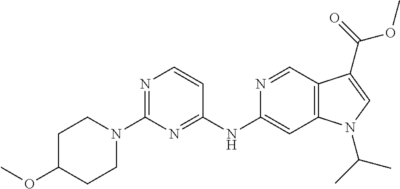

Methyl 6-chloro-1-isopropyl-1H-pyrrolo[3,2-c]pyridine-3-carboxylate (Example 1, step 2) (1.46 g, 5.76 mmol, 1.2 equiv.) was dissolved in 26 ml of dioxane. 2-(4-Methoxypiperidin-1-yl)pyrimidin-4-amine (Example 1, step 1) (1 g, 4.8 mmol), cesium carbonate (4.7 g, 14.4 mmol, 3 equiv.), water (43 mg, 0.043 ml, 2.4 mmol, 0.5 equiv.), xantphos (278 mg, 0.48 mmol, 0.1 equiv.) and tris(dibenzylideneacetone)dipalladium (0) chloroform adduct (249 mg, 0.24 mmol, 0.05 equiv.) were added under nitrogen in a sealed tube. The mixture was stirred at 110° C. for 16 hours. The reaction mixture was extracted with water and two times with ethyl acetate. The organic layers were dried over sodium sulfate and evaporated to dryness. The crude product was purified by flash chromatography on a silica gel column eluting with a dichloromethane:methanol 100:0 to 90:10 gradient to obtain the desired methyl 1-isopropyl-6-[[2-(4-methoxy-1-piperidyl)pyrimidin-4-yl]amino]pyrrolo[3,2-c]pyridine-3-carboxylate (1.44 g, 71% yield) as a light brown solid, MS: m/e=425.7 (M+H⁺).

### Step 4: 1-Isopropyl-6-[[2-(4-methoxy-1-piperidyl)pyrimidin-4-yl]amino]pyrrolo[3,2-c]pyridine-3-carboxylic acid

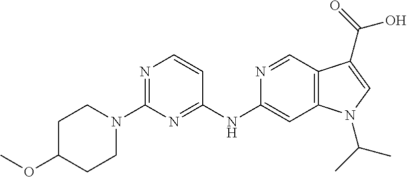 [SMILES: COC1CCN(c2nccc(Nc3cc4c(cn3)c(C(=O)O)cn4C(C)C)n2)CC1]

Methyl 1-isopropyl-6-[[2-(4-methoxy-1-piperidyl)pyrimidin-4-yl]amino]pyrrolo[3,2-c]pyridine-3-carboxylate (Example 1, step 3) (1.44 g, 3.39 mmol) was dissolved in 15 ml of THF and 4 ml of methanol. Sodium hydroxide (2N in water) (2.54 ml, 5.09 mmol, 1.5 equiv.) was added and the mixture was stirred at 60° C. for 6 hours. The reaction mixture was extracted with TBME and two times with 1N sodium hydroxide solution. The aqueous layers were combined, acidified with 1N KHSO₄ solution to pH 5. The solid was filtered, washed with water and dried to obtain the desired 1-isopropyl-6-[[2-(4-methoxy-1-piperidyl)pyrimidin-4-yl]amino]pyrrolo[3,2-c]pyridine-3-carboxylic acid (985 mg, 71% yield) as a white solid, MS: m/e=411.7 (M+H⁺).

### Step 5: tert-Butyl 4-[[1-isopropyl-6-[[2-(4-methoxy-1-piperidyl)pyrimidin-4-yl]amino]pyrrolo[3,2-c]pyridine-3-carbonyl]amino]piperidine-1-carboxylate

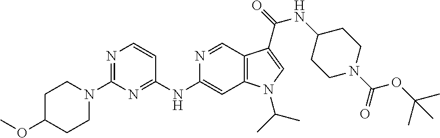 [SMILES: COC1CCN(c2nccc(Nc3cc4c(cn3)c(C(=O)NC3CCN(C(=O)OC(C)(C)C)CC3)cn4C(C)C)n2)CC1]

1-Isopropyl-6-[[2-(4-methoxy-1-piperidyl)pyrimidin-4-yl]amino]pyrrolo[3,2-c]pyridine-3-carboxylic acid (Example 1, step 4) (636 mg, 1.55 mmol) was dissolved in 15 ml of DMF. tert-Butyl 4-aminopiperidine-1-carboxylate (310 mg, 1.55 mmol, 1 equiv.), Hunig's base (1 g, 1.35 ml, 7.75 mmol, 5 equiv.) and TBTU (597 mg, 1.86 mmol, 1.2 equiv.) were added at room temperature. The mixture was stirred at room temperature for 3 hours. The reaction mixture was extracted with water and two times with ethyl acetate. The organic layers were dried over sodium sulfate and evaporated to dryness. The crude product was purified by flash chromatography on a silica gel column eluting with a dichloromethane:methanol 100:0 to 90:10 gradient to obtain the desired tert-butyl 4-[[1-isopropyl-6-[[2-(4-methoxy-1-piperidyl)pyrimidin-4-yl]amino]pyrrolo[3,2-c]pyridine-3-carbonyl]amino]piperidine-1-carboxylate (746 mg, 81% yield) as a white foam, MS: m/e=594.0 (M+H⁺).

### Step 6: 1-Isopropyl-6-[[2-(4-methoxy-1-piperidyl)pyrimidin-4-yl]amino]-N-(4-piperidyl)pyrrolo[3,2-c]pyridine-3-carboxamide hydrochloride

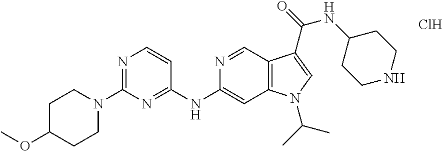 [SMILES: COC1CCN(c2nccc(Nc3cc4c(cn3)c(C(=O)NC3CCNCC3)cn4C(C)C)n2)CC1 ; Cl]

tert-Butyl 4-[[1-isopropyl-6-[[2-(4-methoxy-1-piperidyl)pyrimidin-4-yl]amino]pyrrolo[3,2-c]pyridine-3-carbonyl]amino]piperidine-1-carboxylate (Example 1, step 5) (745 mg, 1.26 mmol) was dissolved in 25 ml of methanol and HCl (4N in dioxane) (3.14 ml, 12.6 mmol, 10 equiv.) was added at room temperature. The mixture was stirred at room temperature for 6 hours. The reaction mixture was evaporated to dryness to obtain the desired 1-isopropyl-6-[[2-(4-methoxy-1-piperidyl)pyrimidin-4-yl]amino]-N-(4-piperidyl)pyrrolo[3,2-c]pyridine-3-carboxamide hydrochloride (quant. yield) as a white foam, MS: m/e=493.8 (M+H⁺).

### Step 7: 6-[[2-[(3RS)-2,6-Dioxo-3-piperidyl]-1,3-dioxo-isoindolin-4-yl]amino]hexanoic acid

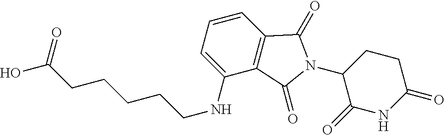 [SMILES: O=C(O)CCCCCNc1cccc2c1C(=O)N(C1CCC(=O)NC1=O)C2=O]

A mixture of 6-aminohexanoic acid (1.7 g, 13.03 mmol, 1.2 equiv.), 2-[(3RS)-2,6-dioxo-3-piperidyl]-4-fluoro-isoindoline-1,3-dione (CAS 835616-60-9) (3 g, 10.86 mmol), Hunig's base (5.7 ml, 32.58 mmol, 3 equiv.) in 50 ml of DMSO was stirred at 100° C. for 16 hours. Water (500 ml) was added to the reaction mixture and extracted four times with ethyl acetate (200.0 ml each). The combined organic layers were washed with brine, dried over sodium sulfate and concentrated to give a residue. The crude product was purified by flash chromatography on a silica gel column eluting with a petroleum ether:ethyl acetate 3:1 to 0:1 gradient and trituration in dichloromethane to obtain the desired 6-[[2-[(3RS)-2,6-dioxo-3-piperidyl]-1,3-dioxo-isoindolin-4-yl]amino]hexanoic acid (1.4 g, 31% yield) as a green solid, MS: m/e=388.1 (M+H⁺).

### Step 8: N-[1-[6-[[2-[(3RS)-2,6-Dioxo-3-piperidyl]-1,3-dioxo-isoindolin-4-yl]amino]hexanoyl]-4-piperidyl]-1-isopropyl-6-[[2-(4-methoxy-1-piperidyl)pyrimidin-4-yl]amino]pyrrolo[3,2-c]pyridine-3-carboxamide

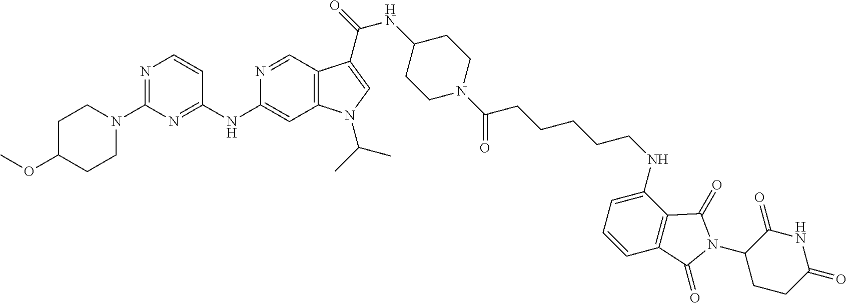 [SMILES: COC1CCN(c2nccc(Nc3cc4c(cn3)c(C(=O)NC3CCN(C(=O)CCCCCNc5cccc6c5C(=O)N(C5CCC(=O)NC5=O)C6=O)CC3)cn4C(C)C)n2)CC1]

The title compound was obtained as a yellow solid, MS: m/e=863.0 (M+H⁺), using chemistry similar to that described in Example 1, step 5 starting from (1-isopropyl-6-[[2-(4-methoxy-1-piperidyl)pyrimidin-4-yl]amino]-N-(4-piperidyl)pyrrolo[3,2-c]pyridine-3-carboxamide hydrochloride (Example 1, step 6) and 6-[[2-[(3RS)-2,6-dioxo-3-piperidyl]-1,3-dioxo-isoindolin-4-yl]amino]hexanoic acid (Example 1, step 7).

### Example 2

### N-[1-[6-[[2-[(3RS)-2,6-Dioxo-3-piperidyl]-1,3-dioxo-isoindolin-4-yl]amino]hexyl]-4-piperidyl]-1-isopropyl-6-[[2-(4-methoxy-1-piperidyl)pyrimidin-4-yl]amino]pyrrolo[3,2-c]pyridine-3-carboxamide

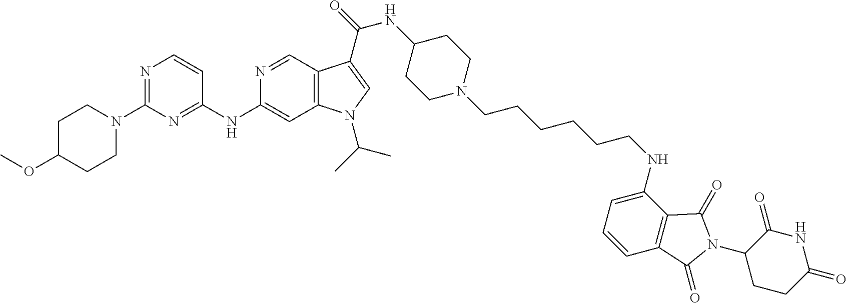 [SMILES: COC1CCN(c2nccc(Nc3cc4c(cn3)c(C(=O)NC3CCN(CCCCCCNc5cccc6c5C(=O)N(C5CCC(=O)NC5=O)C6=O)CC3)cn4C(C)C)n2)CC1]

### Step 1: 2-[(3RS)-2,6-Dioxo-3-piperidyl]-4-(6-hydroxyhexylamino)isoindoline-1,3-dione

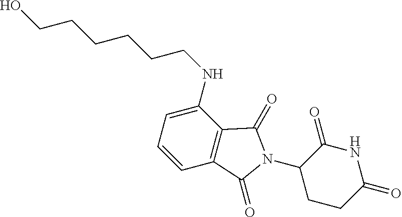 [SMILES: O=C1CCC(N2C(=O)c3cccc(NCCCCCCO)c3C2=O)C(=O)N1]

The title compound was obtained as a green oil, MS: m/e=374.2 (M+H⁺), using chemistry similar to that described in Example 1, step 7 starting from 2-[(3RS)-2,6-dioxo-3-piperidyl]-4-fluoro-isoindoline-1,3-dione (CAS 835616-60-9) and 6-amino-1-hexanol.

### Step 2: 4-(6-Bromohexylamino)-2-[(3RS)-2,6-dioxo-3-piperidyl]isoindoline-1,3-dione

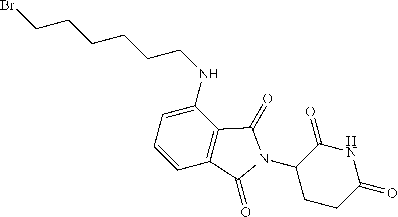 [SMILES: O=C1CCC(N2C(=O)c3cccc(NCCCCCCBr)c3C2=O)C(=O)N1]

A mixture of 2-[(3RS)-2,6-dioxo-3-piperidyl]-4-(6-hydroxyhexylamino)isoindoline-1,3-dione (Example 2, step 1) (4.0 g, 10.71 mmol), triphenylphosphine (5.62 g, 21.42 mmol, 2 equiv.) and carbon tetrabromide (7.11 g, 21.42 mmol, 2 equiv.) in THF (200 ml) was stirred at 50° C. for 3 hours. The mixture was filtered and the filtrate was concentrated to give a residue. The crude product was purified by flash chromatography on a silica gel column eluting with a petroleum ether: ethyl acetate 5:1 to 1:1 gradient to obtain the desired 4-(6-bromohexylamino)-2-[(3RS)-2,6-dioxo-3-piperidyl]isoindoline-1,3-dione (2.2 g, 45% yield) as a yellow solid, MS: m/e=436.0/438.0 (M+H⁺).

### Step 3: N-[1-[6-[[2-[(3RS)-2,6-Dioxo-3-piperidyl]-1,3-dioxo-isoindolin-4-yl]amino]hexyl]-4-piperidyl]-1-isopropyl-6-[[2-(4-methoxy-1-piperidyl)pyrimidin-4-yl]amino]pyrrolo[3,2-c]pyridine-3-carboxamide

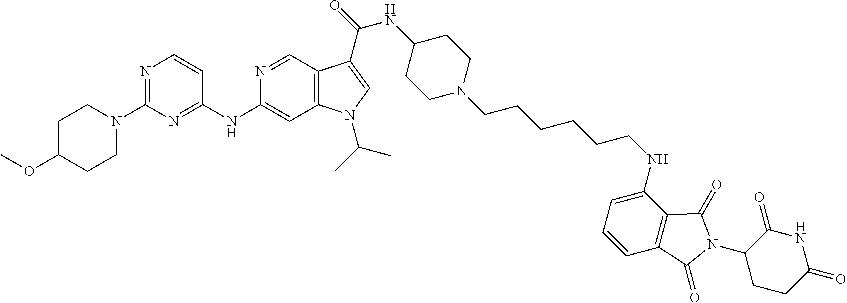 [SMILES: COC1CCN(c2nccc(Nc3cc4c(cn3)c(C(=O)NC3CCN(CCCCCCNc5cccc6c5C(=O)N(C5CCC(=O)NC5=O)C6=O)CC3)cn4C(C)C)n2)CC1]

(1-Isopropyl-6-[[2-(4-methoxy-1-piperidyl)pyrimidin-4-yl]amino]-N-(4-piperidyl)pyrrolo[3,2-c]pyridine-3-carboxamide hydrochloride (Example 1, step 6) (60 mg, 0.113 mmol) was dissolved in 6 ml of acetonitrile. 4-(6-Bromohexylamino)-2-[(3RS)-2,6-dioxo-3-piperidyl]isoindoline-1,3-dione (Example 2, step 2) (50 mg, 0.113 mmol, 1 equiv.) and potassium carbonate (125 mg, 0.91 mmol, 8 equiv.) were added at room temperature. The mixture was stirred at 80° C. for 20 hours. The reaction mixture was extracted with water and several times with dichlormethane:methanol 9:1 mixture. The organic layers were dried over sodium sulfate and evaporated to dryness. The crude product was purified by flash chromatography on a silica gel column eluting with a dichloromethane:methanol 100:0 to 75:25 gradient to obtain the desired N-[1-[6-[[2-[(3RS)-2,6-dioxo-3-piperidyl]-1,3-dioxo-isoindolin-4-yl]amino]hexyl]-4-piperidyl]-1-isopropyl-6-[[2-(4-methoxy-1-piperidyl)pyrimidin-4-yl]amino]pyrrolo[3,2-c]pyridine-3-carboxamide (48 mg, 50% yield) as a yellow semisolid, MS: m/e=849.1 (M+H⁺).

### Example 3

### 6-[[2-[(4RS)-3,3-Difluoro-4-methoxy-1-piperidyl]pyrimidin-4-yl]amino]-N-[1-[6-[[2-[(3RS)-2,6-dioxo-3-piperidyl]-1,3-dioxo-isoindolin-4-yl]amino]hexyl]-4-piperidyl]-1-isopropyl-pyrrolo[3,2-c]pyridine-3-carboxamide

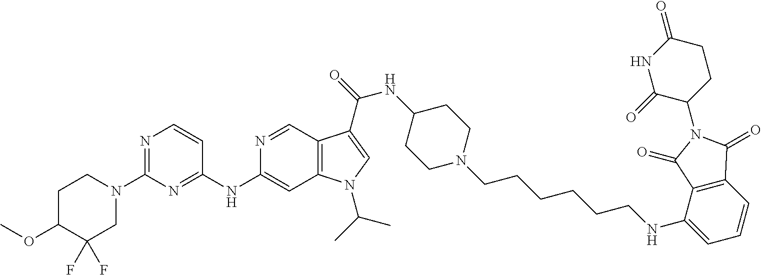 [SMILES: COC1CCN(c2nccc(Nc3cc4c(cn3)c(C(=O)NC3CCN(CCCCCCNc5cccc6c5C(=O)N(C5CCC(=O)NC5=O)C6=O)CC3)cn4C(C)C)n2)CC1(F)F]

### Step 1: 2-[(4RS)-3,3-Difluoro-4-methoxy-1-piperidyl]pyrimidin-4-amine

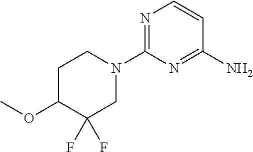 [SMILES: COC1CCN(c2nccc(N)n2)CC1(F)F]

The title compound was obtained as a brown solid, MS: m/e=245.0 (M+H⁺), using chemistry similar to that described in Example 1, step 1 starting from 2-chloropyrimidin-4-amine and (4RS)-3,3-difluoro-4-methoxy-piperidine (CAS 1373609-11-0).

### Step 2: 6-[[2-[(4RS)-3,3-Difluoro-4-methoxy-1-piperidyl]pyrimidin-4-yl]amino]-1-isopropyl-N-(4-piperidyl)pyrrolo[3,2-c]pyridine-3-carboxamide hydrochloride

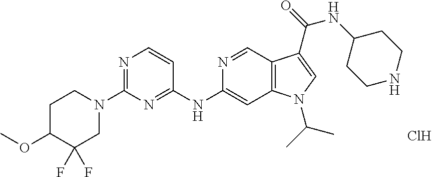 [SMILES: COC1CCN(c2nccc(Nc3cc4c(cn3)c(C(=O)NC3CCNCC3)cn4C(C)C)n2)CC1(F)F ; Cl]

The title compound was obtained as a white foam, MS: m/e=529.7 (M+H⁺), using chemistry similar to that described in Example 1, step 3 to step 6 starting from methyl 6-chloro-1-isopropyl-1H-pyrrolo[3,2-c]pyridine-3-carboxylate (Example 1, step 2) and 2-[(4RS)-3,3-difluoro-4-methoxy-1-piperidyl]pyrimidin-4-amine (Example 3, step 1).

### Step 3: 6-[[2-[(4RS)-3,3-Difluoro-4-methoxy-1-piperidyl]pyrimidin-4-yl]amino]-N-[1-[6-[[2-[(3RS)-2,6-dioxo-3-piperidyl]-1,3-dioxo-isoindolin-4-yl]amino]hexyl]-4-piperidyl]-1-isopropyl-pyrrolo[3,2-c]pyridine-3-carb oxamide

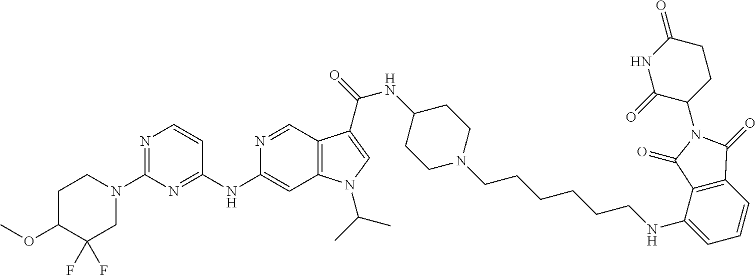 [SMILES: COC1CCN(c2nccc(Nc3cc4c(cn3)c(C(=O)NC3CCN(CCCCCCNc5cccc6c5C(=O)N(C5CCC(=O)NC5=O)C6=O)CC3)cn4C(C)C)n2)CC1(F)F]

The title compound was obtained as a yellow solid, MS: m/e=885.0 (M+H⁺), using chemistry similar to that described in Example 2, step 3 starting from 6-[[2-[(416)-3,3-difluoro-4-methoxy-1-piperidyl]pyrimidin-4-yl]amino]-1-isopropyl-N-(4-piperidyl)pyrrolo[3,2-c]pyridine-3-carboxamide hydrochloride (Example 3, step 2) and 4-(6-bromohexylamino)-2-[(3RS)-2,6-dioxo-3-piperidyl]isoindoline-1,3-dione (Example 2, step 2).

### Example 4

### 1-Cyclopentyl-N-[1-[6-[[2-[(3RS)-2,6-dioxo-3-piperidyl]-1,3-dioxo-isoindolin-4-yl]amino]hexyl]-4-piperidyl]-6-[[2-(4-methoxy-1-piperidyl)pyrimidin-4-yl]amino]pyrrolo[3,2-c]pyridine-3-carboxamide

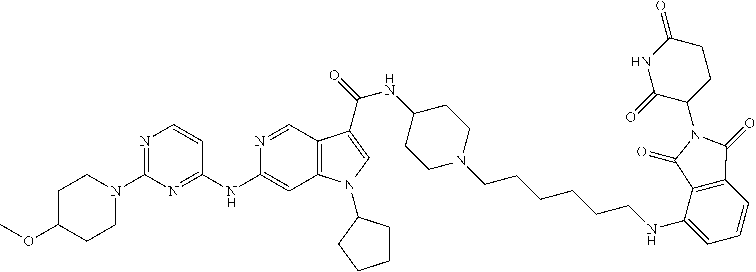 [SMILES: COC1CCN(c2nccc(Nc3cc4c(cn3)c(C(=O)NC3CCN(CCCCCCNc5cccc6c5C(=O)N(C5CCC(=O)NC5=O)C6=O)CC3)cn4C3CCCC3)n2)CC1]

### Step 1: Methyl 6-chloro-1-cyclopentyl-1H-pyrrolo[3,2-c]pyridine-3-carboxylate

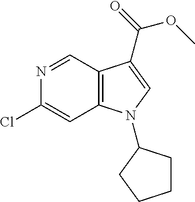 [SMILES: COC(=O)c1cn(C2CCCC2)c2cc(Cl)ncc12]

The title compound was obtained as a white solid, MS: m/e=279.4 (M+H⁺), using chemistry similar to that described in Example 1, step 2 starting from methyl 6-chloro-1H-pyrrolo[3,2-c]pyridine-3-carboxylate (CAS 1784502-69-7) and iodocyclopentane.

### Step 2: 1-Cyclopentyl-6-[[2-(4-methoxy-1-piperidyl)pyrimidin-4-yl]amino]-N-(4-piperidyl)pyrrolo[3,2-c]pyridine-3-carboxamide hydrochloride

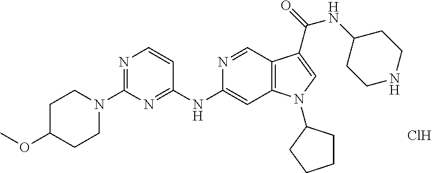 [SMILES: COC1CCN(c2nccc(Nc3cc4c(cn3)c(C(=O)NC3CCNCC3)cn4C3CCCC3)n2)CC1 ; Cl]

The title compound was obtained as a light yellow solid, MS: m/e=519.7 (M+H⁺), using chemistry similar to that described in Example 1, step 3 to step 6 starting from methyl 6-chloro-1-cyclopentyl-1H-pyrrolo[3,2-c]pyridine-3-carboxylate (Example 4, step 1) and 2-(4-methoxypiperidin-1-yl)pyrimidin-4-amine (Example 1, step 1).

### Step 3: 1-Cyclopentyl-N-[1-[6-[2-[(3RS)-2,6-dioxo-3-piperidyl]-1,3-dioxo-isoindolin-4-yl]amino]hexyl]-4-piperidyl]-6-[[2-(4-methoxy-1-piperidyl)pyrimidin-4-yl]amino]pyrrolo[3,2-c]pyridine-3-carboxamide

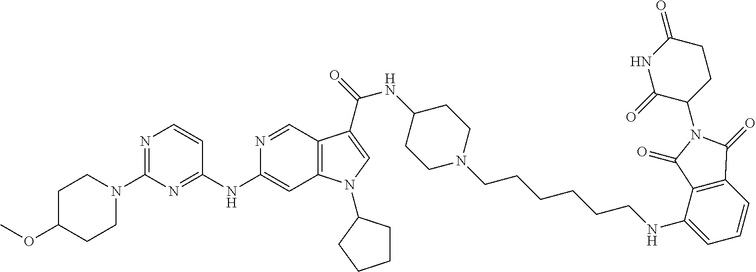 [SMILES: COC1CCN(c2nccc(Nc3cc4c(cn3)c(C(=O)NC3CCN(CCCCCCNc5cccc6c5C(=O)N(C5CCC(=O)NC5=O)C6=O)CC3)cn4C3CCCC3)n2)CC1]

The title compound was obtained as a yellow solid, MS: m/e=874.9 (M+H⁺), using chemistry similar to that described in Example 2, step 3 starting from 1-cyclopentyl-6-[[2-(4-methoxy-1-piperidyl)pyrimidin-4-yl]amino]-N-(4-piperidyl)pyrrolo[3,2-c]pyridine-3-carboxamide hydrochloride (Example 4, step 2) and 4-(6-bromohexylamino)-2-[(3RS)-2,6-dioxo-3-piperidyl]isoindoline-1,3-dione (Example 2, step 2).

### Example 5

### N-[1-[6-[[2-[(3RS)-2,6-Dioxo-3-piperidyl]-1,3-dioxo-isoindolin-4-yl]amino]hexyl]-4-piperidyl]-6-[[2-[(3R,4S)(3S,4R)-3-fluoro-4-methoxy-1-piperidyl]pyrimidin-4-yl]amino]-1-isopropyl-pyrrolo[3,2-c]pyridine-3-carboxamide

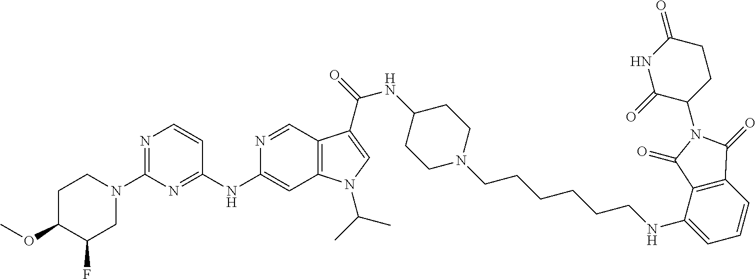 [SMILES: CO[C@H]1CCN(c2nccc(Nc3cc4c(cn3)c(C(=O)NC3CCN(CCCCCCNc5cccc6c5C(=O)N(C5CCC(=O)NC5=O)C6=O)CC3)cn4C(C)C)n2)C[C@H]1F]

### Step 1: 2-((3R,4S)(3 S,4R)-3-Fluoro-4-methoxypiperidin-1-yl)pyrimidin-4-amine

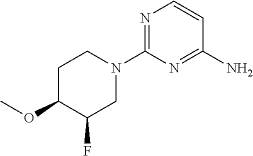 [SMILES: CO[C@H]1CCN(c2nccc(N)n2)C[C@H]1F]

The title compound was obtained as a yellow solid, MS: m/e=227.0 (M+H+), using chemistry similar to that described in Example 1, step 1 starting from 2-chloropyrimidin-4-amine and (3R,4S) (3S,4R)-3-fluoro-4-methoxy-piperidine hydrochloride (CAS 1147110-70-0).

### Step 2: 6-((2-((3R,4S)(3 S,4R)-3-Fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride

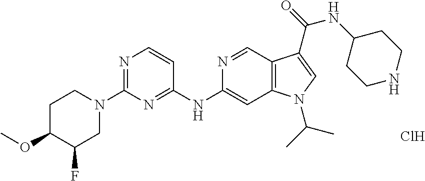 [SMILES: CO[C@H]1CCN(c2nccc(Nc3cc4c(cn3)c(C(=O)NC3CCNCC3)cn4C(C)C)n2)C[C@H]1F ; Cl]

The title compound was obtained as a white foam, MS: m/e=511.7 (M+H⁺), using chemistry similar to that described in Example 1, step 3 to step 6 starting from methyl 6-chloro-1-isopropyl-1H-pyrrolo[3,2-c]pyridine-3-carboxylate (Example 1, step 2) and 2-((3R,4 S)(3 S,4R)-3-fluoro-4-methoxypiperidin-1-yl)pyrimidin-4-amine (Example 5, step 1).

### Step 3: N-[1-[6-[[2-[(3RS)-2,6-Dioxo-3-piperidyl]-1,3-dioxo-isoindolin-4-yl]amino]hexyl]-4-piperidyl]-6-[[2-[(3R,4S)(3 S,4R)-3-fluoro-4-methoxy-1-piperidyl]pyrimidin-4-yl]amino]-1-isopropyl-pyrrolo[3,2-c]pyridine-3-carboxamide

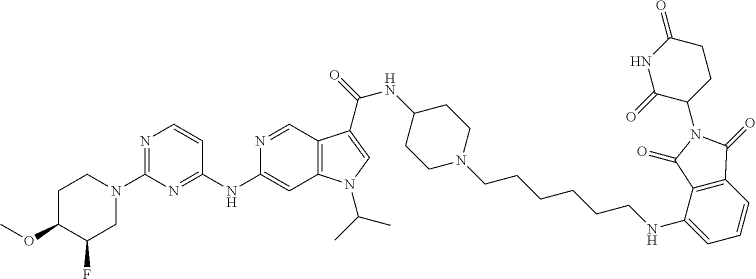 [SMILES: CO[C@H]1CCN(c2nccc(Nc3cc4c(cn3)c(C(=O)NC3CCN(CCCCCCNc5cccc6c5C(=O)N(C5CCC(=O)NC5=O)C6=O)CC3)cn4C(C)C)n2)C[C@H]1F]

The title compound was obtained as a yellow solid, MS: m/e=866.8 (M+H⁺), using chemistry similar to that described in Example 2, step 3 starting from 6-((2-((3R,4S)(3S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride (Example 5, step 2) and 4-(6-bromohexylamino)-2-[(3RS)-2,6-dioxo-3-piperidyl]isoindoline-1,3-dione (Example 2, step 2).

### Example 6

### (3RS)—N-[1-[6-[[2-[(3RS)-2,6-Dioxo-3-piperidyl]-1,3-dioxo-isoindolin-4-yl]amino]hexyl]-4-piperidyl]-1-[1-isopropyl-6-[[2-(4-methoxy-1-piperidyl)pyrimidin-4-yl]amino]pyrazolo[4,3-c]pyridin-3-yl]pyrrolidine-3-carboxamide

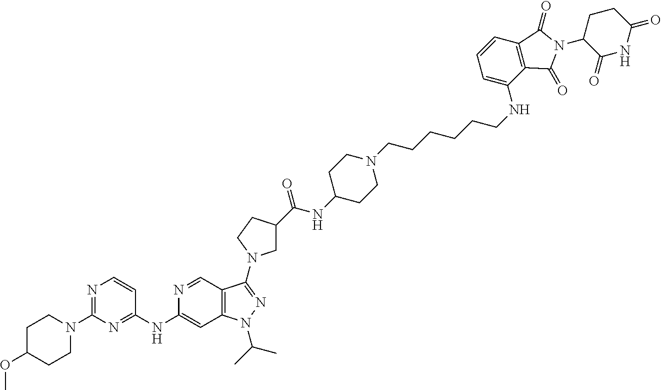 [SMILES: COC1CCN(c2nccc(Nc3cc4c(cn3)c(N3CCC(C(=O)NC5CCN(CCCCCCNc6cccc7c6C(=O)N(C6CCC(=O)NC6=O)C7=O)CC5)C3)nn4C(C)C)n2)CC1]

### Step 1: Methyl (3RS)-1-(6-bromo-1-isopropyl-pyrazolo[4,3-c]pyridin-3-yl)pyrrolidine-3-carboxylate

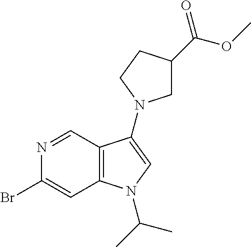 [SMILES: COC(=O)C1CCN(c2cn(C(C)C)c3cc(Br)ncc23)C1]

The title compound was obtained as a white solid, MS: m/e=367.5/369.5 (M+H⁺), using chemistry similar to that described in Example 1, step 3 starting from 6-bromo-3-iodo-1-isopropyl-1H-pyrazolo[4,3-c]pyridine (CAS 1639050-72-8) and methyl (3RS)-pyrrolidine-3-carboxylate hydrochloride.

### Step 2: Methyl (3RS)-1-[1-isopropyl-6-[[2-(4-methoxy-1-piperidyl)pyrimidin-4-yl]amino]pyrazolo[4,3-c]pyridin-3-yl ]pyrrolidine-3-carboxylate

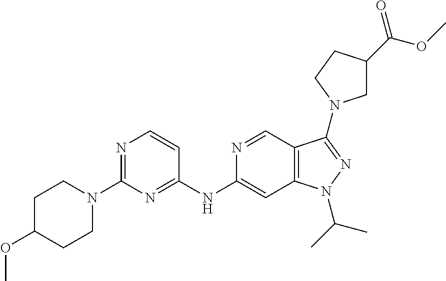 [SMILES: COC(=O)C1CCN(c2nn(C(C)C)c3cc(Nc4ccnc(N5CCC(OC)CC5)n4)ncc23)C1]

The title compound was obtained as a light brown solid, MS: m/e=495.7 (M+H⁺), using chemistry similar to that described in Example 1, step 3 starting from methyl (3RS)-1-(6-bromo-1-isopropyl-pyrazolo[4,3-c]pyridin-3-yl)pyrrolidine-3-carboxylate (Example 6, step 1) and 2-(4-methoxypiperidin-1-yl)pyrimidin-4-amine (Example 1, step 1).

### Step 3: (3RS)-1-[1-Isopropyl-6-[[2-(4-methoxy-1-piperidyl)pyrimidin-4-yl]amino]pyrazolo[4,3-c]pyridin-3-yl]-N-(4-piperidyl)pyrrolidine-3-carboxamide hydrochloride

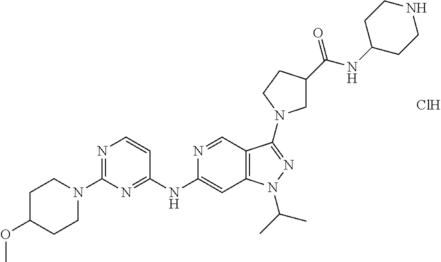 [SMILES: COC1CCN(c2nccc(Nc3cc4c(cn3)c(N3CCC(C(=O)NC5CCNCC5)C3)nn4C(C)C)n2)CC1 ; Cl]

The title compound was obtained as a yellow foam, MS: m/e=563.8 (M+H⁺), using chemistry similar to that described in Example 1, step 4 to 6 starting from methyl (3RS)-1-[1-isopropyl-6-[[2-(4-methoxy-1-piperidyl)pyrimidin-4-yl]amino]pyrazolo[4,3-c]pyridin-3-yl]pyrrolidine-3-carboxylate (Example 6, step 2).

### Step 4: (3RS)—N-[1-[6-[[2-[(3RS)-2,6-Dioxo-3-piperidyl]-1,3-dioxo-isoindolin-4-yl]amino]hexyl]-4-piperidyl]-1-[1-isopropyl-6-[[2-(4-methoxy-1-piperidyl)pyrimidin-4-yl]amino]pyrazolo[4,3-c]pyridin-3-yl]pyrrolidine-3-carboxamide

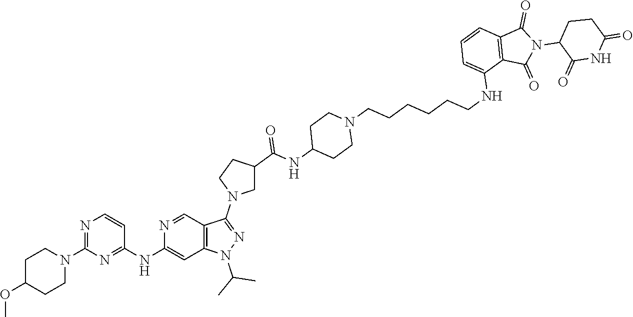 [SMILES: COC1CCN(c2nccc(Nc3cc4c(cn3)c(N3CCC(C(=O)NC5CCN(CCCCCCNc6cccc7c6C(=O)N(C6CCC(=O)NC6=O)C7=O)CC5)C3)nn4C(C)C)n2)CC1]

The title compound was obtained as a yellow solid, MS: m/e=919.0 (M+H⁺), using chemistry similar to that described in Example 2, step 3 starting from (3RS)-1-[l-isopropyl-6-[[2-(4-methoxy-1-piperidyl)pyrimidin-4-yl]amino]pyrazolo[4,3-c]pyridin-3-yl]-N-(4-piperidyl)pyrrolidine-3-carboxamide hydrochloride (Example 6, step 3) and 4-(6-bromohexylamino)-2-[(3RS)-2,6-dioxo-3-piperidyl]isoindoline-1,3-dione (Example 2, step 2).

### Example 7

### N-[1-[6-[[2-[(3RS)-2,6-Dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide, or

### N-[1-[6-[[2-[(3RS)-2,6-dioxo-3-piperidyl]-1,3-dioxo-isoindolin-4-yl]amino]hexyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide

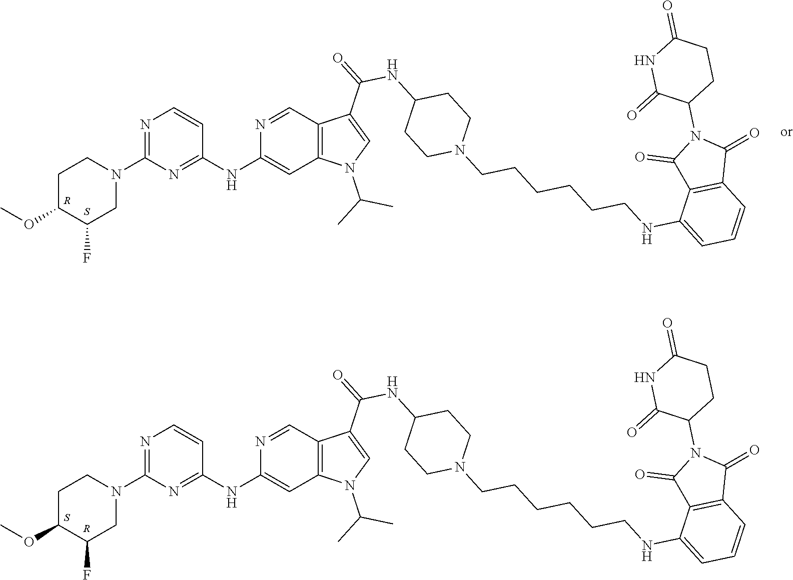

### Step 1: 2-((3R,4S)-3-Fluoro-4-methoxypiperidin-1-yl)pyrimidin-4-amine or 2-((3S,4R)-3-fluoro-4-methoxypiperidin-1-yl)pyrimidin-4-amine

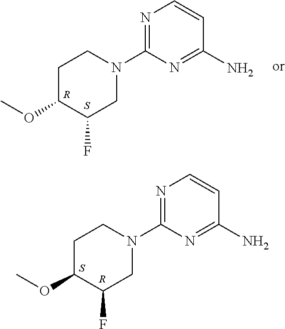

The title compound was obtained as a white solid, MS: m/e=227.3 (M+H⁺), separating 2-((3R,4S)(3 S,4R)-3-fluoro-4-methoxypiperidin-1-yl)pyrimidin-4-amine (Example 5, step 1) by SFC (Chiralcel OD-3 100×4.6 mm column with i-propanol with 0.05% of diethylamine in CO2 from 5% to 40%, Flow rate: 3 ml/min, Wavelength: 220 nm) and collecting peak B.

### Step 2: 6-((2-((3R,4S)-3-Fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride or 6-((2-((3 S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride

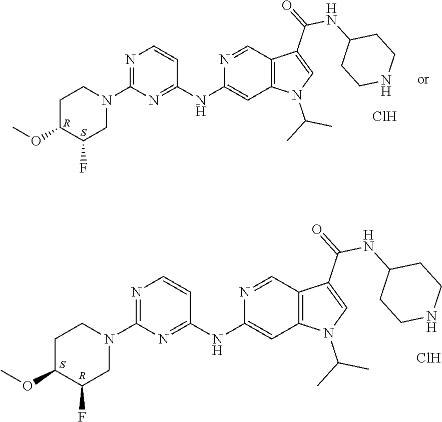

The title compound was obtained as a white solid, MS: m/e=511.7 (M+H⁺), using chemistry similar to that described in Example 1, step 3 to step 6 starting from methyl 6-chloro-1-isopropyl-1H-pyrrolo[3,2-c]pyridine-3-carboxylate (Example 1, step 2) and 2-((3R,4S)-3-fluoro-4-methoxypiperidin-1-yl)pyrimidin-4-amine or 2-((3 S,4R)-3-fluoro-4-methoxypiperidin-1-yl)pyrimidin-4-amine (Example 7, step 1).

### Step 3: N-[1-[4-[4-[2-[(3RS)-2,6-Dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide or N-[1-[6-[[2-[(3RS)-2,6-dioxo-3-piperidyl]-1,3-dioxo-isoindolin-4-yl]amino]hexyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide

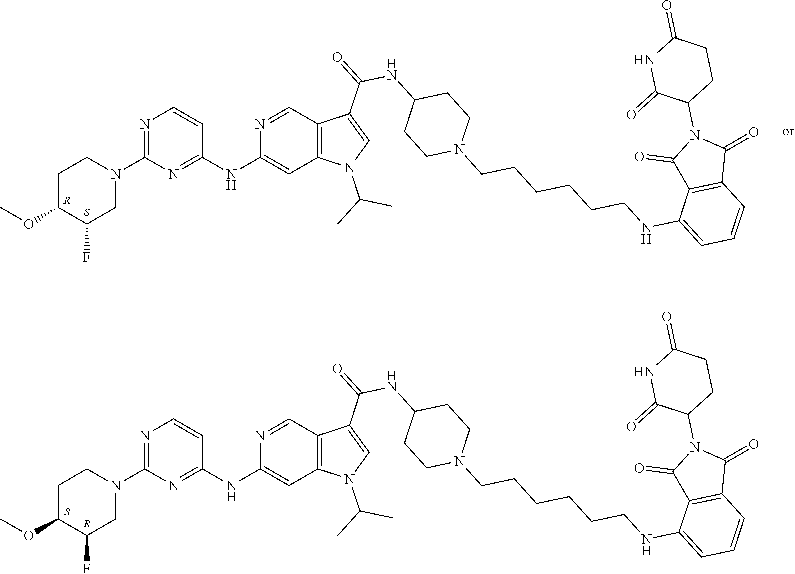

The title compound was obtained as a yellow solid, MS: m/e=866.4 (M+H⁺), using chemistry similar to that described in Example 2, step 3 starting from 6-((2-((3R,4S)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride or 6-((2-((3 S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride (Example 7, step 2) and 4-(6-bromohexylamino)-2-[(3RS)-2,6-dioxo-3-piperidyl]isoindoline-1,3-dione (Example 2, step 2).

### Example 8

### N-[1-[6-[[2-[(3RS)-2,6-Dioxo-3-piperidyl]-1,3-dioxo-isoindolin-4-yl]amino]hexyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide, or

### N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide

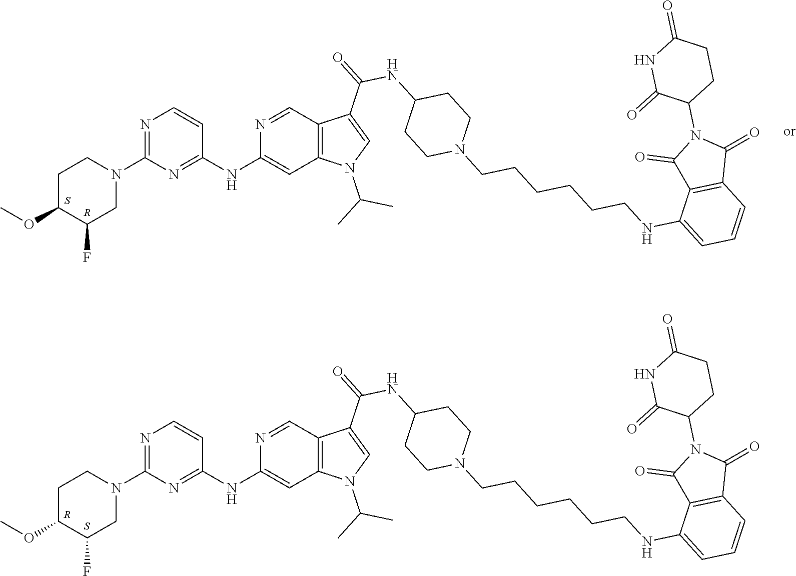

### Step 1: 2-((3S,4R)-3-Fluoro-4-methoxypiperidin-1-yl)pyrimidin-4-amine or 2-((3R,4S)-3-fluoro-4-methoxypiperidin-1-yl)pyrimidin-4-amine

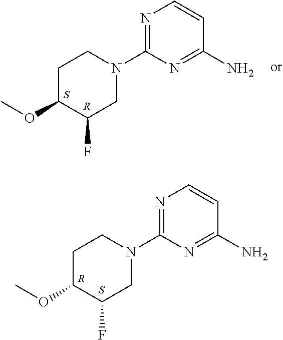

The title compound was obtained as a white solid, MS: m/e=227.3 (M+H⁺), separating 2-((3R,4S)(3 S,4R)-3-fluoro-4-methoxypiperidin-1-yl)pyrimidin-4-amine (Example 5, step 1) by SFC (Chiralcel OD-3 100×4.6 mm column with i-propanol with 0.05% of diethylamine in CO2 from 5% to 40%, Flow rate: 3 ml/min, Wavelength: 220 nm) and collecting peak A.

### Step 2: 6-((2-((3 S,4R)-3-Fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride or 6-((2-((3R,4 S)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride

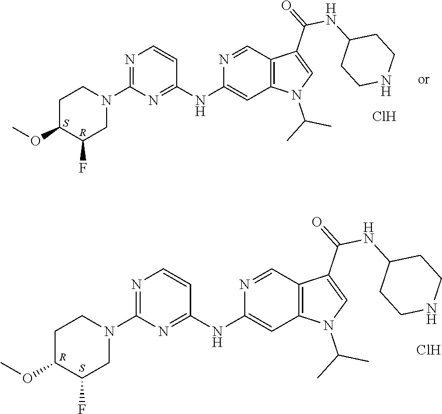

The title compound was obtained as a white solid, MS: m/e=511.2 (M+H⁺), using chemistry similar to that described in Example 1, step 3 to step 6 starting from methyl 6-chloro-1-isopropyl-1H-pyrrolo[3,2-c]pyridine-3-carboxylate (Example 1, step 2) and 2-((3 S,4R)-3-fluoro-4-methoxypiperidin-1-yl)pyrimidin-4-amine or 2-((3R,4S)-3-fluoro-4-methoxypiperidin-1-yl)pyrimidin-4-amine (Example 8, step 1).

### Step 3: N-[1-[6-[[2-[(3RS)-2,6-Dioxo-3-piperidyl]-1,3-dioxo-isoindolin-4-yl]amino]hexyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide or N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide

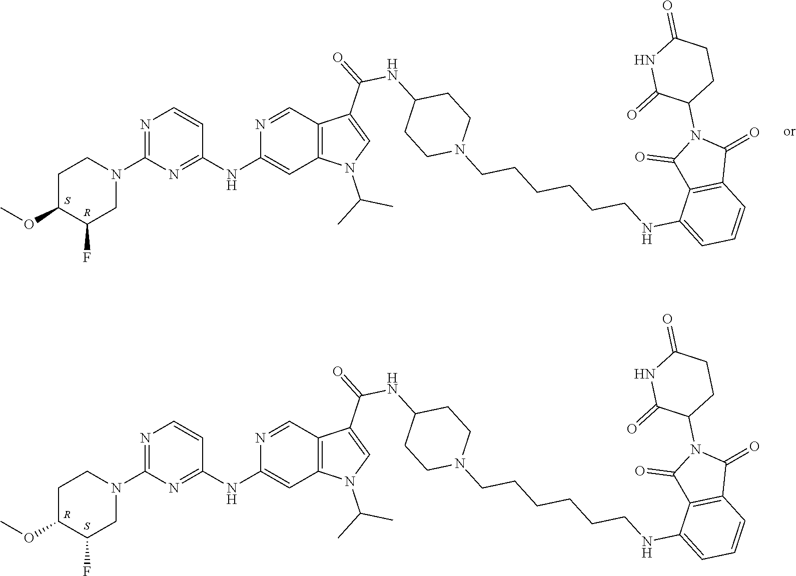

The title compound was obtained as a yellow solid, MS: m/e=866.8 (M+H⁺), using chemistry similar to that described in Example 2, step 3 starting from 6-((2-((3S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride or 6-((2-((3R,4 S)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride (Example 8, step 2) and 4-(6-bromohexylamino)-2-[(3RS)-2,6-dioxo-3-piperidyl]isoindoline-1,3-dione (Example 2, step 2).

### Example 9

### N-[1-[5-[[2-[(3RS)-2,6-Dioxo-3-piperidyl]-1,3-dioxo-isoindolin-4-yl]amino]pentyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide, or

### N-[1-[5-[[2-[(3RS)-2,6-dioxo-3-piperidyl]-1,3-dioxo-isoindolin-4-yl]amino]pentyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide

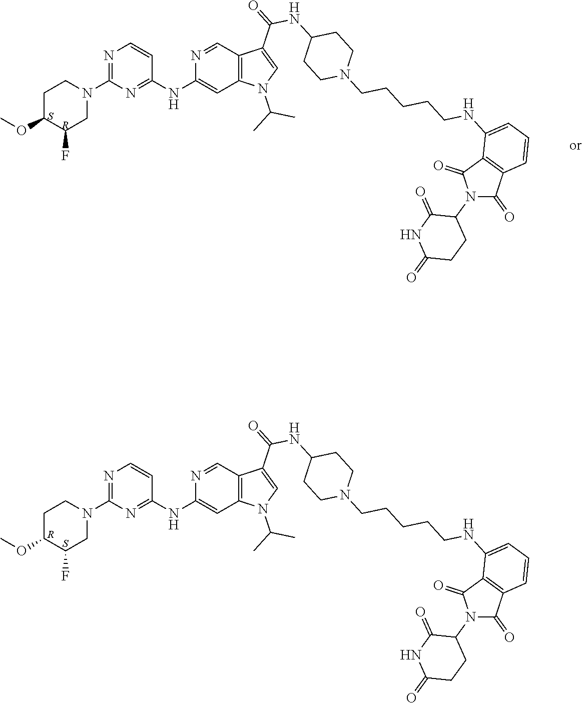

### Step 1: 4-(5-Bromopentylamino)-2-[(3RS)-2,6-dioxo-3-piperidyl]isoindoline-1,3-dione

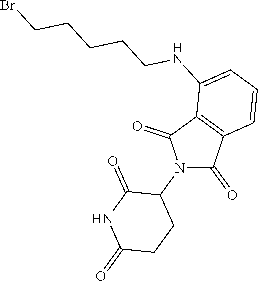 [SMILES: O=C1CCC(N2C(=O)c3cccc(NCCCCCBr)c3C2=O)C(=O)N1]

The title compound was obtained as a yellow foam, MS: m/e=421.9/423.9 (M+H⁺), using chemistry similar to that described in Example 2, step 1 and step 2 starting from 2-[(3RS)-2,6-dioxo-3-piperidyl]-4-fluoro-isoindoline-1,3-dione (CAS 835616-60-9) and 5-aminopentan-1-ol.

### Step 2: N-[1-[5-[[2-[(3RS)-2,6-Dioxo-3-piperidyl]-1,3-dioxo-isoindolin-4-yl]amino]pentyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide or N-[1-[5-[[2-[(3RS)-2,6-dioxo-3-piperidyl]-1,3-dioxo-isoindolin-4-yl]amino]pentyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide

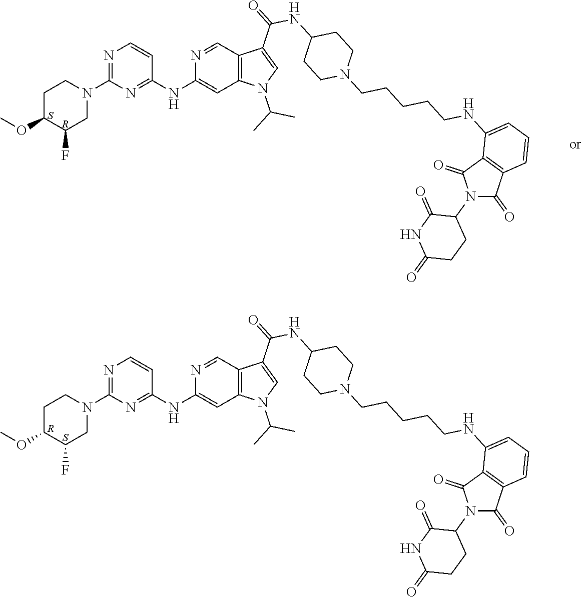

The title compound was obtained as a yellow foam, MS: m/e=852.6 (M+H⁺), using chemistry similar to that described in Example 2, step 3 starting from 6-((2-((3S,4R)-3-fluoro-4-methoxypiperidin-l-yl)pyrimidin-4-yl)amino)-1-isopropyl-N-(piperidin-4-yl)-1H-pyrrolo[3,2-c]pyridine-3-carboxamide hydrochloride or 6-((2-((3R,4S)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride (Example 8, step 2) and 4-(5-bromopentylamino)-2-[(3RS)-2,6-dioxo-3-piperidyl]isoindoline-1,3-dione (Example 9, step 1).

### Example 10

### 1-Cyclopentyl-N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide, or

### 1-cyclopentyl-N-[1-[6-[[2-[(3RS)-2,6-dioxo-3-piperidyl]-1,3-dioxo-isoindolin-4-yl]amino]hexyl]-4-piperidyl]-6-[[2-[(3S,4R)-3-fluoro-4-methoxy-1-piperidyl]pyrimidin-4-yl]amino]pyrrolo[3,2-c]pyridine-3-carboxamide

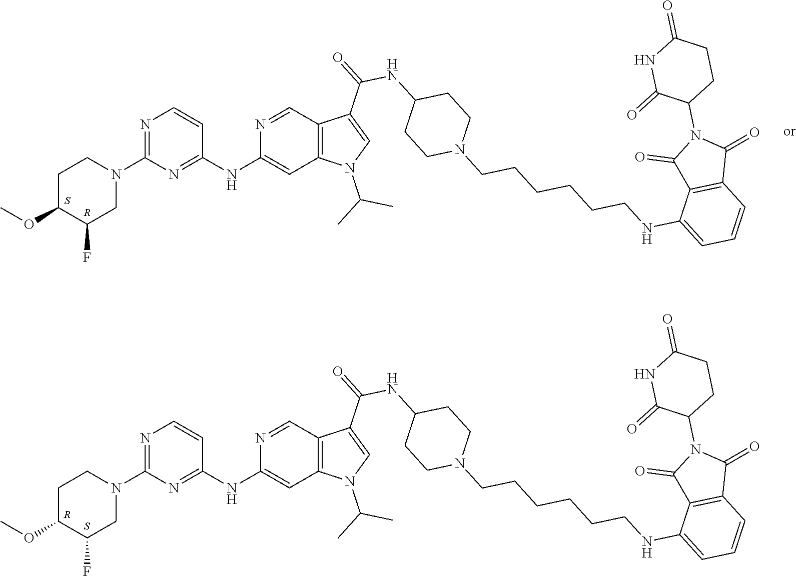

### Step 1: 1-Cyclopentyl-6-[[2-[(3R,4 S)-3-fluoro-4-methoxy-1-piperidyl]pyrimidin-4-yl]amino]-N-(4-piperidyl)pyrrolo[3,2-c]pyridine-3-carboxamide hydrochloride or 1-cyclopentyl-6-[[2-[(3 S,4R)-3-fluoro-4-methoxy-1-piperidyl]pyrimidin-4-yl]amino]-N-(4-piperidyl)pyrrolo[3,2-c]pyridine-3-carboxamide hydrochloride

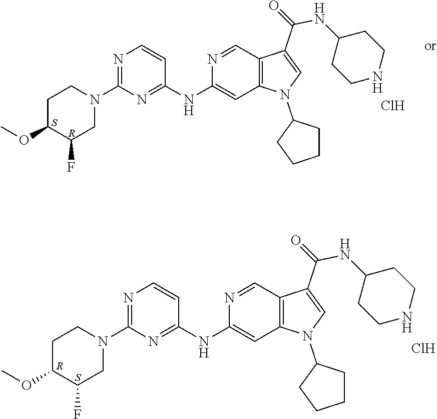

The title compound was obtained as a white foam, MS: m/e=537.5 (M+H⁺), using chemistry similar to that described in Example 1, step 3 to step 6 starting from methyl 6-chloro-1-cyclopentyl-1H-pyrrolo[3,2-c]pyridine-3-carboxylate (Example 4, step 1) and 2-((3 S,4R)-3-fluoro-4-methoxypiperidin-1-yl)pyrimidin-4-amine or 2-((3R,4 S)-3-fluoro-4-methoxypiperidin-1-yl)pyrimidin-4-amine (Example 8, step 1).

### Step 2: 1-Cyclopentyl-N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide or 1-cyclopentyl-N-[1-[6-[[2-[(3RS)-2,6-dioxo-3-piperidyl]-1,3-dioxo-isoindolin-4-yl]amino]hexyl]-4-piperidyl]-6-[[2-[(3S,4R)-3-fluoro-4-methoxy-1-piperidyl]pyrimidin-4-yl]amino]pyrrolo[3,2-c]pyridine-3-carboxamide

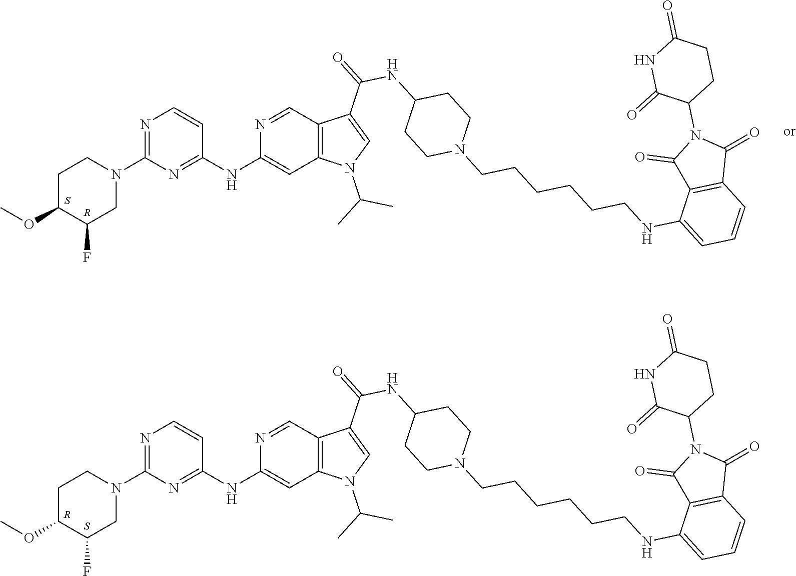

The title compound was obtained as a yellow solid, MS: m/e=892.8 (M+H⁺), using chemistry similar to that described in Example 2, step 3 starting from 1-cyclopentyl-6-[[2-[(3R,4S)-3-fluoro-4-methoxy-1-piperidyl]pyrimidin-4-yl]amino]-N-(4-piperidyl)pyrrolo[3,2-c]pyridine-3-carboxamide hydrochloride or 1-cyclopentyl-6-[[2-[(3S,4R)-3-fluoro-4-methoxy-1-piperidyl]pyrimidin-4-yl]amino]-N-(4-piperidyl)pyrrolo[3,2-c]pyridine-3-carboxamide hydrochloride (Example 10, step 1) and 4-(6-bromohexylamino)-2-[(3RS)-2,6-dioxo-3-piperidyl]isoindoline-1,3-dione (Example 2, step 2).

### Example 11

### 1-Cyclopentyl-N-[1-[5-[[2-[(3RS)-2,6-dioxo-3-piperidyl]-1,3-dioxo-isoindolin-4-yl]amino]pentyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide, or

### 1-cyclopentyl-N-[1-[5-[[2-[(3RS)-2,6-dioxo-3-piperidyl]-1,3-dioxo-isoindolin-4-yl]amino]pentyl]-4-piperidyl]-6-[[2-[(3S,4R)-3-fluoro-4-methoxy-1-piperidyl]pyrimidin-4-yl]amino]pyrrolo[3,2-c]pyridine-3-carboxamide

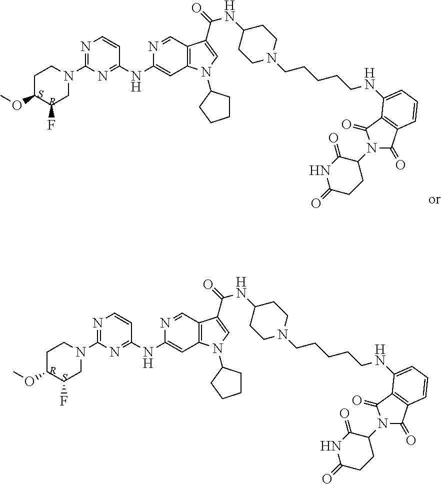

The title compound was obtained as a yellow solid, MS: m/e=876.9 (M−H⁺), using chemistry similar to that described in Example 2, step 3 starting from 1-cyclopentyl-6-[[2-[(3R,4 S)-3-fluoro-4-methoxy-1-piperidyl]pyrimidin-4-yl]amino]-N-(4-piperidyl)pyrrolo[3,2-c]pyridine-3-carboxamide hydrochloride or 1-cyclopentyl-6-[[2-[(3S,4R)-3-fluoro-4-methoxy-1-piperidyl]pyrimidin-4-yl]amino]-N-(4-piperidyl)pyrrolo[3,2-c]pyridine-3-carboxamide hydrochloride (Example 10, step 1) and 4-(5-bromopentylamino)-2-[(3RS)-2,6-dioxo-3-piperidyl]isoindoline-1,3-dione (Example 9, step 1).

### Example 12

### N-[1-[3-[1-[2-[(3RS)-2,6-Dioxo-3-piperidyl]-1,3-dioxo-isoindolin-4-yl]-4-piperidyl]propyl]-4-piperidyl]-6-[[2-[(3R,4S)(3S,4R)-3-fluoro-4-methoxy-1-piperidyl]pyrimidin-4-yl]amino]-1-isopropyl-pyrrolo[3,2-c]pyridine-3-carboxamide trifluoroacetate

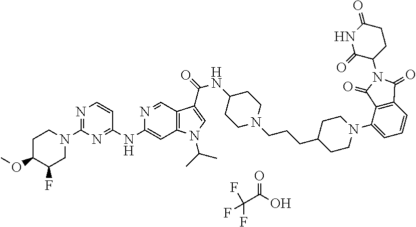 [SMILES: CO[C@H]1CCN(c2nccc(Nc3cc4c(cn3)c(C(=O)NC3CCN(CCCC5CCN(c6cccc7c6C(=O)N(C6CCC(=O)NC6=O)C7=O)CC5)CC3)cn4C(C)C)n2)C[C@H]1F ; O=C(O)C(F)(F)F]

### Step 1: 2-(2,6-Dioxopiperidin-3-yl)-4-(4-(3-hydroxypropyl)piperidin-1-yl)isoindoline-1,3-dione

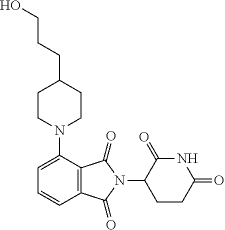 [SMILES: O=C1CCC(N2C(=O)c3cccc(N4CCC(CCCO)CC4)c3C2=O)C(=O)N1]

The title compound was obtained as a yellow foam, MS: m/e=400.9 (M+H⁺), using chemistry similar to that described in Example 1, step 7 starting from 2-[(3RS)-2,6-dioxo-3-piperidyl]-4-fluoro-isoindoline-1,3-dione (CAS 835616-60-9) and 3-(4-piperidyl)propan-1-ol.

### Step 2: N-[1-[3-[1-[2-[(3RS)-2,6-Dioxo-3-piperidyl]-1,3-dioxo-isoindolin-4-yl]-4-piperidyl]propyl]-4-piperidyl]-6-[[2-[(3R,4S)(3 S,4R)-3-fluoro-4-methoxy-1-piperidyl]pyrimidin-4-yl]amino]-1-isopropyl-pyrrolo[3,2-c]pyridine-3-carboxamide trifluoroacetate

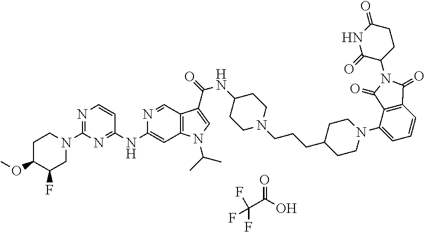 [SMILES: CO[C@H]1CCN(c2nccc(Nc3cc4c(cn3)c(C(=O)NC3CCN(CCCC5CCN(c6cccc7c6C(=O)N(C6CCC(=O)NC6=O)C7=O)CC5)CC3)cn4C(C)C)n2)C[C@H]1F ; O=C(O)C(F)(F)F]

2-(2,6-Dioxo-3-piperidyl)-4-[4-(3-hydroxypropyl)-1-piperidyl]isoindoline-1,3-dione (Example 12, step 1) (37.55 mg, 94.01 μmol) was dissolved in DCM (1 mL) and TEA (16.38 μL, 117.51 μmol, 1.25 equiv.) was added, followed by methanesulfonyl chloride (7.28 μL, 94.01 μmol, 1 equiv.) dropwise. The reaction mixture was stirred for 30 minutes and concentrated to dryness. DMAc (0.5 mL) was added to the residue and 6-[[2-[(3R,4S)(3S,4R)-3-fluoro-4-methoxy-1-piperidyl]pyrimidin-4-yl]amino]-1-isopropyl-N-(4-piperidyl)pyrrolo[3,2-c]pyridine-3-carboxamide (Example 5, step 2) (40 mg, 78.34 μmol) was added, followed by DIEA (68.23 μL, 391.69 μmol, 4.2 equiv.) and potassium iodide (26.01 mg, 156.68 μmol, 1.65 equiv.). The mixture was stirred at 80° C. for 18 hours. The mixture was purified by flash column chromatography on silica gel (0-20% 1.75 M ammonia in MeOH in DCM), followed by reverse-phase chromatography on 50 g C18 column (5%-100% acetonitrile in water with 0.1% TFA as modifier) to give N-[1-[3-[1-[2-[(3RS)-2,6-dioxo-3-piperidyl]-1,3-dioxo-isoindolin-4-yl]-4-piperidyl]propyl]-4-piperidyl]-6-[[2-[(3R,4S)(3 S,4R)-3-fluoro-4-methoxy-1-piperidyl]pyrimidin-4-yl]amino]-1-isopropyl-pyrrolo[3,2-c]pyridine-3-carboxamide trifluoroacetate (42.4 mg, 54%) as a yellow solid. MS: m/e=892.7 (M+H⁺).

### Example 13

### 1-Cyclopentyl-N-[1-[4-[4-[[2-[(3RS)-2,6-dioxo-3-piperidyl]-1,3-dioxo-isoindolin-4-yl]amino]butyl]piperazine-1-carbonyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide, or

### 1-cyclopentyl-N-[1-[4-[4-[[2-[(3RS)-2,6-dioxo-3-piperidyl]-1,3-dioxo-isoindolin-4-yl]amino]butyl]piperazine-1-carbonyl]-4-piperidyl]-6-[[2-[(3S,4R)-3-fluoro-4-methoxy-1-piperidyl]pyrimidin-4-yl]amino]pyrrolo[3,2-c]pyridine-3-carboxamide

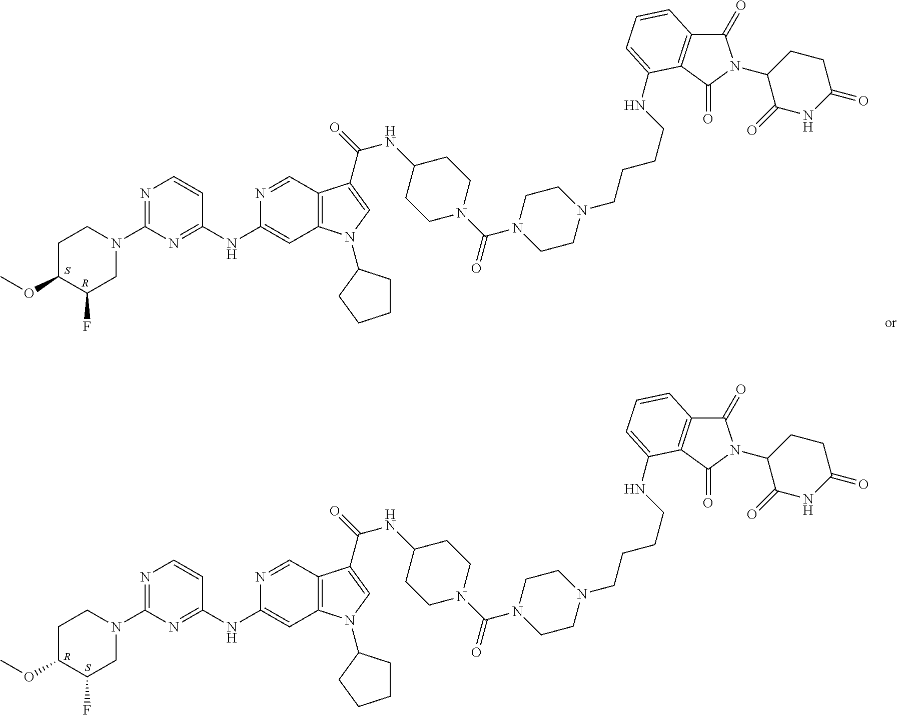

### Step 1: tert-Butyl 4-[4-[[2-[(3RS)-2,6-dioxo-3-piperidyl]-1,3-dioxo-isoindolin-4-yl]amino]butyl]piperazine-1-carboxylate

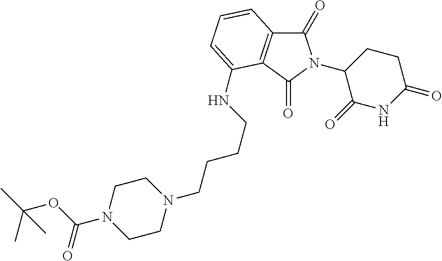 [SMILES: CC(C)(C)OC(=O)N1CCN(CCCCNc2cccc3c2C(=O)N(C2CCC(=O)NC2=O)C3=O)CC1]

The title compound was obtained as a yellow solid, MS: m/e=514.5 (M+H⁺), using chemistry similar to that described in Example 1, step 7 starting from 2-[(3RS)-2,6-dioxo-3-piperidyl]-4-fluoro-isoindoline-1,3-dione (CAS 835616-60-9) and tert-butyl 4-(4-aminobutyl)piperazine-1-carboxylate, by using NMP instead of DMSO as solvent.

### Step 2: 2-[(3RS)-2,6-Dioxo-3-piperidyl]-4-(4-piperazin-1-ylbutylamino)isoindoline-1,3-dione hydrochloride

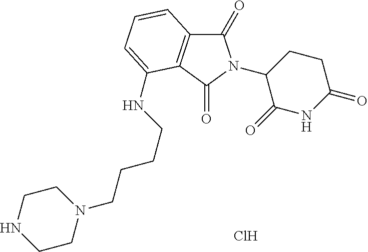 [SMILES: O=C1CCC(N2C(=O)c3cccc(NCCCCN4CCNCC4)c3C2=O)C(=O)N1 ; Cl]

The title compound was obtained as a yellow solid, MS: m/e=414.4 (M+H⁺), using chemistry similar to that described in Example 1, step 6 starting from tert-butyl 4-[4-[[2-[(3RS)-2,6-dioxo-3-piperidyl]-1,3-dioxo-isoindolin-4-yl]amino]butyl]piperazine-1-carboxylate (Example 13, step 1).

### Step 3: 1-Cyclopentyl-N-[1-[4-[4-[[2-[(3RS)-2,6-dioxo-3-piperidyl]-1,3-dioxo-isoindolin-4-yl]amino]butyl]piperazine-1-carbonyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide or 1-cyclopentyl-N-[1-[4-[4-[[2-[(3RS)-2,6-dioxo-3-piperidyl]-1,3-dioxo-isoindolin-4-yl]amino]butyl]piperazine-1-carbonyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide

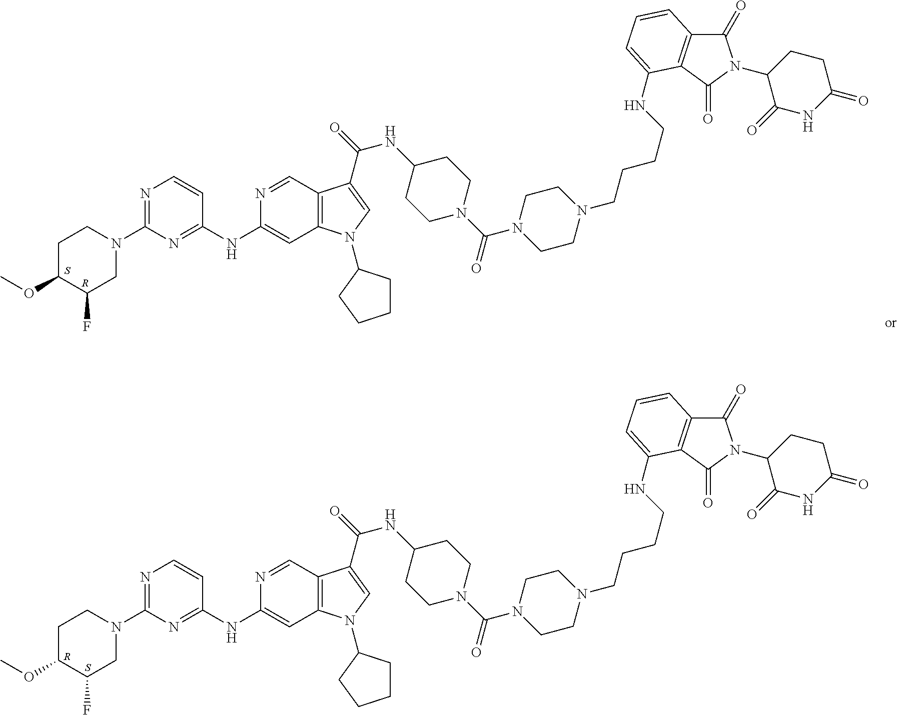

Triphosgene (11 mg, 0.036 mmol, 0.35 equiv.) was dissolved in 0.5 ml of dichloromethane and cooled to 0-5° C. A solution of 1-cyclopentyl-6-[[2-[(3R,4S)-3-fluoro-4-methoxy-1-piperidyl]pyrimidin-4-yl]amino]-N-(4-piperidyl)pyrrolo[3,2-c]pyridine-3-carboxamide or 1-cyclopentyl-6-[[2-[(3 S,4R)-3-fluoro-4-methoxy-1-piperidyl]pyrimidin-4-yl]amino]-N-(4-piperidyl)pyrrolo[3,2-c]pyridine-3-carboxamide (Example 10, step 1, as free base) (55 mg, 0.10 mmol) in 3 ml of dichloromethane was added and stirred for 10 minutes at 0-5° C. A mixture of 2-[(3RS)-2,6-dioxo-3-piperidyl]-4-(4-piperazin-1-ylbutylamino)isoindoline-1,3-dione hydrochloride (Example 13, step 2) (46 mg, 0.10 mmol, 1 equiv.) and Hunig's base (66 mg, 0.09 ml, 0.51 mmol, 5 equiv.) in 3 ml of dichloromethane was added at 0-5° C. The mixture was stirred at room temperature for 4 hours. The reaction mixture was extracted with water and three times with dichloromethane:methanol):1 mixture. The organic layers were dried over sodium sulfate and evaporated to dryness. The crude product was purified by flash chromatography on a silica gel column eluting with a dichloromethane:methanol 100:0 to 80:20 gradient to obtain the desired 1-cyclopentyl-N-[1-[4-[4-[[2-[(3RS)-2,6-dioxo-3-piperidyl]-1,3-dioxo-isoindolin-4-yl]amino]butyl]piperazine-1-carbonyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide or 1-cyclopentyl-N-[1-[4-[4-[[2-[(3RS)-2,6-dioxo-3-piperidyl]-1,3-dioxo-isoindolin-4-yl]amino]butyl]piperazine-1-carbonyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide (38 mg, 38% yield) as a yellow solid, MS: m/e=977.0 (M+H⁺).

### Example 14

### N-[1-[4-[[2-[(3RS)-2,6-Dioxo-3-piperidyl]-1,3-dioxo-isoindolin-4-yl]amino]butyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide, or

### N-[1-[4-[[2-[(3RS)-2,6-dioxo-3-piperidyl]-1,3-dioxo-isoindolin-4-yl]amino]butyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide

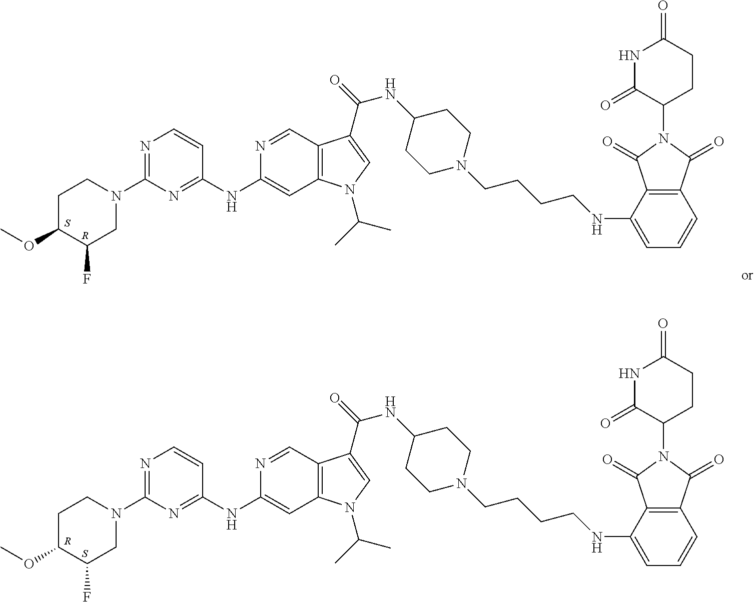

### Step 1: 4-(4-Bromobutylamino)-2-[(3RS)-2,6-dioxo-3-piperidyl]isoindoline-1,3-dione

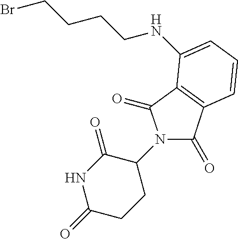 [SMILES: O=C1CCC(N2C(=O)c3cccc(NCCCCBr)c3C2=O)C(=O)N1]

The title compound was obtained as a dark green foam, MS: m/e=408.3/410.3 (M+H⁺), using chemistry similar to that described in Example 2, step 1 and step 2 starting from 2-[(3RS)-2,6-dioxo-3-piperidyl]-4-fluoro-isoindoline-1,3-dione (CAS 835616-60-9) and 4-aminobutan-1-ol.

### Step 2: N-[1-[4-[4-[2-[(3RS)-2,6-Dioxo-3-piperidyl]-1,3-dioxo-isoindolin-4-yl]amino]butyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide or N-[1-[4-[[2-[(3RS)-2,6-dioxo-3-piperidyl]-1,3-dioxo-isoindolin-4-yl]amino]butyl]-4-piperidyl]-6-[[2-[(3 S,4R)-3-fluoro-4-methoxy-1-piperidyl]pyrimidin-4-yl]amino]-l-isopropyl-pyrrolo[3,2-c]pyridine-3-carboxamide

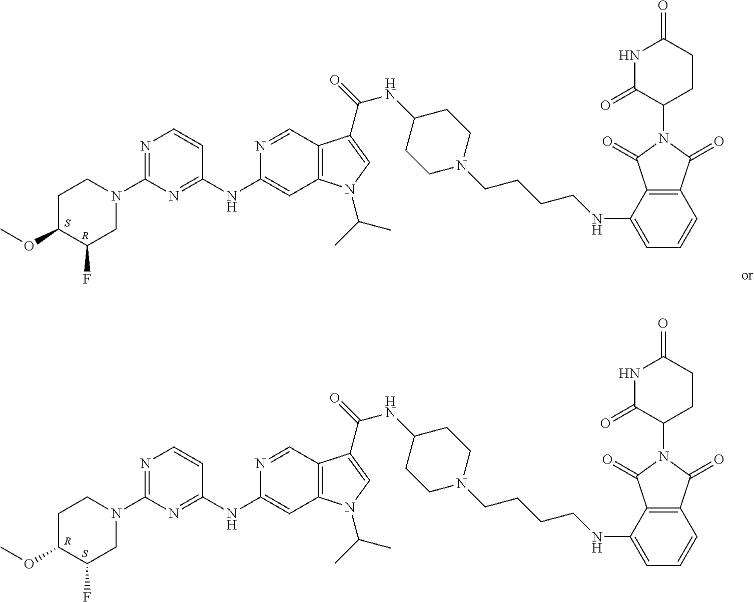

The title compound was obtained as a yellow foam, MS: m/e=864.8 (M+H⁺), using chemistry similar to that described in Example 2, step 3 starting from 6-((2-((3S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride or 6-((2-((3R,4S)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride (Example 8, step 2) and 4-(4-bromobutylamino)-2-[(3RS)-2,6-dioxo-3-piperidyl]isoindoline-1,3-dione (Example 14, step 1).

### Example 15

### 1-Cyclopentyl-N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide, or

1-cyclopentyl-N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3S,4R)-3-fluoro-4-methoxy-1-piperidyl]pyrimidin-4-yl]amino]pyrrolo[3,2-c]pyridine-3-carboxamide

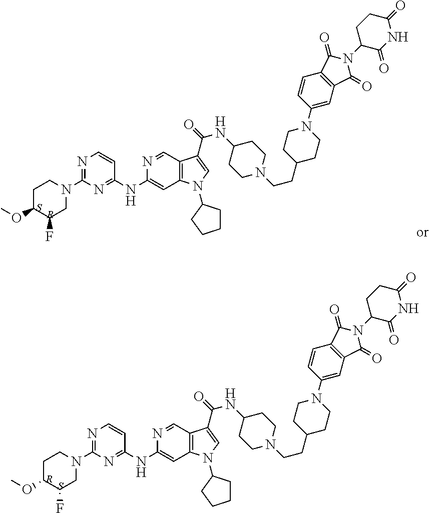

### Step 1: 5-[4-(2-Bromoethyl)-1-piperidyl]-2-[(3RS)-2,6-dioxo-3-piperidyl]isoindoline-1,3-dione

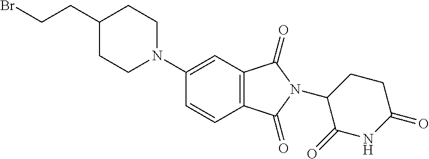 [SMILES: O=C1CCC(N2C(=O)c3ccc(N4CCC(CCBr)CC4)cc3C2=O)C(=O)N1]

The title compound was obtained as a brown solid, MS: m/e=448.1/450.1 (M+H⁺), using chemistry similar to that described in Example 2, step 1 and step 2 starting from 2-[(3RS)-2,6-dioxo-3-piperidyl]-5-fluoro-isoindoline-1,3-dione (CAS 835616-61-0) and 2-(piperidin-4-yl)ethan-1-ol.

### Step 2: 1-Cyclopentyl-N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide or 1-cyclopentyl-N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3S,4R)-3-fluoro-4-methoxy-1-piperidyl]pyrimidin-4-yl]amino]pyrrolo[3,2-c]pyridine-3-carboxamide

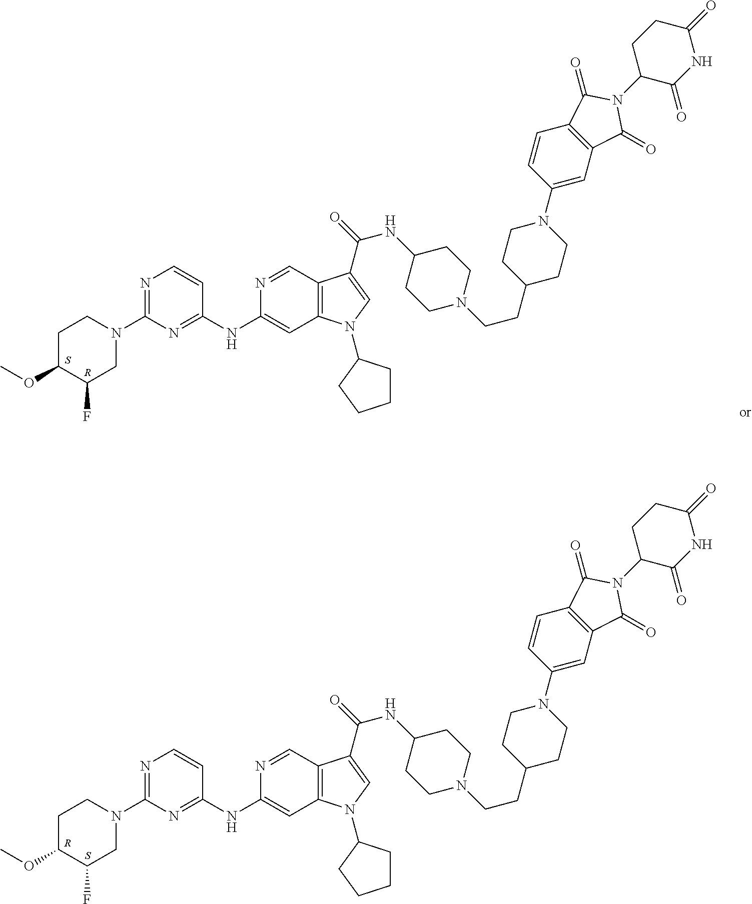

The title compound was obtained as a yellow solid, MS: m/e=904.7 (M-1-11, using chemistry similar to that described in Example 2, step 3 starting from 1-cyclopentyl-6-[[2-[(3R,4S)-3-fluoro-4-methoxy-1-piperidyl]pyrimidin-4-yl]amino]-N-(4-piperidyl)pyrrolo[3,2-c]pyridine-3-carboxamide hydrochloride or 1-cyclopentyl-6-[[2-[(3S,4R)-3-fluoro-4-methoxy-1-piperidyl]pyrimidin-4-yl]amino]-N-(4-piperidyl)pyrrolo[3,2-c]pyridine-3-carboxamide hydrochloride (Example 10, step 1) and 5-[4-(2-bromoethyl)-1-piperidyl]-2-[(3RS)-2,6-dioxo-3-piperidyl]isoindoline-1,3-dione (Example 15, step 1).

### Example 16

### N-[1-[2-[1-[2-[(3RS)-2,6-Dioxo-3-piperidyl]-1,3-dioxo-isoindolin-5-yl]-4-piperidyl]ethyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide or

### N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide

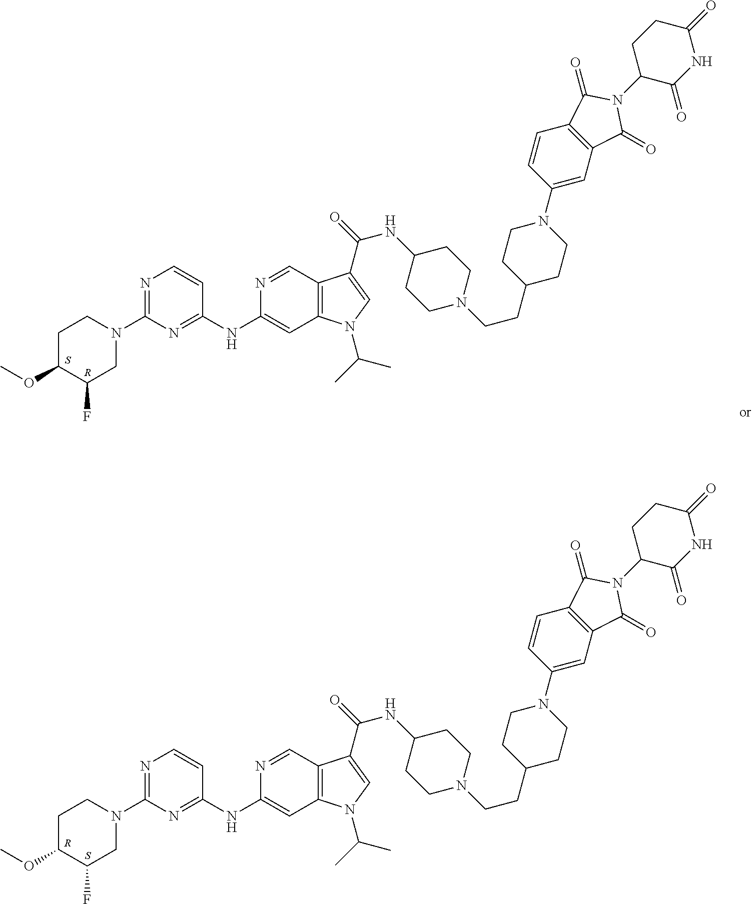

The title compound was obtained as a yellow solid, MS: m/e=878.8 (M−H⁺), using chemistry similar to that described in Example 2, step 3 starting from 6-((2-((3S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride or 6-((2-((3R,4S)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride (Example 8, step 2) and 5-[4-(2-bromoethyl)-1-piperidyl]-2-[(3RS)-2,6-dioxo-3-piperidyl]isoindoline-1,3-dione (Example 15, step 1).

### Example 17

### N-[1-[3-[[1-[2-[(3RS)-2,6-Dioxo-3-piperidyl]-1,3-dioxo-isoindolin-4-yl]-4-piperidyl]oxy]propyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide, or

### N-[1-[3-[[1-[2-[(3RS)-2,6-dioxo-3-piperidyl]-1,3-dioxo-isoindolin-4-yl]-4-piperidyl]oxy]propyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide

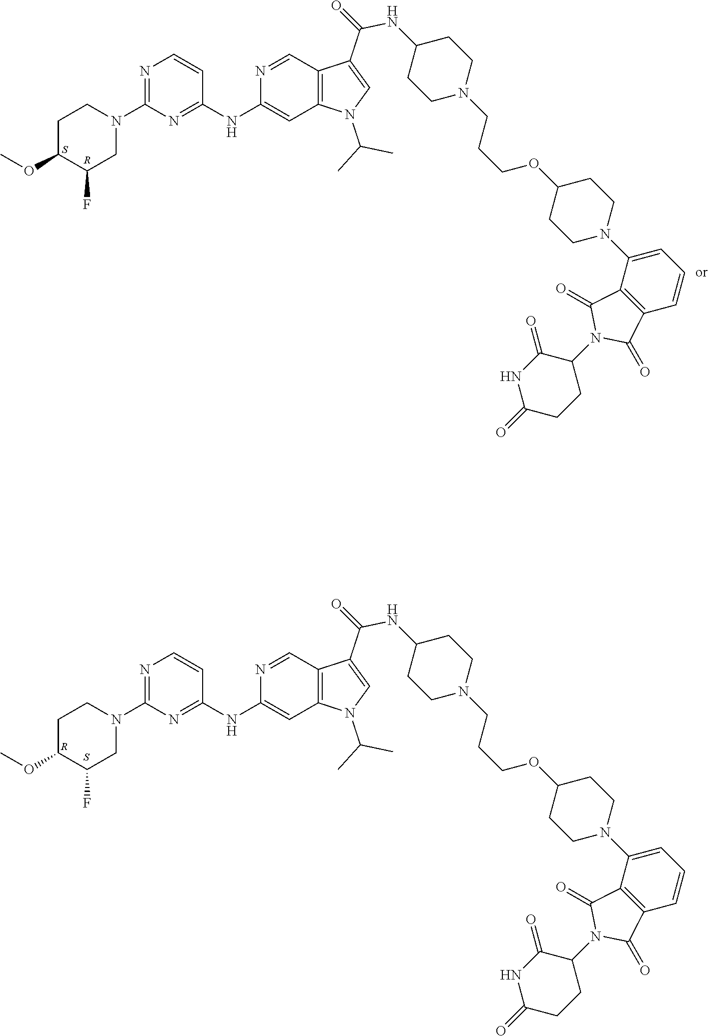

### Step 1: 4-[4-(3-Bromopropoxy)-1-piperidyl]-2-[(3RS)-2,6-dioxo-3-piperidyl]isoindoline-1,3-dione

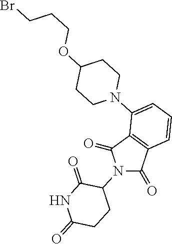 [SMILES: O=C1CCC(N2C(=O)c3cccc(N4CCC(OCCCBr)CC4)c3C2=O)C(=O)N1]

The title compound was obtained as a yellow solid, MS: m/e=478.3/480.3 (M+H⁺), using chemistry similar to that described in Example 2, step 1 and step 2 starting from 2-[(3RS)-2,6-dioxo-3-piperidyl]-4-fluoro-isoindoline-1,3-dione (CAS 835616-60-9) and 3-(piperidin-4-yloxy)propan-1-ol.

### Step 2: N-[1-[3-[[1-[2-[(3RS)-2,6-Dioxo-3-piperidyl]-1,3-dioxo-isoindolin-4-yl]-4-piperidyl]oxy]propyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide or N-[1-[3-[[1-[2-[(3RS)-2,6-dioxo-3-piperidyl]-1,3-dioxo-isoindolin-4-yl]-4-piperidyl]oxy]propyl]-4-piperidyl]-6-[[2-[(3 S,4R)-3-fluoro-4-methoxy-l-piperidyl]pyrimidin-4-yl]amino]-1-isopropyl-pyrrolo[3,2-c]pyridine-3-carboxamide

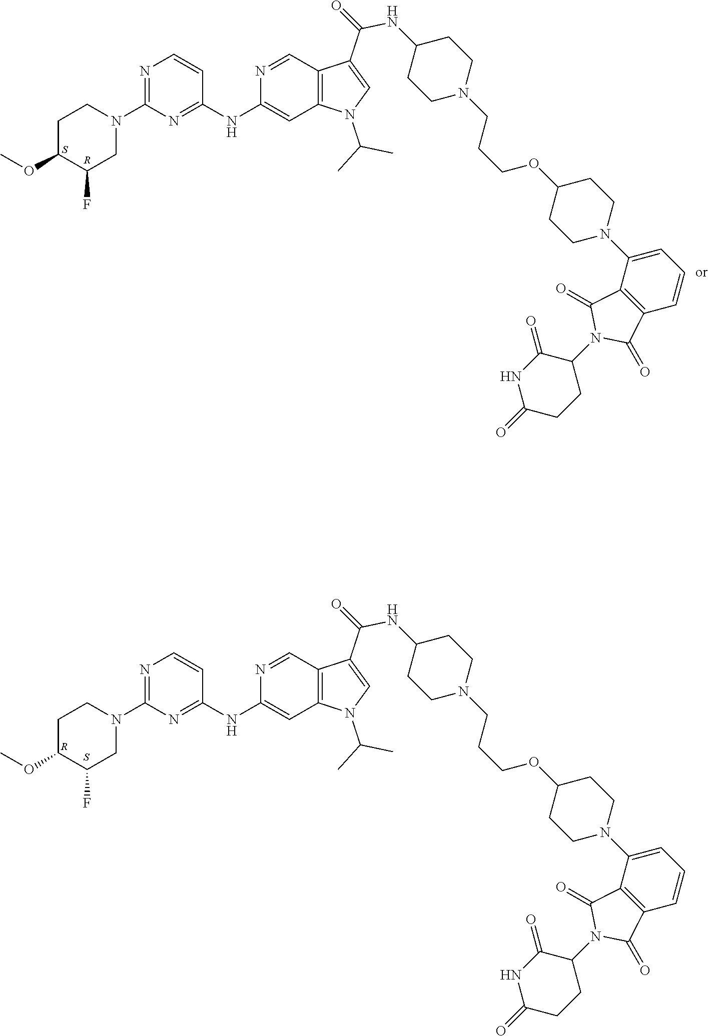

The title compound was obtained as a yellow solid, MS: m/e=908.9 (M+H⁺), using chemistry similar to that described in Example 2, step 3 starting from 6-((2-((3S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride or 6-((2-((3R,4S)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride (Example 8, step 2) and 4-[4-(3-bromopropoxy)-1-piperidyl]-2-[(3RS)-2,6-dioxo-3-piperidyl]isoindoline-1,3-dione (Example 17, step 1).

### Example 18

### 1-Cyclopentyl-N-[1-[3-[[1-[2-[(3S)-2,6-dioxo-3-piperidyl]-1,3-dioxo-isoindolin-4-yl]-4-piperidyl]oxy]propyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide, or

### 1-cyclopentyl-N-[1-[3-[[1-[2-[(3S)-2,6-dioxo-3-piperidyl]-1,3-dioxo-isoindolin-4-yl]-4-piperidyl]oxy]propyl]-4-piperidyl]-6-[[2-[(3S,4R)-3-fluoro-4-methoxy-1-piperidyl]pyrimidin-4-yl]amino]pyrrolo[3,2-c]pyridine-3-carboxamide

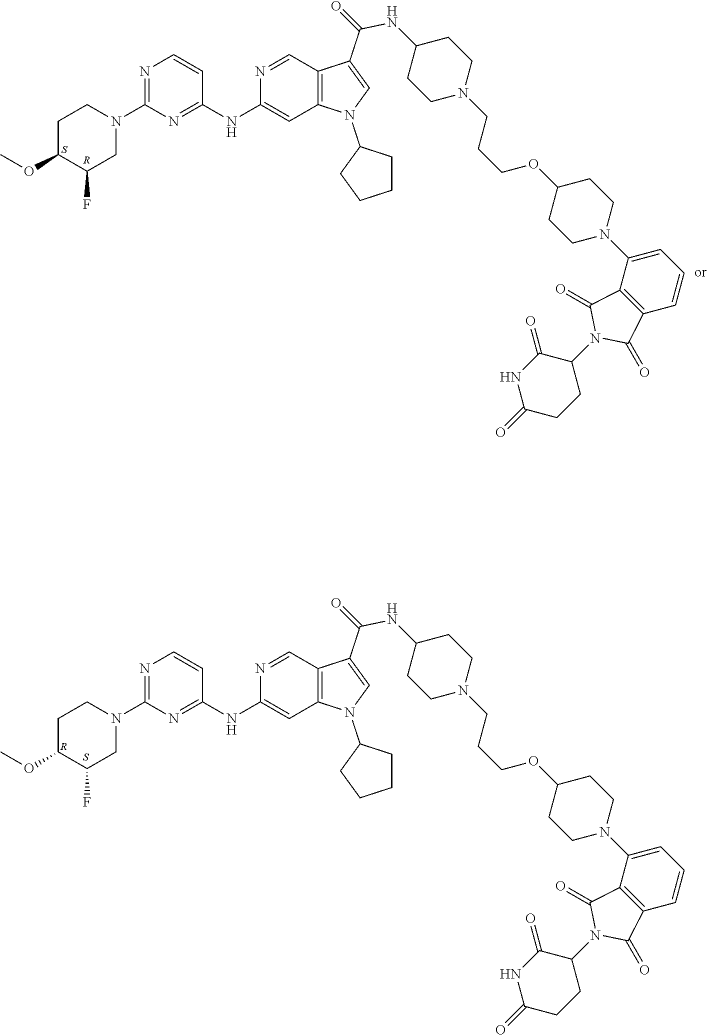

The title compound was obtained as a yellow solid, MS: m/e=934.9 (M-W), using chemistry similar to that described in Example 2, step 3 starting from 1-cyclopentyl-6-[[2-[(3R,4 S)-3-fluoro-4-methoxy-1-piperidyl]pyrimidin-4-yl]amino]-N-(4-piperidyl)pyrrolo[3,2-c]pyridine-3-carboxamide hydrochloride or 1-cyclopentyl-6-[[2-[(3S,4R)-3-fluoro-4-methoxy-1-piperidyl]pyrimidin-4-yl]amino]-N-(4-piperidyl)pyrrolo[3,2-c]pyridine-3-carboxamide hydrochloride (Example 10, step 1) and 4-[4-(3-bromoprop oxy)-1-piperidyl]-2-[(3RS)-2,6-dioxo-3-piperidyl]isoindoline-1,3-dione (Example 17, step 1).

### Example 19

### N-[1-[4-[1-[2-[(3RS)-2,6-Dioxo-3-piperidyl]-1,3-dioxo-isoindolin-5-yl]-4-piperidyl]butyl]-4-piperidyl]-6-[[2-[(3R,4S)(3S,4R)-3-fluoro-4-methoxy-1-piperidyl]pyrimidin-4-yl]amino]-1-isopropyl-pyrrolo[3,2-c]pyridine-3-carboxamide

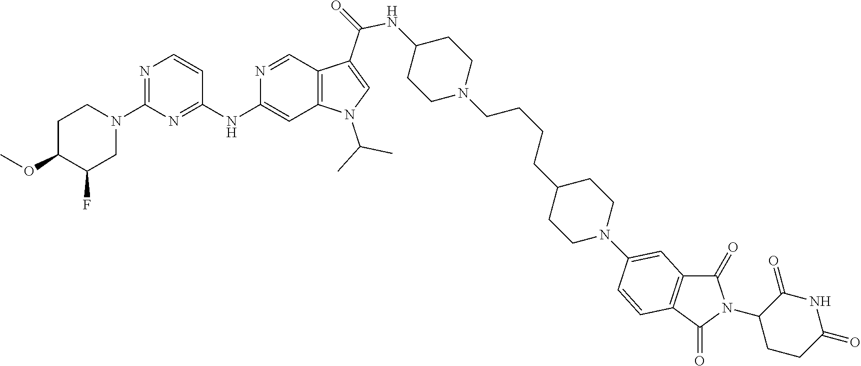 [SMILES: CO[C@H]1CCN(c2nccc(Nc3cc4c(cn3)c(C(=O)NC3CCN(CCCCC5CCN(c6ccc7c(c6)C(=O)N(C6CCC(=O)NC6=O)C7=O)CC5)CC3)cn4C(C)C)n2)C[C@H]1F]

### Step 1: tert-Butyl 4-(4-(4-(6-((2-((3R,4S)(3 S,4R)-3-fluoro-4-methoxypiperidin-1-yl)pyrimidin-4-yl)amino)-1-isopropyl-1H-pyrrolo[3,2-c]pyridine-3-carboxamido)piperidin-1-yl)butyl)piperidine-1-carboxylate

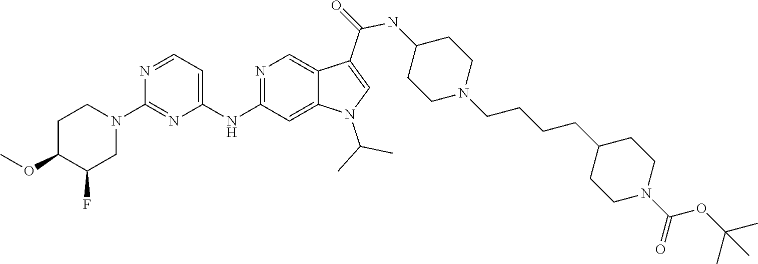 [SMILES: CO[C@H]1CCN(c2nccc(Nc3cc4c(cn3)c(C(=O)NC3CCN(CCCCC5CCN(C(=O)OC(C)(C)C)CC5)CC3)cn4C(C)C)n2)C[C@H]1F]

The title compound was obtained as a yellow foam, MS: m/e=750.8 (M+H⁺), using chemistry similar to that described in Example 12, step 2 starting from 6-((2-((3R,4S)(3S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride (Example 5, step 2) and 4-(1-boc-4-piperidyl)-1-butanol (CAS 142355-83-7).

### Step 2: N-[1-[4-[1-[2-[(3RS)-2,6-Dioxo-3-piperidyl]-1,3-dioxo-isoindolin-5-yl]-4-piperidyl]butyl]-4-piperidyl]-6-[[2-[(3R,4S)(3 S,4R)-3-fluoro-4-methoxy-l-piperidyl]pyrimidin-4-yl]amino]-1-isopropyl-pyrrolo[3,2-c]pyridine-3-carboxamide

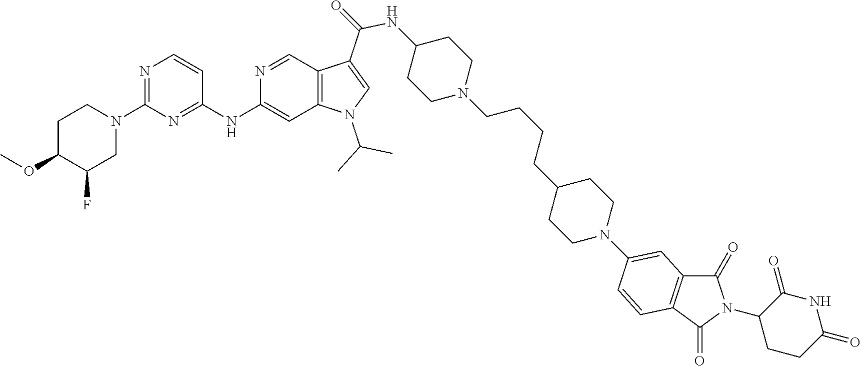 [SMILES: CO[C@H]1CCN(c2nccc(Nc3cc4c(cn3)c(C(=O)NC3CCN(CCCCC5CCN(c6ccc7c(c6)C(=O)N(C6CCC(=O)NC6=O)C7=O)CC5)CC3)cn4C(C)C)n2)C[C@H]1F]

tert-Butyl 4-[4-[4-[[6-[[2-[(3R,4 S)(3 S,4R)-3-fluoro-4-methoxy-1-piperidyl]pyrimidin-4-yl]amino]-1-isopropyl-pyrrolo[3,2-c]pyridine-3-carbonyl]amino]-1-piperidyl]butyl]piperidine-1-carboxylate (Example 19, step 1) (40 mg, 53.34 μmol) was dissolved in DCM (0.5 mL) and TFA (0.5 mL) was added. The mixture was stirred at room temperature for 30 minutes and concentrated. The residue was dissolved in DMAc (0.5 mL) and 2-[(3RS)-2,6-dioxo-3-piperidyl]-5-fluoro-isoindoline-1,3-dione (CAS 835616-61-0) (22.10 mg, 80.00 μmol, 1.5 equiv.) was added, followed by DIEA (92.90 μL, 533.36 μmol, 10 equiv.). The reaction was sealed and heated at 90° C. overnight. The mixture was purified by flash column chromatography on silica gel (0-20% 1.75 M ammonia in MeOH in DCM) to give N-[1-[4-[1-[2-[(3RS)-2,6-dioxo-3-piperidyl]-1,3-dioxo-isoindolin-5-yl]-4-piperidyl]butyl]-4-piperidyl]-6-[[2-[(3R,4S)(3 S,4R)-3-fluoro-4-methoxy-1-piperidyl]pyrimidin-4-yl]amino]-1-isopropyl-pyrrolo[3,2-c]pyridine-3-carboxamide (20 mg, 41% yield) as a yellow solid. MS: m/e=906.8 (M+H⁺).

### Example 20

### N-[1-[3-[4-[[2-[(3RS)-2,6-Dioxo-3-piperidyl]-1,3-dioxo-isoindolin-4-yl]amino]cyclohexyl]propyl]-4-piperidyl]-6-[[2-[(3R,4S)-3-fluoro-4-methoxy-l-piperidyl]pyrimidin-4-yl]amino]-1-isopropyl-pyrrolo[3,2-c]pyridine-3-carboxamide, or

### N-[1-[3-[4-[[2-[(3RS)-2,6-dioxo-3-piperidyl]-1,3-dioxo-isoindolin-4-yl]amino]cyclohexyl]propyl]-4-piperidyl]-6-[[2-[(3S,4R)-3-fluoro-4-methoxy-l-piperidyl]pyrimidin-4-yl]amino]-1-isopropyl-pyrrolo[3,2-c]pyridine-3-carboxamide

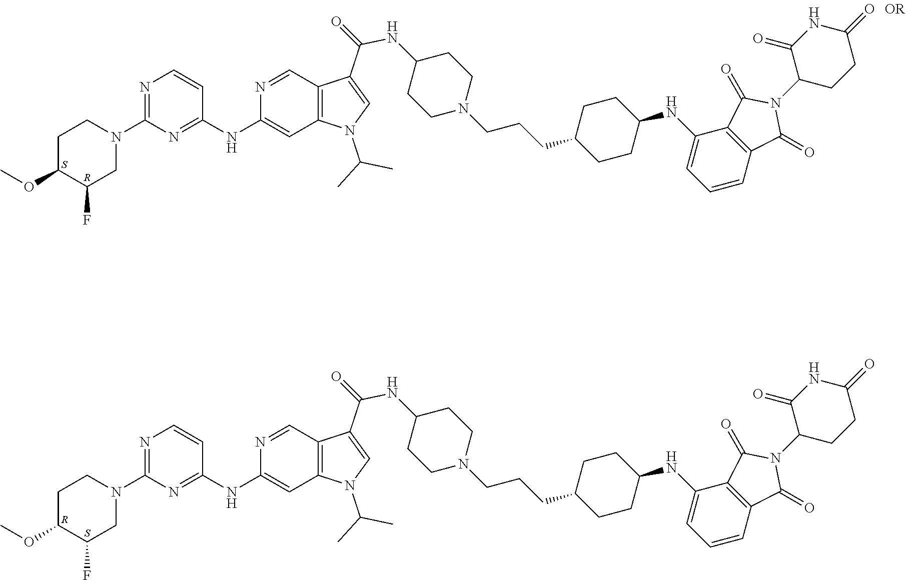

### Step 1: tert-Butyl (trans-4-(3-hydroxypropyl)cyclohexyl)carbamate

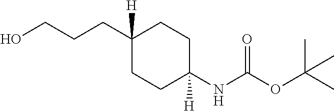 [SMILES: CC(C)(C)OC(=O)N[C@H]1CC[C@H](CCCO)CC1]

trans-3-[4-(Boc-amino)cyclohexyl]propanoic acid (CAS 204245-65-8) (0.500 g, 1.79 mmol) was dissolved in 7.0 ml of THF and cooled to 0-5° C. Borane-tetrahydrofuran complex (1M solution in THF) (2.7 ml, 2.7 mmol, 1.51 equiv.) was added dropwise at 0-5° C. and was stirred at 0-5° C. for 3 hours. The reaction mixture was quenched with 5 ml of 2M NaOH, stirred at 0-5° C. for 1 hour and then extracted with ethyl acetate and water. The aqueous layer was backextracted with ethyl acetate. The organic layers were washed with water and brine. The organic layers were combined, dried over sodium sulfate, filtered and evaporated to dryness. The crude product was adsorbed on Isolute® and purified by flash chromatography on a silica gel column eluting with an ethyl acetate:heptane 0:100 to 50:50 gradient to obtain the desired tert-butyl (trans-4-(3-hydroxypropyl)cyclohexyl)carbamate (386 mg, 84% yield) as an off-white solid, MS: m/e=515.5 (2M+H⁺).

### Step 2: tert-Butyl (trans-4-(3-bromopropyl)cyclohexyl)carbamate

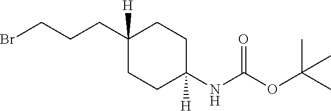 [SMILES: CC(C)(C)OC(=O)N[C@H]1CC[C@H](CCCBr)CC1]

tert-Butyl (trans-4-(3-hydroxypropyl)cyclohexyl)carbamate (Example 20, step 1) (0.180 g, 0.699 mmol) was dissolved in 10 ml of dichloromethane. Carbon tetrabromide (278 mg, 0.839 mmol, 1.2 equiv.) was added followed by triphenylphosphine (220 mg, 0.839 mmol, 1.2 equiv.) and the reaction mixture was stirred at room temperature for 5 hours. The reaction mixture was adsorbed on Isolute® and purified by flash chromatography on a silica gel column eluting with an ethyl acetate:heptane 0:100 to 10:90 gradient to obtain the desired tert-butyl (trans-4-(3-bromopropyl)cyclohexyl)carbamate (194 mg, 87% yield) as an off-white solid, MS: m/e=264.0/266.0 (M-tBu+H⁺).

### Step 3: tert-Butyl (trans-4-(3-(4-(6-((2-((3R,4S)-3-fluoro-4-methoxypiperidin-1-yl)pyrimidin-4-yl)amino)-1-isopropyl-1H-pyrrolo[3,2-c]pyridine-3-carboxamido)piperidin-l-yl)propyl)cyclohexyl)carbamate or tert-butyl (trans-4-(3-(4-(6-((2-((3 S,4R)-3-fluoro-4-methoxypiperidin-1-yl)pyrimidin-4-yl)amino)-1-isopropyl-1H-pyrrolo[3,2-c]pyridine-3-carboxamido)piperidin-1-yl)propyl)cyclohexyl)carbamate

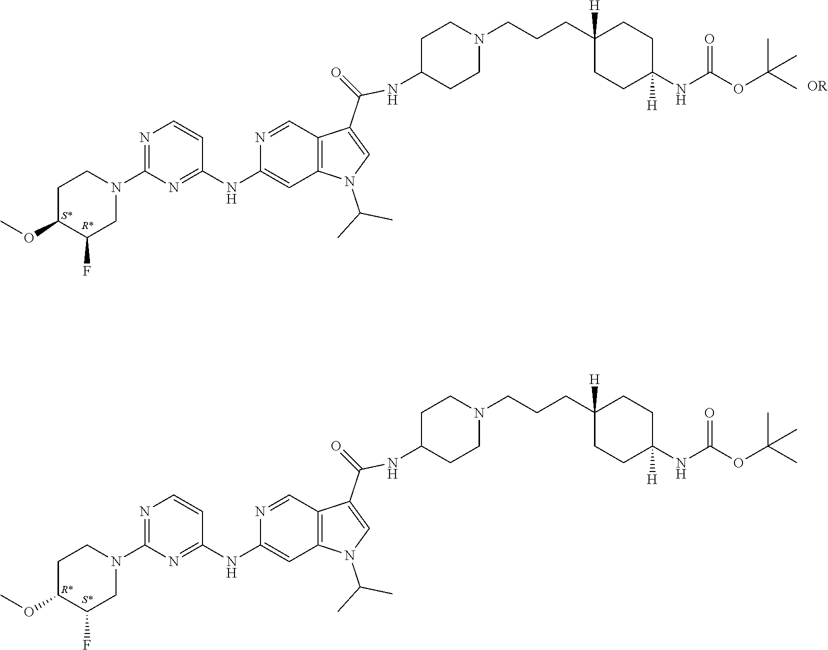

A screw-capped vial was charged with 6-((2-((3S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride or 6-((2-((3R,4S)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide hydrochloride (Example 8, step 2) (0.095 g, 0.167 mmol), tert-butyl (trans-4-(3-bromopropyl)cyclohexyl)carbamate (Example 20, step 2) (70 mg, 0.219 mmol, 1.31 equiv.), 1.6 ml of DMF and N,N-diisopropylethylamine (118 mg, 0.16 ml, 0.916 mmol, 5.47 equiv.). The vial was flushed with argon and stirred at 60° C. overnight. The reaction mixture was cooled to room temperature and then extracted with dichloromethane and water. The organic layer was washed with water. The aqueous layers were back-extracted twice with dichloromethane. The organic layers were combined, dried over sodium sulfate, filtered and evaporated to dryness. The crude product was adsorbed on Isolute® and purified by flash chromatography on a silica gel column eluting with dichloromethane:methanol 100:0 to 90:10 gradient to obtain the desired

tert-butyl (trans-4-(3-(4-(6-((2-((3R,4S)-3-fluoro-4-methoxypiperidin-1-yl)pyrimidin-4-yl)amino)-1-isopropyl-1H-pyrrolo[3,2-c]pyridine-3-carboxamido)piperidin-1-yl)propyl)cyclohexyl)carbamate or tert-butyl (trans-4-(3-(4-(6-((2-((3 S,4R)-3-fluoro-4-methoxypiperidin-1-yl)pyrimidin-4-yl)amino)-1-isopropyl-1H-pyrrolo[3,2-c]pyridine-3-carboxamido)piperidin-1-yl)propyl)cyclohexyl)carbamate (106 mg, 84% yield) as an off-white foam, MS: m/e=750.6 (M+H⁺).

### Step 4: N-(1-(3-(trans-4-Aminocyclohexyl)propyl)piperidin-4-yl)-6-((2-((3R,4S)-3-fluoro-4-methoxypiperidin-1-yl)pyrimidin-4-yl)amino)-1-isopropyl-1H-pyrrolo[3,2-c]pyridine-3-carboxamide or N-(1-(3-(trans-4-aminocyclohexyl)propyl)piperidin-4-yl)-6-((2-((3R,4S)-3-fluoro-4-methoxypiperidin-1-yl)pyrimidin-4-yl)amino)-1-isopropyl-1H-pyrrolo[3,2-c]pyridine-3-carboxamide

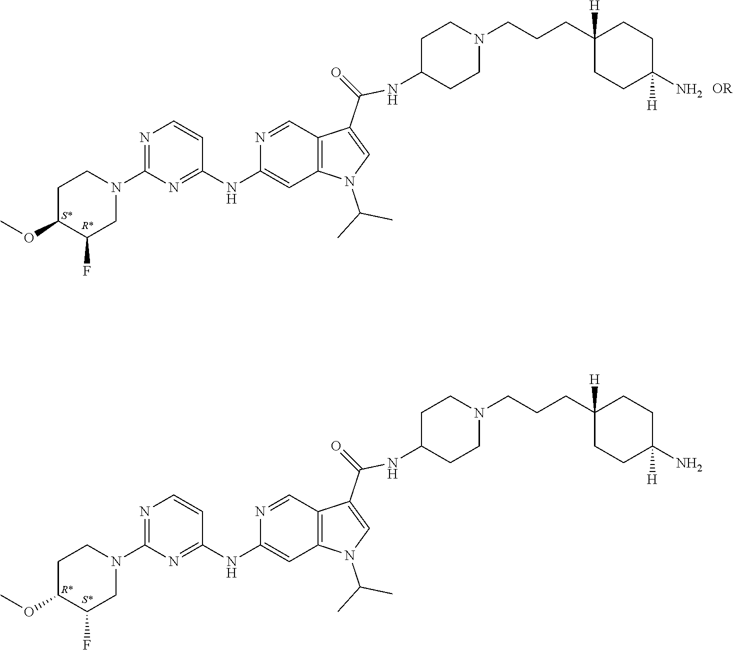 [SMILES: CO[C@H]1CCN(c2nccc(Nc3cc4c(cn3)c(C(=O)NC3CCN(CCC[C@H]5CC[C@H](N)CC5)CC3)cn4C(C)C)n2)C[C@H]1F ; CO[C@@H]1CCN(c2nccc(Nc3cc4c(cn3)c(C(=O)NC3CCN(CCC[C@H]5CC[C@H](N)CC5)CC3)cn4C(C)C)n2)C[C@@H]1F]

tert-Butyl (trans-4-(3-(4-(6-((2-((3R,4S)-3-fluoro-4-methoxypiperidin-1-yl)pyrimidin-4-yl)amino)-1-isopropyl-1H-pyrrolo[3,2-c]pyridine-3-carboxamido)piperidin-1-yl)propyl)cyclohexyl)carbamate or tert-butyl (trans-4-(3-(4-(6-((2-((3 S,4R)-3-fluoro-4-methoxypiperidin-1-yl)pyrimidin-4-yl)amino)-1-isopropyl-1H-pyrrolo[3,2-c]pyridine-3-carboxamido)piperidin-1-yl)propyl)cyclohexyl)carbamate (Example 20, step 3) (0.100 g, 0.133 mmol) was dissolved in 0.68 ml of dichloromethane and 0.34 ml of methanol. HCl (4N in dioxane) (0.34 ml, 1.36 mmol, 10.2 equiv.) was added dropwise. The reaction mixture was stirred at room temperature for 2 hours. The reaction mixture was evaporated to dryness. The residue was extracted with a mixture of dichloromethane/methanol (9:1) and saturated NaHCO₃-solution. The aqueous layer was backextracted twice with a mixture of dichloromethane/methanol (9:1). The organic layers were combined, dried over sodium sulfate, filtered and evaporated to dryness to afford N-(1-(3-(trans-4-aminocyclohexyl)propyl)piperidin-4-yl)-6-((2-((3R,4S)-3-fluoro-4-methoxypiperidin-1-yl)pyrimidin-4-yl)amino)-1-isopropyl-1H-pyrrolo[3,2-c]pyridine-3-carboxamide or N-(1-(3-(trans-4-aminocyclohexyl)propyl)piperidin-4-yl)-6-((2-((3R,4S)-3-fluoro-4-methoxypiperidin-1-yl)pyrimidin-4-yl)amino)-1-isopropyl-1H-pyrrolo[3,2-c]pyridine-3-carboxamide (86 mg, 94% yield) as a light yellow solid, MS: m/e=650.8 (M+H⁺).

### Step 5: N-(1-(3-(trans-4-((2-((3RS)-2,6-Dioxopiperidin-3-yl)-1,3-dioxoisoindolin-4-yl)amino)cyclohexyl)propyl)piperidin-4-yl)-6-((2-((3R,4S)-3-fluoro-4-methoxypiperidin-l-yl)pyrimidin-4-yl)amino)-1-isopropyl-1H-pyrrolo[3,2-c]pyridine-3-carboxamide

### Or

### N-(1-(3-(trans-4-((2-((3RS)-2,6-dioxopiperidin-3-yl)-1,3-dioxoisoindolin-4-yl)amino)cyclohexyl)propyl)piperidin-4-yl)-6-((2-((3 S,4R)-3-fluoro-4-methoxypiperidin-1-yl)pyrimidin-4-yl)amino)-1-isopropyl-1H-pyrrolo[3,2-c]pyridine-3-carboxamide

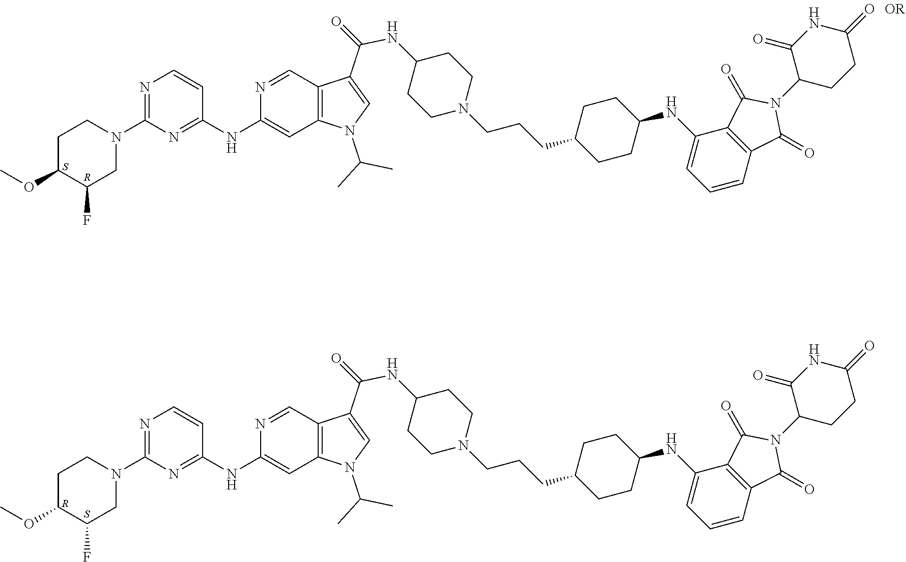

The title compound was obtained as a yellow foam, MS: m/e=907.0 (M+H⁺), using chemistry similar to that described in Example 1, step 7 starting from N-(1-(3-(trans-4-aminocyclohexyl)propyl)piperidin-4-yl)-6-((2-((3R,4S)-3-fluoro-4-methoxypiperidin-1-yl)pyrimidin-4-yl)amino)-1-isopropyl-1H-pyrrolo[3,2-c]pyridine-3-carboxamide or N-(1-(3-(trans-4-aminocyclohexyl)propyl)piperidin-4-yl)-6-((2-((3R,4S)-3-fluoro-4-methoxypiperidin-1-yl)pyrimidin-4-yl)amino)-1-isopropyl-1H-pyrrolo[3,2-c]pyridine-3-carboxamide (Example 20, step 4) and 2-[(3RS)-2,6-dioxo-3-piperidyl]-4-fluoro-isoindoline-1,3-dione (CAS 835616-60-9) by using dioxane/DMA (4:1) instead of DMSO as solvent.

### Example 21

### N-[1-[6-[[2-[(3RS)-2,6-Dioxo-3-piperidyl]-1,3-dioxo-isoindolin-4-yl]amino]hexyl]-4-piperidyl]-6-[[2-[(3S,4R)-3-fluoro-4-methoxy-1-piperidyl]pyrimidin-4-yl]amino]-1-isopropyl-pyrazolo[4,3-c]pyridine-3-carboxamide, or

### N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrazolo[4,3-c]pyridine-3-carboxamide

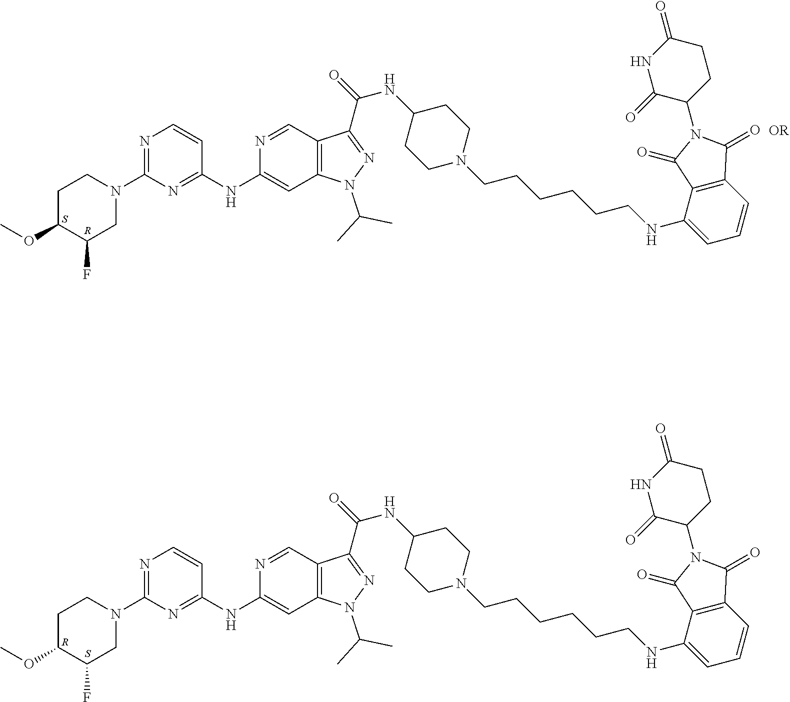

### Step 1: 6-[[2-[(3R,4S)-3-Fluoro-4-methoxy-1-piperidyl]pyrimidin-4-yl]amino]-1-isopropyl-N-(4-piperidyl)pyrazolo[4,3-c]pyridine-3-carboxamide or 6-[[2-[(3S,4R)-3-fluoro-4-methoxy-1-piperidyl]pyrimidin-4-yl]amino]-1-isopropyl-N-(4-piperidyl)pyrazolo[4,3-c]pyridine-3-carboxamide

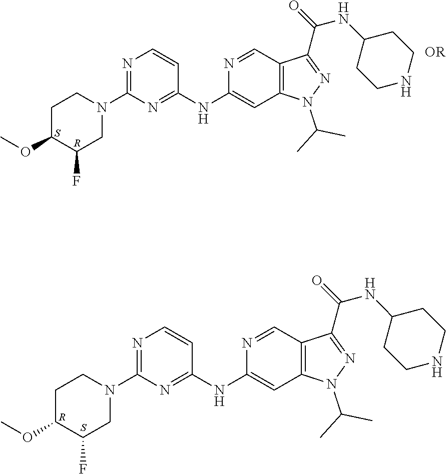

The title compound was obtained as a light yellow solid, MS: m/e=512.4 (M+H⁺), using chemistry similar to that described in Example 1, step 3 to step 6 starting from methyl 6-chloro-1-isopropyl-pyrazolo[4,3-c]pyridine-3-carboxylate (CAS 1643499-78-8) and 2-((3R,4S)-3-fluoro-4-methoxypiperidin-1-yl)pyrimidin-4-amine or 2-((3 S,4R)-3-fluoro-4-methoxypiperidin-1-yl)pyrimidin-4-amine (Example 7, step 1).

### Step 2: N-[1-[6-[[2-[(3RS)-2,6-Dioxo-3-piperidyl]-1,3-dioxo-isoindolin-4-yl]amino]hexyl]-4-piperidyl]-6-[[2-[(3 S,4R)-3-fluoro-4-methoxy-1-piperidyl]pyrimidin-4-yl]amino]-1-isopropyl-pyrazolo[4,3-c]pyridine-3-carboxamide or N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrazolo[4,3-c]pyridine-3-carboxamide

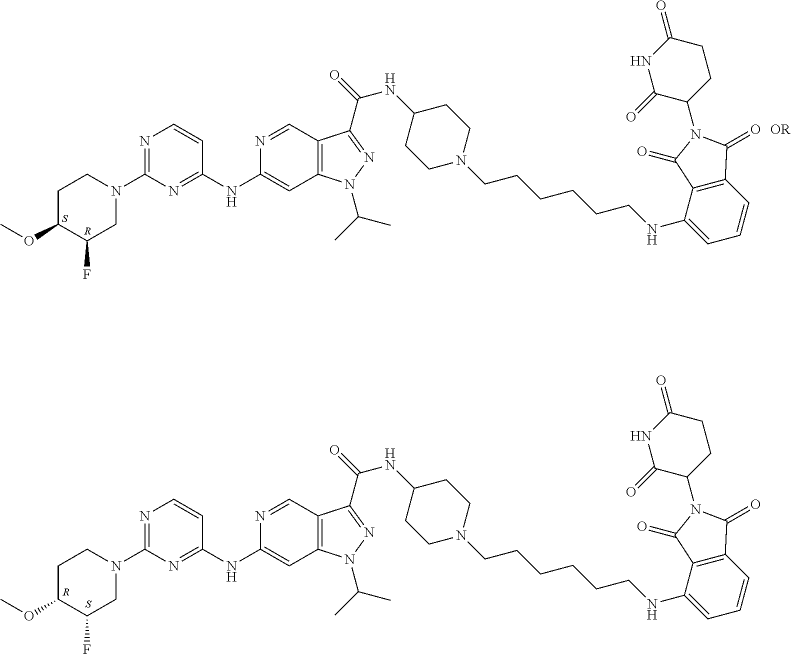

The title compound was obtained as a yellow foam, MS: m/e=867.6 (M+H⁺), using chemistry similar to that described in Example 2, step 3 starting from 6-[[2-[(3R,48)-3-fluoro-4-methoxy-1-piperidyl]pyrimidin-4-yl]amino]-1-isopropyl-N-(4-piperidyl)pyrazolo[4,3-c]pyridine-3-carboxamide or 6-[[2-[(3 S,4R)-3-fluoro-4-methoxy-1-piperidyl]pyrimidin-4-yl]amino]-1-isopropyl-N-(4-piperidyl)pyrazolo[4,3-c]pyridine-3-carboxamide (Example 21, step 1) and 4-(6-bromohexylamino)-2-[(3RS)-2,6-dioxo-3-piperidyl]isoindoline-1,3-dione (Example 2, step 2).

### Example 22

### N-[1-[3-[1-[2-[(3RS)-2,6-Dioxo-3-piperidyl]-1,3-dioxo-isoindolin-4-yl]azetidin-3-yl]oxypropyl]-4-piperidyl]-6-[[2-[(3R,4S)-3-fluoro-4-methoxy-l-piperidyl]pyrimidin-4-yl]amino]-1-isopropyl-pyrrolo[3,2-c]pyridine-3-carboxamide, or

### N-[1-[3-[1-[2-[(3RS)-2,6-dioxo-3-piperidyl]-1,3-dioxo-isoindolin-4-yl]azetidin-3-yl]oxypropyl]-4-piperidyl]-6-[[2-[(3S,4R)-3-fluoro-4-methoxy-l-piperidyl]pyrimidin-4-yl]amino]-1-isopropyl-pyrrolo[3,2-c]pyridine-3-carboxamide

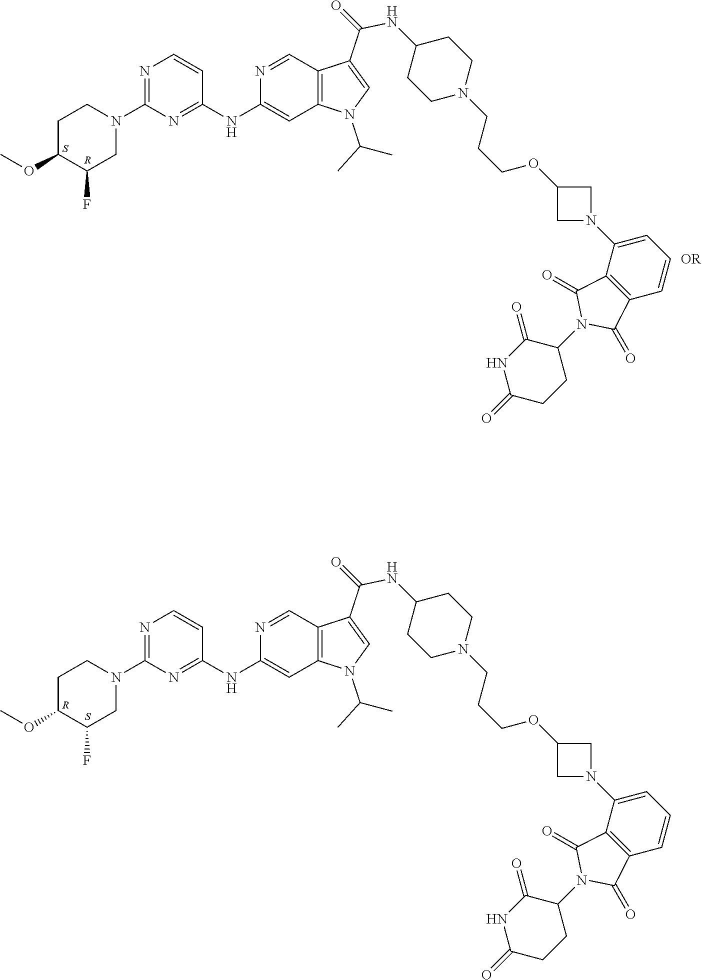

### Step 1: tert-Butyl 3-allyloxyazetidine-1-carboxylate

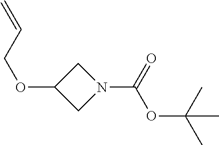

tert-Butyl 3-hydroxyazetidine-1-carboxylate (CAS 141699-55-0) (800 mg, 4.62 mmol) was dissolved in 9 ml of DMF and cooled to 0-5° C. Sodium hydride (60% in mineral oil) (277 mg, 6.93 mmol, 1.5 equiv.) was added carefully in portions at 0-5° C. and the reaction mixture was stirred at 0-5° C. for 10 minutes. Allyl bromide (1.12 g, 0.80 ml, 9.24 mmol, 2 equiv.) was added dropwise at 0-5° C. and the reaction mixture was stirred at room temperature for 3 hours. The reaction mixture was extracted with MTBE and water. The aqueous layer was back-extracted with MTBE. The organic layers were washed three times with water and once with brine. The organic layers were combined, dried over sodium sulfate, filtered and evaporated to dryness. The crude product was adsorbed on Isolute® and purified by flash chromatography on a silica gel column eluting with an ethyl acetate:heptane 0:100 to 20:80 gradient to obtain the desired tert-butyl 3-allyloxyazetidine-1-carboxylate (880 mg, 89% yield) as a colorless oil, MS: m/e=158.1 (M-tBu+H⁺).

### Step 2: tert-Butyl 3-(3-hydroxypropoxy)azetidine-1-carboxylate

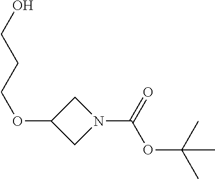 [SMILES: CC(C)(C)OC(=O)N1CC(OCCCO)C1]

tert-Butyl 3-allyloxyazetidine-1-carboxylate (Example 22, step 1) (872 mg, 4.09 mmol) was dissolved in 14 ml of THF and cooled to 0-5° C. A solution of 9-BBN (0.5 M in THF) (20.0 ml, 10 mmol, 2.45 equiv.) was added dropwise at 0-5° C. The reaction mixture was stirred at room temperature for 16 hours. The reaction mixture was cooled to 0-5° C. Hydrogen peroxide, 35 wt. % solution in water (2.55 g, 2.3 ml, 26.3 mmol, 6.43 equiv.) was added dropwise at 0-5° C. followed by 4M aq. sodium hydroxide-solution (4.6 ml, 18.4 mmol, 4.5 equiv.). After the addition was complete, the reaction mixture was stirred at room temperature for 1.5 hours. The reaction mixture was extracted with MTBE and saturated NaHCO₃-solution. The aqueous layer was backextracted twice MTBE. The organic layers were combined, dried over sodium sulfate, filtered and evaporated to dryness. The crude product was purified by flash chromatography on a silica gel column eluting with an ethyl acetate:heptane 0:100 to 60:40 gradient to obtain the desired tert-butyl 3-(3-hydroxypropoxy)azetidine-1-carboxylate (557 mg, 59% yield) as a colorless oil, MS: m/e=176.1 (M-tBu+H⁺).

### Step 3: tert-Butyl 3-(3-bromopropoxy)azetidine-1-carboxylate

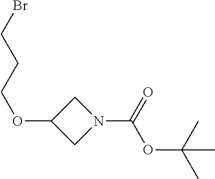 [SMILES: CC(C)(C)OC(=O)N1CC(OCCCBr)C1]

tert-Butyl 3-(3-hydroxypropoxy)azetidine-l-carboxylate (Example 22, step 2) (385 mg, 1.66 mmol) was dissolved in 24 ml of dichloromethane. Carbon tetrabromide (662 mg, 2 mmol, 1.2 equiv.) and triphenylphosphine (524 mg, 2 mmol, 1.2 equiv.) were added and the reaction mixture was stirred at room temperature for 5 hours. The reaction mixture was adsorbed on Isolute® and purified by flash chromatography on a silica gel column eluting with an ethyl acetate:heptane 0:100 to 20:80 gradient to obtain the desired tert-butyl 3-(3-bromopropoxy)azetidine-1-carboxylate (375 mg, 77% yield) as a colorless oil, MS: m/e=239.2/240.2 (M-tBu+H⁺).

### Step 4: tert-Butyl 3-[3-[4-[[1-isopropyl-6-[[2-[rac-(3R,4S)-3-fluoro-4-methoxy-1-piperidyl]pyrimidin-4-yl]amino]pyrrolo[3,2-c]pyridine-3-carbonyl]amino]-1-piperidyl]propoxy]azetidine-1-carboxylate or tert-butyl 3-[3-[4-[[1-isopropyl-6-[[2-[rac-(3 S,4R)-3-fluoro-4-methoxy-1-piperidyl]pyrimidin-4-yl]amino]pyrrolo[3,2-c]pyridine-3-carbonyl]amino]-1-piperidyl]propoxy]azetidine-1-carboxylate

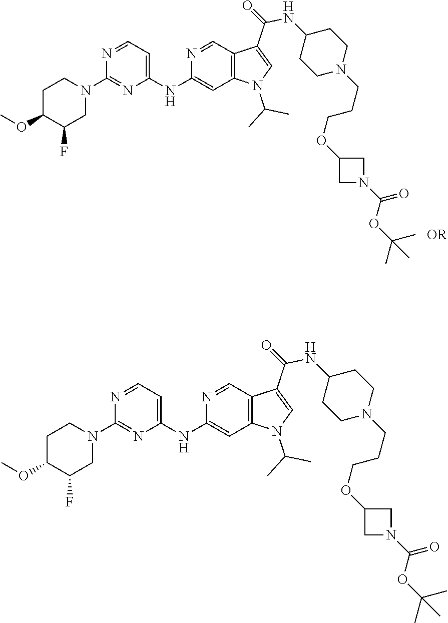

The title compound was obtained as a white foam, MS: m/e=724.8 (M+H⁺), using chemistry similar to that described in Example 20, step 3 starting from 6-((2-((3S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide or 6-((2-((3R,4S)-3-fluoro-4-methoxypiperidin-1-yl)pyrimidin-4-yl)amino)-1-isopropyl-N-(piperidin-4-yl)-1H-pyrrolo[3,2-c]pyridine-3-carboxamide (Example 8, step 2) as a free base and tert-butyl 3-(3-bromopropoxy)azetidine-1-carboxylate (Example 22, step 3).

### Step 5: N-[1-[3-(Azetidin-3-yloxy)propyl]-4-piperidyl]-1-isopropyl-6-[[2-[rac-(3R,4S)-3-fluoro-4-methoxy-1-piperidyl]pyrimidin-4-yl]amino]pyrrolo[3,2-c]pyridine-3-carboxamide or N-[1-[3-(azetidin-3-yloxy)propyl]-4-piperidyl]-1-isopropyl-6-[[2-[rac-(3 S, 4R)-3-fluoro-4-methoxy-1-piperidyl]pyrimidin-4-yl]amino]pyrrolo[3,2-c]pyridine-3-carboxamide

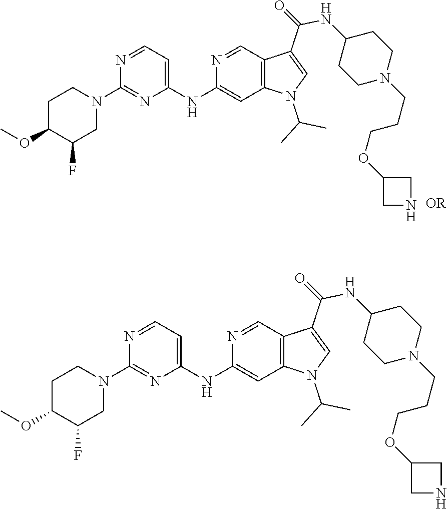 [SMILES: CO[C@H]1CCN(c2nccc(Nc3cc4c(cn3)c(C(=O)NC3CCN(CCCOC5CNC5)CC3)cn4C(C)C)n2)C[C@H]1F ; CO[C@@H]1CCN(c2nccc(Nc3cc4c(cn3)c(C(=O)NC3CCN(CCCOC5CNC5)CC3)cn4C(C)C)n2)C[C@@H]1F]

tert-Butyl 3-[3-[4-[[1-isopropyl-6-[[2-[rac-(3R,4S)-3-fluoro-4-methoxy-1-piperidyl]pyrimidin-4-yl]amino]pyrrolo[3,2-c]pyridine-3-carbonyl]amino]-1-piperidyl]propoxy]azetidine-1-carboxylate or tert-butyl 3-[3-[4-[[1-isopropyl-6-[[2-[rac-(3 S,4R)-3-fluoro-4-methoxy-1-piperidyl]pyrimidin-4-yl]amino]pyrrolo[3,2-c]pyridine-3-carbonyl]amino]-1-piperidyl]propoxy]azetidine-1-carboxylate (Example 22, step 4) (95 mg, 0.131 mmol) was dissolved in 1.3 ml of dichloromethane and cooled to 0-5° C. Trifluoroacetic acid (444 mg, 0.30 ml, 3.89 mmol, 29.7 equiv.) was added dropwise at 0-5° C. After the addition was complete, the ice bath was removed and the reaction mixture was stirred at room temperature for 30 minutes. The reaction mixture was evaporated to dryness. The residue was extracted with dichloromethane and saturated Na₂CO₃-solution. The aqueous layer was back-extracted twice with dichloromethane. The organic layers were combined, dried over sodium sulfate, filtered and evaporated to dryness to afford N-[1-[3-(azetidin-3-yloxy)propyl]-4-piperidyl]-1-isopropyl-6-[[2-[rac-(3R,4S)-3-fluoro-4-methoxy-1-piperidyl]pyrimidin-4-yl]amino]pyrrolo[3,2-c]pyridine-3-carboxamide or N-[1-[3-(azetidin-3-yloxy)propyl]-4-piperidyl]-1-isopropyl-6-[[2-[rac-(3R,4S)-3-fluoro-4-methoxy-l-piperidyl]pyrimidin-4-yl]amino]pyrrolo[3,2-c]pyridine-3-carboxamide (82 mg, 95% yield) as a white foam, MS: m/e=624.6 (M+H⁺).

### Step 6: N-[1-[3-[1-[2-[(3RS)-2,6-Dioxo-3-piperidyl]-1,3-dioxo-isoindolin-4-yl]azetidin-3-yl]oxypropyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide, or

### N-[1-[3-[1-[2-[(3RS)-2,6-dioxo-3-piperidyl]-1,3-dioxo-isoindolin-4-yl]azetidin-3-yl]oxypropyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide

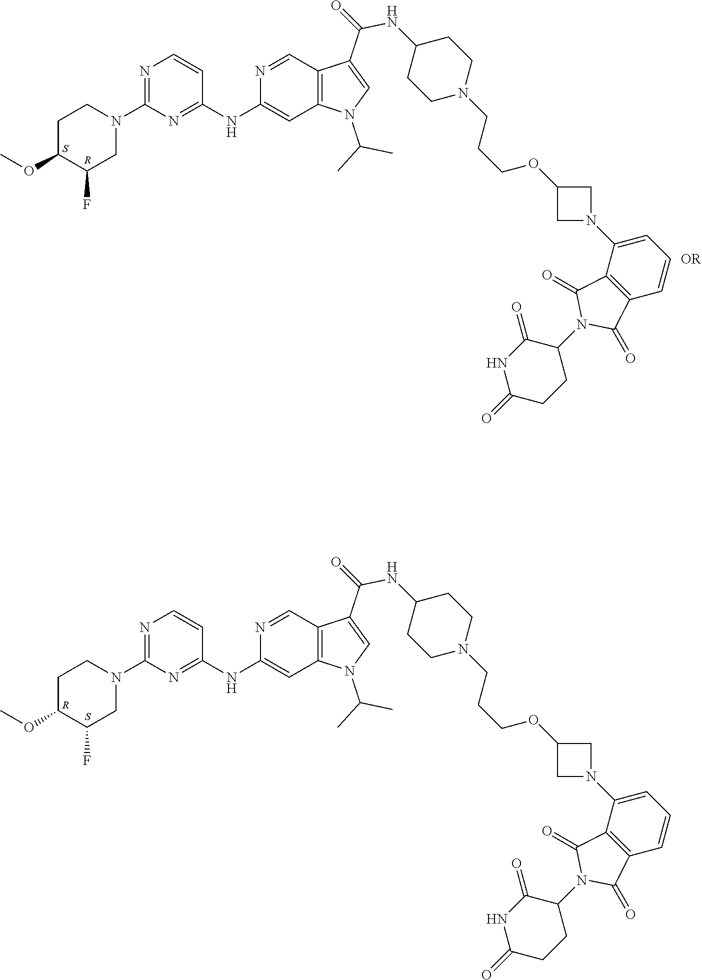

The title compound was obtained as a yellow solid, MS: m/e=880.8 (M+H⁺), using chemistry similar to that described in Example 1, step 7 starting from N-[1-[3-(azetidin-3-yloxy)propyl]-4-piperidyl]-1-isopropyl-6-[[2-[rac-(3R,4S)-3-fluoro-4-methoxy-1-piperidyl]pyrimidin-4-yl]amino]pyrrolo[3,2-c]pyridine-3-carboxamide or N-[1-[3-(azetidin-3-yloxy)propyl]-4-piperidyl]-1-isopropyl-6-[[2-[rac-(3R,4S)-3-fluoro-4-methoxy-1-piperidyl]pyrimidin-4-yl]amino]pyrrolo[3,2-c]pyridine-3-carboxamide (Example 22, step 5) and 2-[(3RS)-2,6-dioxo-3-piperidyl]-4-fluoro-isoindoline-1,3-dione (CAS 835616-60-9) and by using dioxane/DMA (5:1) solvent instead of DMSO.

### Example 23

### N-[1-[2-[1-[2-[(3RS)-2,6-Dioxo-3-piperidyl]-1,3-dioxo-isoindolin-4-yl]azetidin-3-yl]ethyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide

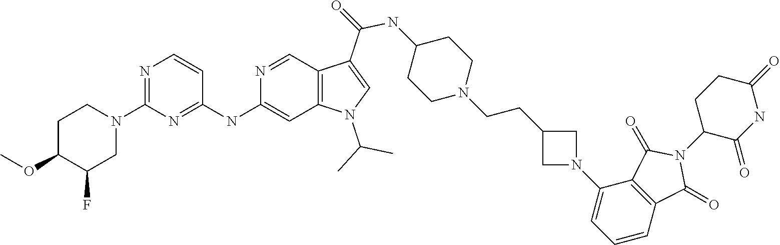

### Step 1: 2-[(3RS)-2,6-Dioxo-3-piperidyl]-4-[3-(2-hydroxyethyl)azetidin-1-yl]isoindoline-1,3-dione

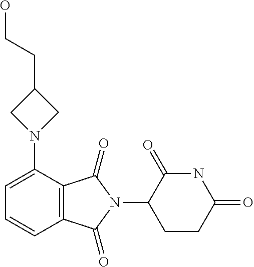 [SMILES: O=C1CCC(N2C(=O)c3cccc(N4CC(CCO)C4)c3C2=O)C(=O)N1]

The title compound was obtained as a yellow foam, MS: m/e=358.7 (M+H⁺), using chemistry similar to that described in Example 1, step 7 starting from 2-[(3RS)-2,6-dioxo-3-piperidyl]-4-fluoro-isoindoline-1,3-dione (CAS 835616-60-9) and 2-(azetidin-3-yl)ethanol (CAS 752956-75-5).

### Step 2: N-[1-[2-[1-[2-[(3RS)-2,6-Dioxo-3-piperidyl]-1,3-dioxo-isoindolin-4-yl]azetidin-3-yl]ethyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide

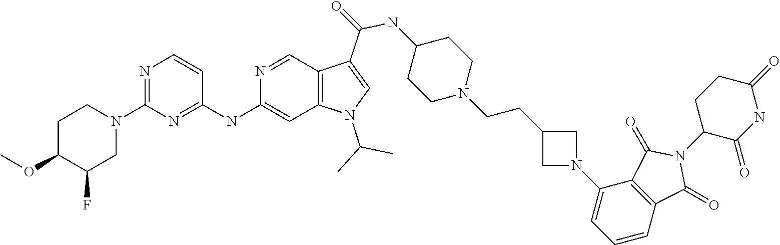

The title compound was obtained as a yellow solid, MS: m/e=850.4 (M+H+), using chemistry similar to that described in Example 12, step 2 starting from 6-((2-((3R,4S)(3S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carb oxamide hydrochloride (Example 5, step 2) and 2-[(312S)-2,6-dioxo-3-piperidyl]-4-[3-(2-hydroxyethyl)azetidin-l-yl]isoindoline-1,3-dione (Example 23, step 1).

### Example 24

### N-[1-[3-[1-[2-[(3RS)-2,6-Dioxo-3-piperidyl]-1,3-dioxo-isoindolin-4-yl]azetidin-3-yl]propyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide

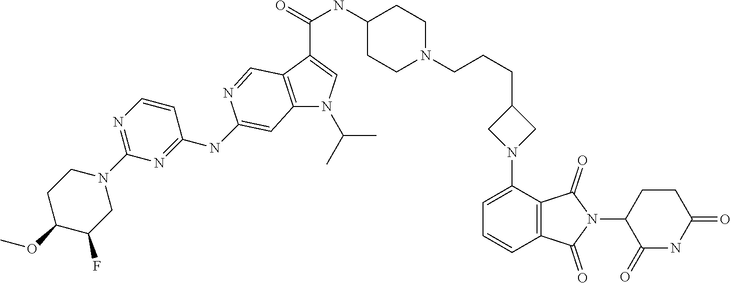

### Step 1: 2-[(3RS)-2,6-Dioxo-3-piperidyl]-4-[3-(3-hydroxypropyl)azetidin-1-yl]isoindoline-1,3-dione

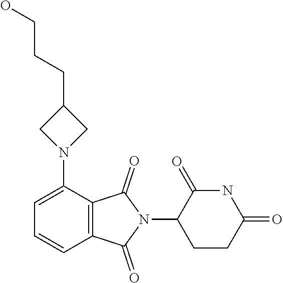

The title compound was obtained as a yellow foam, MS: m/e=372.7 (M+H⁺), using chemistry similar to that described in Example 1, step 7 starting from 2-[(3RS)-2,6-dioxo-3-piperidyl]-4-fluoro-isoindoline-1,3-dione (CAS 835616-60-9) and 3-(azetidin-3-yl)propan-1-ol (CAS 1379377-40-8).

### Step 2: N-(1-(3-(1-(2-(2,6-Dioxopiperidin-3-yl)-1,3-dioxoisoindolin-4-yl)azetidin-3-yl)propyl)piperidin-4-yl)-6-((2-((3R,4S)(3 S,4R)-3-fluoro-4-methoxypiperidin-1-yl)pyrimidin-4-yl)amino)-1-isopropyl-1H-pyrrolo[3,2-c]pyridine-3-carboxamide

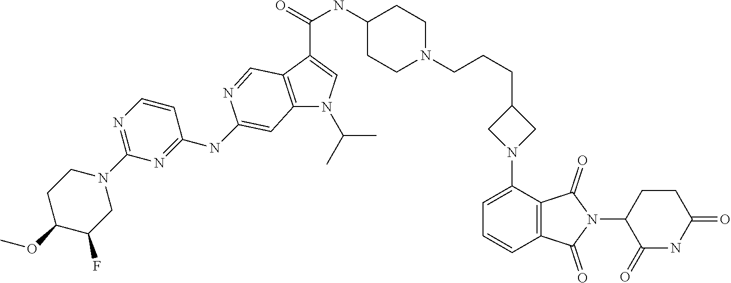

The title compound was obtained as a yellow solid, MS: m/e=864.7 (M+H+), using chemistry similar to that described in Example 12, step 2 starting from 6-((2-((3R,4S)(3S,4R)-3-fluoro-4-methoxypiperidin-1-yl)pyrimidin-4-yl)amino)-1-isopropyl-N-(piperidin-4-yl)-1H-pyrrolo[3,2-c]pyridine-3-carb oxamide hydrochloride (Example 5, step 2) and 2-[(3RS)-2,6-dioxo-3-piperidyl]-4-[3-(3-hydroxypropyl)azetidin-l-yl]isoindoline-1,3-dione (Example 24, step 1).

We claim:

1. A compound of formula I, or a pharmaceutically acceptable salt thereof, 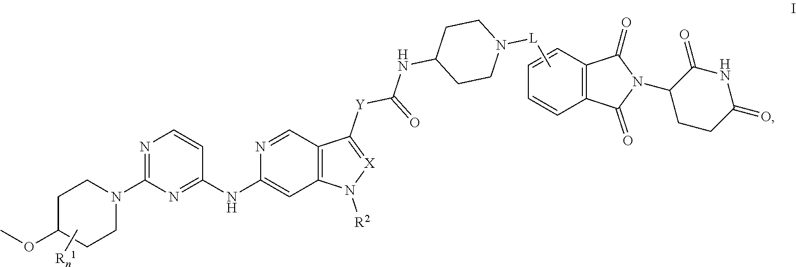
wherein
L is selected from the group consisting of:
i) —C(═O)—(CH₂)₂₋₁₀—NH—;
ii) —(CH₂)₂₋₁₀—NH—;
iii) —(CH₂)₂₋₁₀-heterocyclyl-;
iv) —C(═O)-heterocyclyl-(CH₂)₂₋₁₀—NH—;
v) —(CH₂)₂₋₁₀—O-heterocyclyl-; and
vi) —(CH₂)₂₋₁₀—C₃₋₆cycloalkyl-;
X is N or CH;
Y is absent or heterocyclyl;
n is 0, 1, or 2;
R¹ is each individually halogen;
R² is selected from the group consisting of
i.) C₁₋₆alkyl; and
ii.) C₃₋₆cycloalkyl.

2. The compound of claim 1, or pharmaceutically acceptable salts thereof, wherein L is selected from the group consisting of:
i) —C(═O)—(CH₂)₅—NH—;
ii) —(CH₂)₆—NH—;
iii) —(CH₂)₅—NH—;
iv) —(CH₂)₄—NH—;
v) —(CH₂)₄-piperidyl-;
vi) —(CH₂)₃-piperidyl-;
vii) —(CH₂)₂-piperidyl-;
viii) —(CH₂)₃-azetidinyl-;
ix) —(CH₂)₂-azetidinyl-;
x) —C(═O)-piperazinyl-(CH₂)₄—NH—;
xi) —(CH₂)₃—O-piperidyl-;
xii) —(CH₂)₃—O-azetidinyl-; and
xiii) —(CH₂)₃-cyclohexyl.

3. The compound of claim 1, or pharmaceutically acceptable salts thereof, wherein L is —(CH₂)₂₋₁₀—NH—.

4. The compound of claim 1, or pharmaceutically acceptable salts thereof, wherein L is —(CH₂)₆—NH—, —(CH₂)₅—NH—, or —(CH₂)₄—NH—.

5. The compound of claim 1, or pharmaceutically acceptable salts thereof, wherein Y is pyrrolidinyl.

6. The compound of claim 1, or pharmaceutically acceptable salts thereof, wherein Y is absent.

7. The compound of claim 1, or pharmaceutically acceptable salts thereof, wherein R¹ is F.

8. The compound of claim 1, or pharmaceutically acceptable salts thereof, wherein X is CH.

9. The compound of claim 1, or pharmaceutically acceptable salts thereof, wherein R² is C₁₋₆alkyl.

10. The compound of claim 1, or pharmaceutically acceptable salts thereof, wherein R² is iso-propyl.

11. The compound of claim 1, or pharmaceutically acceptable salts thereof, wherein R² is C₃₋₇cycloalkyl.

12. The compound of claim 1, or pharmaceutically acceptable salts thereof, wherein R² is cyclopentyl.

13. The compound of claim 1, or pharmaceutically acceptable salts thereof, selected from the group consisting of:
(3RS)—N-[1-[6-[[2-[(3RS)-2,6-dioxo-3-piperidyl]-1,3-dioxo-isoindolin-4-yl]amino]hexyl]-4-piperidyl]-1-[1-isopropyl-6-[[2-(4-methoxy-1-piperidyl)pyrimidin-4-yl]amino]pyrazolo[4,3-c]pyridin-3-yl]pyrrolidine-3-carboxamide,
1-Cyclopentyl-N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide,
1-cyclopentyl-N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide,
1-Cyclopentyl-N-[1-[3-[[1-[2-[(3 S)-2,6-dioxo-3-piperidyl]-1,3-dioxo-isoindolin-4-yl]-4-piperidyl]oxy]propyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide,
1-cyclopentyl-N-[1-[3-[[1-[2-[(3 S)-2,6-dioxo-3-piperidyl]-1,3-dioxo-isoindolin-4-yl]-4-piperidyl]oxy]propyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide,
1-Cyclopentyl-N-[1-[4-[4-[4-[2-[(3RS)-2,6-dioxo-3-piperidyl]-1,3-dioxo-isoindolin-4-yl]amino]butyl]piperazine-1-carbonyl]-4-piperidyl]-6-[[2-[(3R,4 S)-3-fluoro-4-methoxy-1-piperidyl]pyrimidin-4-yl]amino]pyrrolo[3,2-c]pyridine-3-carboxamide,
1-cyclopentyl-N-[1-[4-[4-[[2-[(3RS)-2,6-dioxo-3-piperidyl]-1,3-dioxo-isoindolin-4-yl]amino]butyl]piperazine-1-carbonyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide,
1-Cyclopentyl-N-[1-[5-[[2-[(3RS)-2,6-dioxo-3-piperidyl]-1,3-dioxo-isoindolin-4-yl]amino]pentyl]-4-piperidyl]-6-[[2-[(3R,4S)-3-fluoro-4-methoxy-1-piperidyl]pyrimidin-4-yl]amino]pyrrolo[3,2-c]pyridine-3-carboxamide,
1-cyclopentyl-N-[1-[5-[[2-[(3RS)-2,6-dioxo-3-piperidyl]-1,3-dioxo-isoindolin-4-yl]amino]pentyl]-4-piperidyl]-6-[[2-[(3 S,4R)-3-fluoro-4-methoxy-1-piperidyl]pyrimidin-4-yl]amino]pyrrolo[3,2-c]pyridine-3-carboxamide, and
1-Cyclopentyl-N-[1-[6-[[2-[(3RS)-2,6-dioxo-3-piperidyl]-1,3-dioxo-isoindolin-4-yl]amino]hexyl]-4-piperidyl]-6-[[2-(4-methoxy-1-piperidyl)pyrimidin-4-yl]amino]pyrrolo[3,2-c]pyridine-3-carboxamide.

14. The compound of claim 1, or pharmaceutically acceptable salts thereof, selected from the group consisting of:
1-cyclopentyl-N-[1-[6-[[2-[(3RS)-2,6-dioxo-3-piperidyl]-1,3-dioxo-isoindolin-4-yl]amino]hexyl]-4-piperidyl]-6-[[2-[(3R,4 S)-3-fluoro-4-methoxy-1-piperidyl]pyrimidin-4-yl]amino]pyrrolo[3,2-c]pyridine-3-carboxamide,
1-cyclopentyl-N-[1-[6-[[2-[(3RS)-2,6-dioxo-3-piperidyl]-1,3-dioxo-isoindolin-4-yl]amino]hexyl]-4-piperidyl]-6-[[2-[(3S,4R)-3-fluoro-4-methoxy-1-piperidyl]pyrimidin-4-yl]amino]pyrrolo[3,2-c]pyridine-3-carboxamide,
6-[[2-[(4RS)-3,3-Difluoro-4-methoxy-1-piperidyl]pyrimidin-4-yl]amino]-N-[1-[6-[[2-[(3RS)-2,6-dioxo-3-piperidyl]-1,3-dioxo-isoindolin-4-yl]amino]hexyl]-4-piperidyl]-1-isopropyl-pyrrolo[3,2-c]pyridine-3-carboxamide,
N-[1-[2-[1-[2-[(3RS)-2,6-Dioxo-3-piperidyl]-1,3-dioxo-isoindolin-5-yl]-4-piperidyl]ethyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[2-[1-[2-[(3RS)-2,6-dioxo-3-piperidyl]-1,3-dioxo-isoindolin-5-yl]-4-piperidyl]ethyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide,
N-[1-[2-[1-[2-[(3RS)-2,6-Dioxo-3-piperidyl]-1,3-dioxo-isoindolin-4-yl]azetidin-3-yl]ethyl]-4-piperidyl]-6-[[2-[(3R,4 S)-3-fluoro-4-methoxy-1-piperidyl]pyrimidin-4-yl]amino]-1-isopropyl-pyrrolo[3,2-c]pyridine-3-carboxamide,
N-[1-[3-[[1-[2-[(3RS)-2,6-Dioxo-3-piperidyl]-1,3-dioxo-isoindolin-4-yl]-4-piperidyl]oxy]propyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[3-[[1-[2-[(3RS)-2,6-dioxo-3-piperidyl]-1,3-dioxo-isoindolin-4-yl]-4-piperidyl]oxy]propyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide,
N-[1-[3-[1-[2-[(3RS)-2,6-Dioxo-3-piperidyl]-1,3-dioxo-isoindolin-4-yl]-4-piperidyl]propyl]-4-piperidyl]-6-[[2-[(3R,4S)(3S,4R)-3-fluoro-4-methoxy-1-piperidyl]pyrimidin-4-yl]amino]-1-isopropyl-pyrrolo[3,2-c]pyridine-3-carboxamide trifluoroacetate,
N-[1-[3-[1-[2-[(3RS)-2,6-Dioxo-3-piperidyl]-1,3-dioxo-isoindolin-4-yl]azetidin-3-yl]oxypropyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[3-[1-[2-[(3RS)-2,6-dioxo-3-piperidyl]-1,3-dioxo-isoindolin-4-yl]azetidin-3-yl]oxypropyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide, and
N-[1-[3-[1-[2-[(3RS)-2,6-Dioxo-3-piperidyl]-1,3-dioxo-isoindolin-4-yl]azetidin-3-yl]propyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide.

15. The compound of claim 1, or pharmaceutically acceptable salts thereof, selected from the group consisting of:
N-[1-[3-[4-[[2-[(3RS)-2,6-dioxo-3-piperidyl]-1,3-dioxo-isoindolin-4-yl]amino]cyclohexyl]propyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[3-[4-[[2-[(3RS)-2,6-dioxo-3-piperidyl]-1,3-dioxo-isoindolin-4-yl]amino]cyclohexyl]propyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide,
N-[1-[4-[[2-[(3RS)-2,6-Dioxo-3-piperidyl]-1,3-dioxo-isoindolin-4-yl]amino]butyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[4-[[2-[(3RS)-2,6-dioxo-3-piperidyl]-1,3-dioxo-isoindolin-4-yl]amino]butyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide,
N-[1-[4-[1-[2-[(3RS)-2,6-Dioxo-3-piperidyl]-1,3-dioxo-isoindolin-5-yl]-4-piperidyl]butyl]-4-piperidyl]-6-[[2-[(3R,4S)(3S,4R)-3-fluoro-4-methoxy-1-piperidyl]pyrimidin-4-yl]amino]-1-isopropyl-pyrrolo[3,2-c]pyridine-3-carboxamide,
N-[1-[5-[[2-[(3RS)-2,6-Dioxo-3-piperidyl]-1,3-dioxo-isoindolin-4-yl]amino]pentyl]-4-piperidyl]-6-E[2-[(3R,4 S)-3-fluoro-4-methoxy-1-piperidyl]pyrimidin-4-yl]amino]-1-isopropyl-pyrrolo[3,2-c]pyridine-3-carboxamide,
N-[1-[5-[[2-[(3RS)-2,6-dioxo-3-piperidyl]-1,3-dioxo-isoindolin-4-yl]amino]pentyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexanoyl]-4-piperidyl]-1-isopropyl-6-[[2-(4-methoxy-1-piperidyl)pyrimidin-4-yl]amino]pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexyl]-4-piperidyl]-1-isopropyl-6-[[2-(4-methoxy-1-piperidyl)pyrimidin-4-yl]amino]pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexyl]-4-piperidyl]-6-[[2-[(3R,4S)(3S,4R)-3-fluoro-4-methoxy-1-piperidyl]pyrimidin-4-yl]amino]-1-isopropyl-pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexyl]-4-piperidyl]-6-[[2-[(3 S,4R)-3-fluoro-4-methoxy-1-piperidyl]pyrimidin-4-yl]amino]-1-isopropyl-pyrrolo[3,2-c]pyridine-3-carboxamide, and
N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide.

16. The compound of claim 1, or pharmaceutically acceptable salts thereof, selected from the group consisting of:
N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrrolo[3,2-c]pyridine-3-carboxamide,
N-[1-[6-[[2-[(3RS)-2,6-dioxo-3-piperidyl]-1,3-dioxo-isoindolin-4-yl]amino]hexyl]-4-piperidyl]-6-[[2-[(3S,4R)-3-fluoro-4-methoxy-1-piperidyl]pyrimidin-4-yl]amino]-1-isopropyl-pyrrolo[3,2-c]pyridine-3-carboxamide,
N-[1-[6-[[2-[(3RS)-2,6-Dioxo-3-piperidyl]-1,3-dioxo-isoindolin-4-yl]amino]hexyl]-4-piperidyl]-6-[[2-[(3 S,4R)-3-fluoro-4-methoxy-1-piperidyl]pyrimidin-4-yl]amino]-1-isopropyl-pyrazolo[4,3-c]pyridine-3-carboxamide, and
N-[1-[6-[[2-[(3RS)-2,6-dioxo-3-piperidyl]-1,3-dioxo-isoindolin-4-yl]amino]hexyl]-4-piperidyl]-6-[[2-[(3R,4S)-3-fluoro-4-methoxy-1-piperidyl]pyrimidin-4-yl]amino]-1-isopropyl-pyrazolo[4,3-c]pyridine-3-carboxamide.

17. A pharmaceutical composition comprising a compound of claim 1 and a therapeutically inert carrier.

18. A method of treating a disorder mediated by EGFR comprising administering an effective amount of a compound of claim 1 or a pharmaceutically acceptable salt thereof to a patient in need thereof.

19. The method of claim 18, wherein the disorder mediated by EGFR is cancer.
